(19) United States
(12) Patent Application Publication
(10) Pub. No.: US 2025/0032446 A1
(43) Pub. Date: Jan. 30, 2025

(54) METHODS AND COMPOSITIONS FOR THE BIOSYNTHESIS AND USE OF DEMETHYLATED POLYMETHOXY FLAVONES

(71) Applicant: RUTGERS, THE STATE UNIVERSITY OF NEW JERSEY, New Brunswick, NJ (US)

(72) Inventors: Shiwei SU, Edison, NJ (US); Chi-Tang HO, New Brunswick, NJ (US); Qingrong HUANG, New Brunswick, NJ (US)

(73) Assignee: RUTGERS, THE STATE UNIVERSITY OF NEW JERSEY, New Brunswick, NJ (US)

(21) Appl. No.: 18/716,819

(22) PCT Filed: Dec. 12, 2022
(86) PCT No.: PCT/US2022/081349

§ 371 Date: Jun. 5, 2024

Related U.S. Application Data
(60) Provisional application No. 63/288,319, filed on Dec. 10, 2021.

(51) Int. Cl. A61K 31/353; A61K 36/07; A61P 3/06; C12P 17/06

(52) U.S. Cl. CPC A61K 31/353; A61K 36/07; A61P 3/06; C12P 17/06

(57) ABSTRACT

A novel yeast strain and methods of use thereof for robust biosynthesis of demethylated polymethoxy flavones are disclosed. Also provided are method of administering demethylated polymethoxy flavones for the treatment of obesity and inflammatory a disease.

### CROSS REFERENCE TO RELATED APPLICATIONS

This patent application claims the benefit of U.S. Provisional Patent Application No. 63/288,319, filed Dec. 10, 2021. The entire contents of the foregoing application are incorporated herein by reference, including all text, tables, drawings, and sequences.

### FIELD OF THE INVENTION

This invention relates to methods and compositions comprising the biosynthesis of demethylated polymethoxy flavones. More specifically, this invention relates to the biosynthesis of hydroxylated PMFs (OH-PMFs), particularly 6-OH and 7-OH-PMFs and use of the same for the treatment and prevention of diseases.

### BACKGROUND OF THE INVENTION

Several publications and patent documents are cited throughout the specification in order to describe the state of the art to which this invention pertains. Each of these citations is incorporated by reference herein as though set forth in full.

Polymethoxyflavone or PMF is a general term for flavones bearing two or more methoxy groups on their basic benzo-7-pyrone (15-carbon, C6-C3-C6) skeleton with a carbonyl group at the C4 position. PMFs exist almost exclusively in citrus plants. Current annual worldwide citrus production is estimated at over 105 million tons, with more than half of this being oranges. For example, total citrus production of United States was 7.78 million metric tons in 2019-2020 (National Agricultural Statistics Service). Around 34% of these products were used for juice production, yielding approximately 44% (4-5 billion lbs in US) of peels as by-products. In some regions of the world, citrus peel like orange peel or tangerine peel has been a traditional medicine for relieving stomach upset, cough, skin inflammation, muscle pain, and ringworm infections, as well as for lowering blood pressure. PMFs, rich in citrus peels, are one of the major constituents except terpenoids and other volatile oils. PMFs have been of particular interest due to their documented broad spectrum of biological activity, including anti-inflammatory (Murakami et al., 2000b, Murakami et al., 2005, Lai et al., 2007), anti-carcinogenic (Murakami et al., 2000a, Murakami et al., 2005, Lai et al., 2007, Tang et al., 2007), and anti-atherogenic properties (Kurowska and Manthey, 2004, Saito et al., 2007).

Hydroxylated PMF or OH-PMF, form a special subgroup of PMFs with one or two hydroxyl groups on the PMF skeleton together with multiple methoxy groups. Interestingly, OH-PMFs commonly show more potent activity than their PMFs counterparts in the prevention and treatment of diseases (Chiou et al., 2018; Li, & Pan et al., 2007, 2009; Guo et al., 2017; Lai et al., 2008). In the past few years, OH-PMFs were identified as demethylated metabolites of their PMF counterparts formed in a natural transformation process or in in-vivo or in-vitro metabolism pathways (Li, Lo, & Ho, 2006; Koga et al., 2011; Li, Sang et al., 2007; Okuno, & Miyazawa, 2004, 2006).

Nobiletin (NOB), (5,6,7,8,3′,4′-hexamethoxyflavone) is a dietary polymethoxylated flavonoid found in Citrus fruits. Several recent studies show that NOB and derivatives thereof act as multifunctional pharmaceutical agents. The various pharmacological activities of NOB include neuroprotection, cardiovascular protection, antimetabolic disorder, anticancer, anti-inflammation, and antioxidation. These events may be underpinned by modulation of signaling cascades, including PKA/ERK/MEK/CREB, NF-κB, MAPK, Ca²⁺/CaMKII, PI3K/Akt1/2, HIF-1α, and TGFβ signaling pathways. Other data show that certain metabolites of NOB exhibit stronger beneficial effects than NOB on disease pathogenesis.

Sinensetin, a plant-derived polymethoxylated flavonoid found in Orthosiphon aristatus var. aristatus and several citrus fruits, has been found to possess strong anticancer activities and a variety of other pharmacological benefits and promising potency in intended activities with minimal toxicity.

Tangeretin is a polymethoxylated flavone that is found in tangerine and other citrus peels. Tangeretin strengthens the cell wall and acts as a plant's defensive mechanism against disease-causing pathogens. It has also been used as a marker compound to detect contamination in citrus juices.

To date, neither chemical synthesis nor bio-synthesis procedures have been reported for the robust production of OH-PMFs comprising a hydroxyl group on C6, C7 or C8 positions of the A ring. Since OH-PMFs can be more potent than their parent PMFs in the prevention and treatment of diseases, an efficient method for synthesis of 6-OH-, 7-OH- or 8-OH-PMFs is needed. Compared with chemical synthesis, biological synthesis which is free from environmental pollution and high disposal costs is much more attractive.

It is an object of the invention to provide compositions and methods for robust generation of OH-PMFs with the hydroxyl group on the C6, C7, and C8 positions of the A ring.

### SUMMARY OF THE INVENTION

As noted above, OH-PMFs are typically more potent than their parent PMFs in the prevention and treatment of diseases. Accordingly in accordance with the present invention an efficient biological synthetic method for production of 6-OH-, 7-OH- or 8-OH-PMFs is provided.

In one aspect, the invention provides a method of producing hydroxylated polymethoxyflavone metabolites (OH-PMFs). An exemplary method comprises culturing Filobasidium magnum in the presence of a polymethoxyflavone (PMF) under conditions which permit the hydroxylation of said PMF metabolites. In certain embodiments, the Filobasidium magnum is the strain deposited as NRRL Deposit No. Y-68198. In certain aspects the metabolite is an OH-PMF listed in Table 8. In preferred embodiments, said OH-PMF is a least one of 6-OH-PMF or 7-OH-PMF. The method can further comprise extracting the OH-PMF metabolite. In preferred embodiments, the PMF is at least one of nobiletin, sinensetin, tangeretin and heptamethoxyflavone.

In certain embodiments, the PMF is dissolved in DMSO prior to contact with the yeast. The PMF can be in an aqueous solution with a stabilizer. In preferred embodiments, the PMF is in a nanodispersion. In particularly preferred embodiments, the nanodispersion is prepared using a media-milling technique.

In another aspect, methods for modulating adipolipogenesis, lipogenesis, and adipogenesis are provided comprising administering to a mammal, such as a human in need thereof, an effective amount of 6-OH-nobiletin, 7-OH-nobiletin or a combination thereof, in a pharmaceutically acceptable carrier, said administration ameliorating adipolipogenesis, lipogenesis, and adipogenesis symptoms in said mammal. In certain embodiments, the combination of 6-OH-nobiletin and 7-OH-nobiletin are administered in a ration of 1:40 and 1:50. In certain embodiments, the 6-OH-nobiletin or 7-OH-nobiletin or a combination thereof are produced by the methods disclosed above. In certain embodiments, obesity symptoms are assessed following administration of the 6-OH-nobiletin or 7-OH-nobiletin or a combination thereof.

### BRIEF DESCRIPTION OF THE DRAWINGS

FIG. 1: Chemical structure of polymethoxyflavones. Table 1 provides a listing of the moieties present on the PMFs disclosed herein.

FIG. 2A-2B: HPLC profiles of culture media after 36 hours of incubation with the fungal isolate of the invention demonstrating formation of demethylated nobiletin. (FIG. 2A) before enzymolysis, (FIG. 2B) after enzymolysis. Peaks at 21.2 min represents tangeretin as the internal standard.

FIG. 3: Phylogenetic tree of ITS region-based of the isolate with related yeast species in NCBI database using Neighbor-Joining algorithm identifying the isolate as Filobasidium magnum. The numbers (bootstrap values as percentages of 1000 replications) provide support for the robustness of the adjacent nodes.

FIG. 4: Effect of pH on the total content of extracted demethylated-nobiletins (M2; 7-OH-nobiletin, M3; 6-OH-nobiletin).

FIG. 5: Emulsion formation at different pH of fermented media after adding ethyl acetate.

FIG. 6A-6C: Silica gel chromatogram of nobiletin, M2; 7-OH-nobiletin, and M3; 6-OH-nobiletin compounds in mixtures of hexane and ethyl acetate at different ratios(v/v).5:5 as the only eluent (FIG. 6A), 6:4 (FIG. 6B) and then 5:5 (FIG. 6C); flow rate; 2 mL/min⁻¹; detection wavelength: 320 nm.

FIG. 7: The yeast survival rate with various amount of DMSO/nobiletin solution in the media. The survival ratio was expressed as the viability of yeast in the experimental group versus the control group.

FIG. 8: The nobiletin transformation efficiency with different concentrations of DMSO/nobiletin for 11 days of incubation.

FIG. 9: The yield of demethylated-nobiletin with different concentrations of DMSO/nobiletin after certain days of cultivation with the yeast.

FIG. 10: Change in particle size and polydispersity of formulated nobiletin-HMS dispersion as a function of time.

FIG. 11: The nobiletin-water solubility of nobiletin-HMS dispersion with different milling times.

FIG. 12: The yeast survival rate with various amount of HMS/nobiletin dispersion in the media. The survival ratio was expressed as the viability of yeast in the experimental group versus the control group.

FIG. 13: The nobiletin transformation efficiency with different concentrations of HMS/nobiletin for 11 days of incubation.

FIG. 14: The yield of demethylated-nobiletin with different concentrations of HMS/nobiletin after certain days of cultivation with the yeast.

FIG. 15: Bulk extraction efficiency rates by using DMSO/nobiletin solution or HMS/nobiletin dispersion in the fermentation system.

FIG. 16: Effect of nobiletin and M67N on 3T3-L1 preadipocytes proliferation. 3T3-L1 cells were incubated in complete culture medium containing with or without serial doses of the compounds for 24 hours. Data were interpreted as percent of control. Data shown reflect the means±SD of 6 experiments.

FIG. 17A-17C: Effect of nobiletin and M67N on lipid accumulation in differentiating mouse adipocytes. 3T3-L1 cells were incubated in DMEM full medium (negative control) or differentiation medium containing 0.1% DMSO (positive control) or target compounds. (FIG. 17A) photos of culture plate after Oil Red staining; (FIG. 17B) Light microscopic images of stained cells; (FIG. 17C) Stained lipid droplets were extracted with isoproponal containing 4% NP-40, and quantified by measuring the absorbance under 520 nm, which is interpreted as the relative lipid content in differentiated adipocytes. Data shown reflect the means±SD of 3 experiments. (*) P<0.05, (**) P<0.01, and (***) P<0.001. All error bars, SD.

FIG. 18A-18C: Lipid accumulation in differentiating mouse adipocytes under the treatment of M67N at early phase, late phase and full phase. 3T3-L1 cells were incubated in DMEM full medium (negative control) or differentiation medium containing 0.1% DMSO (positive control) or target compounds. (FIG. 18A) photos of culture plate after Oil Red staining; (FIG. 18B) Light microscopic images of stained cells; (FIG. 18C) Stained lipid droplets were extracted with isoproponal containing 4% NP-40, and quantified by measuring the absorbance under 520 nm, which is interpreted as the relative lipid content in differentiated adipocytes. Data shown reflect the means±SD of 3 experiments. (*) P<0.05, (**) P<0.01, and (***) P<0.001. All error bars, SD.

FIG. 19A-19F: Effect of M67N on the Mitotic clonal expansion of 3T3-L1 preadipocytes. 3T3-L1 cells were incubated in differentiation medium with or without serial doses of the compounds for 36 hours. (FIG. 19A) Data were interpreted as percent of control. (FIG. 19B) Percentages of cell population distribution. (FIG. 19C-19F) DNA histogram of cell cycle distribution of 3T3-L1 cells. Charts indicate statistical significance (p<0.05, n=3) by ANOVA.

FIG. 20: HPLC profile of 7-OH-nobiletin and 6-OH-nobiliten mixture.

FIG. 21A-21C: HPLC profile and chemical structure of nobiletin (FIG. 21A), 7-OH-nobiletin (FIG. 21B) and 6-OH-nobiliten (FIG. 21C). Note: sample 7-OH-nobiletin contains of 98% 7-OH-nobiletin and 2.0% 6-OH-nobiliten; sample 6-OH-nobiliten contains of 0.5% 7-OH-nobiletin and 99.5% 6-OH-nobiliten.

FIG. 22: Effect of nobiletin, 7-OH-nobiletin and 6-OH-nobiliten on 3T3-L1 preadipocytes proliferation. 3T3-L1 cells were incubated in complete culture medium containing with or without serial doses of the compounds for 24 hours. Data were interpreted as percent of control. Data shown reflect the means±SD of 6 experiments.

FIG. 23A-23C: Effect of nobiletin, 7-OH-nobiletin and 6-OH-nobiliten on lipid accumulation in mature adipocytes. 3T3-L1 cells were incubated in DMEM full medium (negative control) or differentiation medium containing 0.1% DMSO (positive control) or target compounds. (FIG. 23A) photos of culture plate after Oil Red staining; (FIG. 23B) Light microscopic images of stained cells; (FIG. 23C) Stained lipid droplets were extracted with iso-propanol containing 4% NP-40, and quantified by measuring the absorbance under 520 nm, which is interpreted as the relative lipid content in differentiated adipocytes. Data shown reflect the means±SD of 3 experiments. (**) P<0.01. All error bars, SD.

FIG. 24: Lipid accumulation in differentiating mouse adipocytes under the treatment of 6-OH-nobiliten at early phase, late phase and full phase.

FIG. 25A-25F: Effect of 6-OH-nobiliten on the Mitotic clonal expansion of 3T3-L1 preadipocytes. 3T3-L1 cells were incubated in differentiation medium with or without serial doses of the compounds for 36 hours. Data were interpreted as percent of control. (FIG. 25A) Effect of 6-OH-nobiliten on the viability of post-confluent 3T3-L1 cells; (FIG. 25B) Percentages of cell population distribution; (FIG. 25C-25F) DNA histogram of cell cycle distribution of 3T3-L1 cells. Charts indicate statistical significance (p<0.05, n=3) by ANOVA.

FIG. 26A-26D: Effect of 6-OH-nobiliten on the expression of early transcription factors and lipogenic genes. 3T3-L1 cells of early treatment groups were harvested at day 3 for the immunoblotting analysis of expression levels of PPARγ, C/EBPα (FIG. 26A) and p-AMPK, AMPK (FIG. 26B); 3T3-L1 cells in the late-treatment group were harvested at day 8 for the immunoblotting analysis of expression levels of ACC, FAS (FIG. 26C), and p-AMPK, AMPK (FIG. 26D). The expression level of the conservative protein 3-actin was used as a loading control.

### DETAILED DESCRIPTION OF THE INVENTION

Hydroxylated polymethoxyflavones (OH-PMFs), a subset of the polymethoxyflavone (PMF) family, exhibit a broad range of bioactivities. Because of the limited quantities available in nature, OH-PMFs are typically produced through semi-chemical or semi-biological synthesis from their PMF counterparts. However, techniques for producing OH-PMFs with the hydroxyl group on the A-ring of flavone skeleton are limited. Disclosed herein is an isolated strain of yeast, Filobasidium magnum, (NRRL Deposit No. Y-68198) which metabolizes nobiletin obtained from aged orange peel. Internal transcribed spacers (ITS) rRNA sequencing technology results confirmed that this newly isolated yeast strain belonged to specie of Filobasidium magnum.

HPLC and HPLC-MS confirmed that the metabolites produced were mono-demethylated nobiletins with the hydroxyl groups at positions of C6, C7 or C8, respectively. Two thirds of the metabolites were in the form of glycosides which were formed through the esterification reaction of one molecule aglycone and one molecule of hexose. H¹ and C¹³ NMR results indicated that the metabolites were 7-OH-nobiletin and 6-OH-nobiletin respectively. Moreover, this novel yeast strain can use multiple PMFs and OH-PMFs as substrates for mono-demethylation

We demonstrate that this strain of yeast is able to efficiently synthesize 7-OH- and 6-OH-PMFs, thereby making sufficient quantities of these agents available for investigative and therapeutic uses.

Due to the hydrophobic nature, PMFs can be dissolved in DMSO before using. However, the accessibility of the PMF for demethylation by the F. magnum can be limited because of the toxicity of DMSO as well as PMF itself to the yeast. Herein, a nobiletin nanodispersion composed of only the stabilizer-hydrophobically modified starch (HMS) - and nobiletin was developed through the media-milling technique. Results showed that the particle size of nobiletin nanodispersion was reduced to around 800 nm after 3 hours of milling process. The fabricated nanodispersion enhanced the water solubility of nobiletin by over five-fold.

We demonstrate that, compared with the DMSO/nobiletin solvent system, nobiletin nanodispersion could achieve higher production of the 6-OH and 7-OH metabolites.

### Definitions

The present subject matter may be understood more readily by reference to the following detailed description which forms part of this disclosure. It is to be understood that this invention is not limited to the specific products, methods, conditions or parameters described and/or shown herein, and that the terminology used herein for the purpose of describing particular embodiments by way of example only and is not intended to be limiting of the claimed invention.

Unless otherwise defined herein, scientific and technical terms used in connection with the present application shall have the meanings that are commonly understood by those of ordinary skill in the art. In addition to definitions included in this sub-section, further definitions of terms are interspersed throughout the text.

In this invention, “a” or “an” means “at least one” or “one or more,” etc., unless clearly indicated otherwise by context. The term “or” means “and/or” unless stated otherwise. In the case of a multiple-dependent claim, however, use of the term “or” refers back to more than one preceding claim in the alternative only.

The term “about” as used herein refers to a value that is no more than 10% above or below the value being modified by the term. For example, the term “about 5%” means a range of from 4.5% to 5.5%.

As used herein, the term “isolated” in the context of a compound or composition that can be obtained from a natural source, e.g., plants, refers to a compound or composition that is separated from one or more components from its natural source, preferably, a compound or composition that is substantially free of natural source cellular material, e.g., plant cellular material, or contaminating materials from the natural source, e.g., cell or tissue source, from which it is obtained. The language “substantially free of natural source cellular material” or substantially free of plant cellular material includes preparations of a compound that has been separated from cellular components of the cells from which it is isolated. Thus, an “isolated” compound or composition is in a form Such that its concentration or purity is greater than that in its natural source. For example, in certain embodiments, an “isolated compound or composition can be obtained by purifying or partially purifying the compound or composition from a natural Source. In some embodiments, an “isolated” compound or composition is obtained in vitro in a synthetic, biosynthetic or semisynthetic organic chemical reaction mixture.

The term “polymethoxyflavone” or “PMF” means, unless otherwise indicated, a compound having the formula:

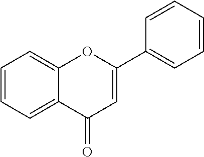 [SMILES: O=c1cc(-c2ccccc2)oc2ccccc12]

wherein at least one carbon, preferably two or more carbons, in the formula are substituted with a —OCH₃ group (in place of one or more hydrogen atoms, not depicted in the formula) as valency permits. As will be clear in the context that the term PMF is used, a PMF may be optionally substituted with Substituents, such as, for example, hydroxyl, halide, monosaccharide, or other groups, attached to one or more carbons not substituted with a methoxy group.

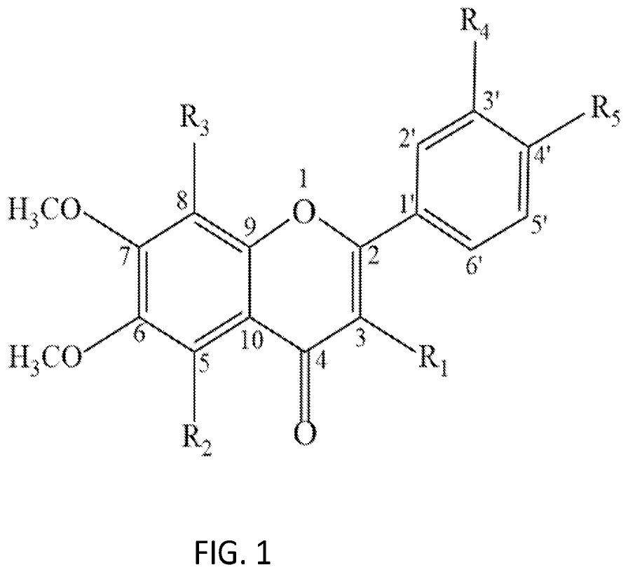

Extracted polymethoxyflavones have been studied for a wide range of potential medicinal purposes, e.g., inhibition of growth of cancer cells, anti-inflammatory activity, anti-oxidative activity, prevention of cardiovascular or cerebrovascular diseases, inhibition of pathogen growth, and other medicinal applications. PMF metabolites, such as hydroxylated polymethoxyflavones, have been shown to exhibit stronger beneficial effects than their PMF counterpart. The chemical skeleton of PMFs is shown in FIG. 1 and Table 1.

In certain embodiments, the PMF is nobiletin. Nobiletin is 5,6,7,8,3′,4′-hexamethoxyflavone and has the following chemical formula:

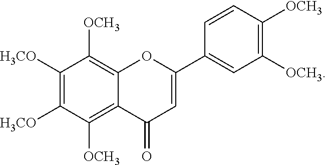 [SMILES: COc1ccc(-c2cc(=O)c3c(OC)c(OC)c(OC)c(OC)c3o2)cc1OC]

Nobiletin has been shown to have various pharmacological activities including, without limitation, neuroprotection, cardiovascular protection, antimetabolic disorder, anticancer, anti-obesity, anti-inflammation, and antioxidation.

In certain embodiments, the PMF is sinensetin. Sinensetin, 2-(3,4-dimethoxyphenyl)-5,6,7-trimethoxy-4H-1-benzopyran-4-one has the following chemical formula:

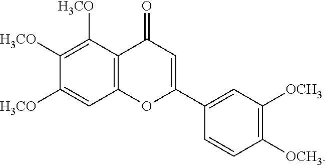 [SMILES: COc1ccc(-c2cc(=O)c3c(OC)c(OC)c(OC)cc3o2)cc1OC]

Similar to nobiletin, sinensetin exhibits various pharmacological activities, including, without limitation, anticancer, anti-inflammatory, antioxidant, antimicrobial, anti-obesity, anti-dementia, vasorelaxant and antitrypanosomal activities.

In certain embodiments, the PMF is tangeretin. Tangeretin, 5,6,7,8-tetramethoxy-2-(4-methoxyphenyl)chromen-4-one has the following chemical formula:

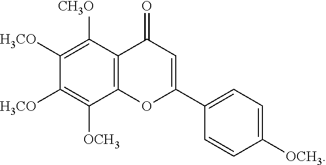

Tangeretin exhibits various pharmacological activities including, without limitation, anti-tumor activity and neuroprotective action, and hypolipidemic activity.

In certain embodiments, the PMF is “heptamethoxyflavone” or “hepta”. Hepta is chemically known as 3,5,6,7,8,3′,4′-heptamethoxyflavone and has the following chemical formula:

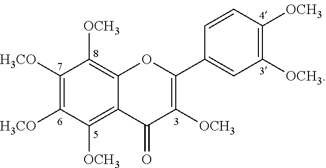 [SMILES: COc1ccc(-c2oc3c(OC)c(OC)c(OC)c(OC)c3c(=O)c2OC)cc1OC]

Hepta's various pharmacological activities include, without limitation, anti-inflammation, anti-cancer, anti-allergy, anti-aging (photoprotection), anti-depression and neuroprotection.

The term “hydroxylation” refers to a chemical process that introduces a hydroxyl group into an organic compound. The degree of hydroxylation refers to the number of hydroxyl groups in the molecule. A molecule that has undergone hydroxylation is hydroxylated. A molecule that has one hydroxyl group is mono-hydroxylated. A molecule that has more than one hydroxyl group is poly-hydroxylated.

The term “Hydroxylated polymethoxyflavones” or “OH-PMFs” refers to metabolites of PMFs that are demethylated with a hydroxyl group found at the demethylated position. In certain embodiments, the OH-PMF is mono-demethylated. In other embodiments, the OH-PMF is poly-demethylated.

As used herein, the terms “microbial,” “microbial organism” or “microorganism” refer to any organism that exists as a microscopic cell that is included within the domains of archaea, bacteria or eukarya. Therefore, the term is intended to encompass prokaryotic or eukaryotic cells or organisms having a microscopic size and includes bacteria, archaea and eubacteria of all species as well as eukaryotic microorganisms such as yeast and fungi. The term also includes cell cultures of any species that can be cultured for the production of a biochemical.

According to the current invention a “yeast” as claimed herein are eukaryotic, single-celled microorganisms classified as members of the fungus kingdom. Yeasts are unicellular organisms which evolved from multicellular ancestors but with some species useful for the current invention being those that have the ability to develop multicellular characteristics by forming strings of connected budding cells known as pseudo hyphae or false hyphae. In certain embodiments, the yeast is Filobasidium magnum. In certain embodiments, the yeast is the Filobasidium magnum with NRRL Deposit No. Y-68198.

The term “modulation” or “modulated” as used herein refers to a change, e.g., an increase or decrease, of a cell associated activity as compared to cell associated activity in the absence of the modulation methods.

The terms “non-naturally occurring” or “engineered” are used interchangeably and indicate the involvement of the hand of man. The terms, when referring to nucleic acid molecules or polypeptides mean that the nucleic acid molecule or the polypeptide is at least substantially free from at least one other component with which they are naturally associated in nature and as found in nature.

The term “media milling” refers to a popular nanodispersion-producing method that is aimed to improve the stability and bioavailability of target compounds (Bier et al., 2017). Recently, nano-dispersion system which is composed of only nano/micrometer particles (as stabilizer) and the targeted bioactive compounds has been developed in the media-milling process.

Hydrophobically modified starch (HMS) is used as an amphiphilic stabilizer that is a widely used to encapsulate hydrophobic compounds. Many results proved that this nano-dispersion system stabilized by HMS through the media-milling process demonstrated greatly improved water solubility, bio-accessibility of water-insoluble compounds (Yeh et al., 2010).

### PMF Preparation

In one aspect, provided herein are methods of preparing hydroxylated PMF-enriched plant extract compositions. The term “enriched, as used herein in connection to a “hydroxylated PMF-enriched plant extract composition, encompasses a plant extract composition wherein hydroxylated PMFs in the plant extract composition comprise at least 15% to about 95% of the total weight of the plant extract composition, and the proportion of hydroxylated PMFs to non-hydroxylated PMFs in plant extract composition is greater than the proportion hydroxylated PMFs to non-hydroxylated PMFs found naturally in the plant from which the extract is derived. In certain embodiments, a “hydroxylated PMF-enriched plant extract composition comprises at least 15%, at least about 20%, at least about 25%, at least about 30%, at least about 40%, at least about 45%, at least about 50%, at least about 55%, at least about 60%, at least about 65%, at least about 70%, at least about 75%, at least about 80%, at least about 85%, at least about 90%, or at least about 95% hydroxylated PMFs of the total weight of the composition.

Starting materials for preparing hydroxylated PMF-enriched plant extract compositions typically include an extract or isolate from a natural source that comprises PMFs. In general, sources of PMFs, such as orange peels, for instance, have a PMF fraction in which non-hydroxylated PMFs are more abundant than hydroxylated PMFs. Hence, in some embodiments, methods are provided for increasing the proportion of hydroxylated PMFs to non-hydroxylated PMFs in a plant extract.

The starting materials for the methods provided herein can be a natural source, typically a plant, plant part, or extract of a plant or plant part, such as an extract of Sap, bark, peel, rind, seed, root, juice, leaf flower, bud, etc., from a plant that naturally contains a measurable PMF component. Citrus products, for example, are a readily available source for obtaining PMFs. In certain embodiments, the plant extract is an orange peel extract, for example, an extract from cold-pressed orange peel oil solids.

In certain embodiments, the starting material is an orange peel extract having about 10% or about 20% to about 75% PMFs. The PMF fraction can, for example, consist of non-hydroxylated PMFs or comprise non-hydroxylated PMFs and hydroxylated PMFs. The starting material can, for example, be in a liquid form, such as oil or suspension, or in a dried form, such as a powder or paste, and the like.

In certain embodiments, the PMF is dispersed or dissolved in water or an organic solvent. In certain embodiments, the PMF is dissolved in an organic solvent prior to contact with the yeast. In certain embodiments, the organic solvent is nontoxic to the yeast. PMF are generally hydrophobic. Accordingly in certain embodiments, the solvent is capable of dissolving polar and nonpolar compounds. In certain embodiments, the organic solvent is dimethylsulfoxide (DMSO).

In certain embodiments, the PMF is dispersed in an aqueous solution. In certain embodiments, the solubility of the PMF is enhanced by nanodispersion, A nanodispersion refers to dispersed systems that contain nanoparticles. In certain embodiments, the nanodispersion is prepared by media-milling a stabilizer and the PMF. In certain embodiments, the stabilizer is hydrophobically modified starch (HMS). In certain embodiments, the PMF nanodispersion is reduced to less than 1000 nm In certain embodiments, the PMF nanodispersion is reduced to about 800 nm. In certain embodiments, the nanodispersion is milled for about 0-5 hours. In certain embodiments, the nanodispersion is milled for about 3 hours. In certain embodiments, the solubility of the PMF is increased at least 5-fold by the nanodispersion. In certain embodiments, the solubility of the PMF is increased by 1-fold, 2-fold, 3-fold, 4-fold, or 5-fold.

### Culture Conditions

Optimized fermentation conditions, such as batch fermentation conditions include regulation of the culture temperature and pH. For example, the culture can be maintained at a temperature wherein the substrate is efficiently used and the OH-PMF is maximally produced with minimal waste production. In certain embodiments, the agar medium comprises: Chenpi (orange peel) powder 20 g/L, glucose 10 g/L, sucrose 10 g/L, beef extract 0.2 g/L, peptone 10 g/L, yeast extract 10 g/L, MgSO₄ 0.05 g/L, KH₂PO₄ 2 g/L, NaCl 0.5 g/L. In certain embodiments, the media has a pH between about 4.5 to about 7.5. In certain embodiment the pH is about 4.5, about 6.0, or about 7.5. In certain embodiments, the culture is incubated between about 20° C. and about 37° C. In certain embodiments, the culture is incubated at about 20° C., at about 26° C. or at about 37° C. In certain embodiments, the yeast is allowed to ferment for between about 2 to about 11 days, before being isolated and purified.

### Separation of PMF

In some embodiments, the methods of preparing OH-PMF provided herein further comprise extracting the composition from the media after fermentation. Appropriate solvents for extraction are those that are non-miscible with the solvent within which the product is dissolved or dispersed. For example, in embodiments where ethanol is the solvent used to dissolver or disperse the starting material prior to heating, then solvents such as ethyl acetate can be used for extracting the product after heating. Appropriate solvents will be selected by those of skill in the art.

In certain embodiments, the fermented culture is added to a solvent mixture. In certain embodiments, the solvent mixture is hexane: ethyl acetate at a ratio of 0:3, 3:7, 4:6, 5:5, 6:4 or 7:3. In certain embodiments the fermented culture is extracted by mixing the solution. In certain embodiments, the solution is mixed for at least 2 hours. In certain embodiments, the supernatant is separated from the solution after extraction. In certain embodiments, the supernatant is separated using a separatory funnel. In certain embodiments, the extraction is performed multiple times. In certain embodiments, the extraction is performed at least 3 times. In certain embodiments, the supernatant is dried after extraction. In certain embodiments, the purity of the OH-PMF is analyzed using HPLC.

The Examples below are provided to illustrate certain embodiments of the invention. They are not intended to limit the invention in any way.

### Methods of Treatment Using OH-PMFs

Methods of inhibiting adipogenesis, in a subject in need thereof, are provided herein. In certain embodiments, the method of inhibiting adipogenesis comprises administration of an effective amount of at least one OH-PMF. In certain embodiments, the at least one OH-PMF is a 7-OH-PMF and/or a 6-OH-PMF. In certain embodiments, the at least one OH-PMF is selected from Table 1. In certain embodiments, the at least one OH-PMF is a mixture of at least one 7-OH-PMF and at least one 6-OH-PMF. In certain embodiments, the mixture is 7-OH-nobiletin and 6-OH-nobiletin. In certain embodiments, the ratio of 7-OH-PMF to 6-OH-PMF is between 1:3, 1:5, 1:10, 1:20, 1:30, 1:40, 1:50, and 1:100. In certain embodiments, the ratio of 7-OH-PMF to 6-OH-PMF is 1:49. In certain embodiments, the OH-PMF is present in a pharmaceutically acceptable carrier.

Methods of treating obesity, in a subject in need thereof, are provided herein. In certain embodiments, the method of treating obesity comprises administration of an effective amount of at least one OH-PMF. In certain embodiments, the at least one OH-PMF is a 7-OH-PMF and/or a 6-OH-PMF. In certain embodiments, the at least one OH-PMF is selected from Table 1. In certain embodiments, the at least one OH-PMF is a mixture of at least one 7-OH-PMF and at least one 6-OH-PMF. In certain embodiments, the mixture is 7-OH-nobiletin and 6-OH-nobiletin. In certain embodiments, the ratio of 7-OH-PMF to 6-OH-PMF is between 1:3, 1:5, 1:10, 1:20, 1:30, 1:40, 1:50, and 1:100. In certain embodiments, the ratio of 7-OH-PMF to 6-OH-PMF is 1:49. In certain embodiments, the OH-PMF is present in a pharmaceutically acceptable carrier.

By “treatment” and “treating” is meant the medical management of a subject with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. It is understood that treatment, while intended to cure, ameliorate, stabilize, or prevent a disease, pathological condition, or disorder, need not actually result in the cure, ameliorization, stabilization or prevention. The effects of treatment can be measured or assessed as described herein and as known in the art as is suitable for the disease, pathological condition, or disorder involved. Such measurements and assessments can be made in qualitative and/or quantitative terms. Thus, for example, characteristics or features of a disease, pathological condition, or disorder and/or symptoms of a disease, pathological condition, or disorder can be reduced to any effect or to any amount.

The term “in need of treatment” as used herein refers to a judgment made by a caregiver (e.g. physician, nurse, nurse practitioner, or individual in the case of humans; veterinarian in the case of animals, including non-human mammals) that a subject requires or will benefit from treatment. This judgment is made based on a variety of factors that are in the realm of a care giver's expertise, but that includes the knowledge that the subject is ill, or will be ill, as the result of a condition that is treatable by the disclosed compounds.

The terms “subject,” “individual,” and “patient” are used interchangeably herein, and refer to an animal, for example a human, to whom treatment, including prophylactic treatment, with the pharmaceutical composition according to the present invention, is provided. The term “subject” as used herein refers to human and non-human animals.

The term “obesity” refers to a medical condition in which excess body fat has accumulated to the extent that it may have a negative effect on health, leading to reduced life expectancy and/or increased health problems, such as increased risk of heart disease, type 2 diabetes, obstructive sleep apnea, certain types of cancer, and osteoarthritis. In certain embodiments, the obesity is treated by prevent the accumulation of excess body fat and/or decreasing the rate in which excess body fat is accumulated. In certain embodiments, the obesity is treated by decreasing the amount of excess body fat in the subject. In certain embodiments, obesity is treated by modulating adipogenesis and/or lipogenesis.

The term “lipogenesis” refers to the synthesis of fatty acids from nonlipid precursors. Lipogenesis is a pathway for metabolism of excess carbohydrate and is activated by high carbohydrate availability. In energy sufficient states, glucose is converted to pyruvate through glycolysis and pyruvate is imported into the mitochondria to join TCA cycle. Citrate formed in the TCA cycle is transported into the cytosol where it is converted to acetyl-CoA by ATP citrate lyase. De novo synthesis of fatty acids in liver begins with ATP-dependent carboxylation of acetyl-CoA to malonyl-CoA by acetyl-CoA carboxylase 1 (ACC1). Malonyl-CoA which serves as a two-carbon donor is added to the acetyl-CoA primer by a multifunctional enzyme complex, the fatty acid synthase (FAS). Thus, the fatty chain grows by the attachment of acyl residue with elongation by two carbon subunits each cycle.

The term “adipogenesis” refers to the process by which fat-laden cells develop and accumulate as adipose tissues at various sites in the body as subcutaneous fat and as fat depots. The major roles of adipocytes are to store energy as fat during periods when energy intake exceeds expenditure and to mobilize this stored fuel when energy expenditure exceeds intake. Adipocytes arise both during late embryonic development and in the mature animal under conditions that promote obesity. Obesity, the excessive accumulation of body fat, is a consequence of adipogenesis due to persistent energy intake that exceeds energy expenditure.

“Effective amount” or “therapeutically effective amount” are used interchangeably herein, and refer to an amount of a compound, formulation, material, or composition, as described herein effective to achieve a particular biological result or provides a therapeutic or prophylactic benefit. Such results may include, but are not limited to, anti-tumor activity as determined by any means suitable in the art.

The term “modulate” as used herein refers to the ability of a compound to change an activity in some measurable way as compared to an appropriate control. As a result of the presence of compounds in the assays, activities can increase or decrease as compared to controls in the absence of these compounds. Preferably, an increase in activity is at least 25%, more preferably at least 50%, most preferably at least 100% compared to the level of activity in the absence of the compound. Similarly, a decrease in activity is preferably at least 25%, more preferably at least 50%, most preferably at least 100% compared to the level of activity in the absence of the compound. A compound that increases a known activity is an “agonist”. One that decreases, or prevents, a known activity is an “antagonist”. In certain embodiments, the compounds described herein modulate lipid levels in said patient.

The phrase “pharmaceutically acceptable carrier” refers to a material that is not biologically or otherwise undesirable, i.e., the material can be administered to a subject along with the selected compound without causing any undesirable biological effects or interacting in a deleterious manner with any of the other components of the pharmaceutical composition in which it is contained. The carrier is all components present in the pharmaceutical formulation other than the active ingredient or ingredients. See, e.g., Remington's Pharmaceutical Sciences, latest edition, by E.W. Martin Mack Pub. Co., Easton, Pa., which discloses typical carriers and conventional methods of preparing pharmaceutical compositions that can be used in conjunction with the preparation of formulations of the compounds described herein and which is incorporated by reference herein. These most typically would be standard carriers for administration of compositions to humans. In one aspect, humans and non-humans, including solutions such as sterile water, saline, and buffered solutions at physiological pH. Other compounds will be administered according to standard procedures used by those skilled in the art.

### Example 1

### Biosynthesis of OH-PMFs

### Materials and Methods

Substrate pretreatment

Aged orange peel (Chenpi) from different years 2005, 2010, 2014, 2016, 2017 were purchased from Xinhui, Guangzhou, China. These peels were smashed separately using a coffee grinder and then sieved to get Chenpi powders between 0.5 to 1.0 millimeters. The Chenpi powders were packaged in polyethylene bags and kept in desiccator until use.

### Isolation of Microorganisms

Chenpi of each year were cut into pieces and imprinted on agar plates. The agar medium was composed of the following composition (g/L): Chenpi powder 20, glucose 10, sucrose 10, beef extract 0.2, peptone 10, yeast extract 10, MgSO₄ 0.05, KH₂PO₄ 2, NaCl 0.5. The media was adjusted to pH 4.5, 6.0, or 7.5 before autoclaving. After incubation at 20° C., 26° C. or 37° C. for 2 to 11 days, representative colonies of each morphological type were isolated and purified in the same agar medium.

Selection of hydroxyl-nobiletin producers was performed in flasks containing the isolated microorganisms, nobiletin (final concentration of 54 μg/mL in the media), and the following medium (g/L): glucose 20, polypeptone 10, yeast extract 5, NaCl 0.5, MgSO₄ 0.05, and KH₂PO₄ 2. After incubation at 26° C. for 3 days, media was taken out for HPLC analysis.

### Strain Identification

ITS sequences were used to identify the isolates, where primers ITS1(Forward ITS 5′-TCC GTA GGT GAA CCT GCG G-3′ (SEQ ID NO:1)) and ITS4 (5′-TCC TCC GCT TAT TGA TAT GC-3′ (SEQ ID NO:2)) were used to amplify this region by PCR (Yerou et al., 2017). The obtained sequences were aligned and compared with those of reference yeast strains in BLAST search. The phylogenetic tree was constructed based on the Neighbor-Joining method excluding positions with gaps (Wikandari et al., 2015). Maximum Composite Likelihood was employed for the evolutionary analyses by using software MEGA7.

### Analysis of Metabolic Compounds

### Cultivation of Microorganisms

Colonies of the isolate from slants were transferred into sterile culture media and kept on a gyratory shaker (with temperature set at 26° C.) for 10 hours to give stage I culture. 1 mL of stage I culture was added to another flask with 50 mL sterile culture media. After 6 to 8 hours incubation at 26° C., stage II culture was obtained which had OD 600 nm falling between 0.5 and 0.8; subsequently, substrate nobiletin was added and incubated in 23° C.

### Fermentation of Nobiletin

150 μL nobiletin dissolved in DMSO (18 mg/mL) was added into stage II culture (250 mL flask with 50 mL media). Incubation was conducted at 180 rpm orbital shaking for 11 days. 5 mL fermentation cultures were taken out for enzymic hydrolysis.

### Enzymic Hydrolysis

Fermentation cultures were adjusted to pH 5.0 using hydrochloric acid. 0.2 M acetate buffer at pH 5.0 was added to obtain the final concentration of 0.02 M. The media was then incubated with P-glucosidase from Sweet Almonds (MP Biomedicals) at 37° C. Complete hydrolysis was observed after 2 hours of incubation. The mixture was subjected for extraction and HPLC analysis.

### Extraction of Nobiletin Metabolites

0.5 mL fermentation sample and 0.7 mL ethyl acetate were added to a 1.5 mL centrifugation tube and vortexed for 10 minutes. The tube was then centrifuged at 5000 rpm for 3 minutes. 0.55 mL supernatant was then removed into a clean tube. This process was repeated two additional times, and then the supernatant was collected for evaporation. After evaporation, 0.2 mL methanol was added to re-dissolve the dried residuals. After being filtered (0.22 m Nylon filter), HPLC was applied to measure the concentration of nobiletin metabolites.

### HPLC Analysis

The extracted samples were analyzed by the Agilent 1100 HPLC system using a Phenomenex Synergi 4 Hydro-RP column (250×4.6 m) connected to a variable wavelength detector. A linear gradient elution program composed of water (solvent A) and acetonitrile (solvent B) was carried out as follows: 0 min, 25% solvent B; 3 min, 25% solvent B; 28 min, 100% solvent B; 30 min, 25% solvent B. The flow rate was kept at 1 mL/min and injection volume was 20 μL. UV detection wavelengths were set at 320 nm.

### Large Scale Fermentation for the Production of Nobiletin Metabolites

To obtain the purified demethylated nobiletins for the chemical structural analysis, a large-scale fermentation was carried out. 100 mL nobiletin dissolved in DMSO (18 mg/mL) was evenly divided into 40 flasks (500 mL) that contained 250 mL stage II cultures. After 11 days of fermentation at 23° C., the incubation mixture was adjusted to pH 2.0 using HCl and autoclaved for 20 minutes to de-glucosidate glucosidic demethylated-nobiletins. The extractant (mixtures of hexane and ethyl acetate) was then extracted multiple times until the final concentration of nobiletin in the incubation mixture was lower than 0.5 μg/mL. The pooled organic phase was evaporated until dry, dissolved with ethyl acetate, and separated using silica gel chromatography.

### Extraction Optimization: Hexane to Ethyl Acetate Ratios as Extractant, Extractant to Sample Ratios, and pH of Sample

First, 500 mL fermented culture was mixed with extractant (hexane:ethyl acetate=0:3, 3:7, 4:6, 5:5,6:4 and 7:3, v/v) for extraction. The flask with culture and extractant were stirred on a plate using a magnetic stirrer for 2 hours at room temperature. To evaporate the solvent during extraction, the samples were covered with 3 layers of aluminum foil and the extraction process was carried out in a fume hood. After extraction, the supernatant was separated with a separatory funnel. The extraction was carried out 3 times. Supernatants were combined, dried by rotatory evaporation and weighted. The residual was redissolved with 25 mL methanol and analyzed using HPLC after the filtration with a 0.45 m filter. The purity was defined as the ratio of total amount of target compounds (nobiletin and metabolites) to the weight of the dried residual. The total amount of target compounds which was extracted by using 100% ethyl acetate as extractant was set as the standard (100% extraction efficiency). The amount of target compounds that were obtained by using other extractant mixtures divided by the standard was regarded as the extraction efficiency of the responding extractant.

After the determination of optimum extractant mixtures. An experiment was carried out on the volume ratios of extractant to sample (1:1, 1:2, 1:5, 1:8 or 1:10). Specifically, 500 mL fermented culture was added with 500 mL, 250 mL, 100 mL, 62.5 mL and 50 mL extracts. Multiple extractions were performed until the final concentration of nobiletin in the culture was lower than 0.5 μg/mL. The consumption of total solvent in volume, and the number of extractions were recorded for each sample.

Lastly, the fermented media was adjusted to pH 2.0, 5.0, 6.5, 7.0, 8.0 and 10.0 using HCl or NaOH. Subsequently, 3 extractions were conducted, and the sample was dried, weighed and analyzed using HPLC. The amount of extracted metabolites were compared and used to evaluate the effect of pH on extraction.

### Silica Gel Chromatography

Extracted samples (5 g) were dissolved in 15-30 mL ethyl acetate and loaded onto a 125 g preconditioned silica gel column (9 cm×180 cm, I.D. silica gel 100-200 mesh). Isocratic elution was then applied with a mixture of hexane and ethyl acetate (6:4 or 5:5, v/v) at a constant flow of 2 mL/min. Eluent was collected every 50 mL in one tube and tested by thin layer chromatography (TLC). TLC analyses were conducted on silica gel plates at room temperature, using hexane: ethyl acetate (3:7, v/v) as developing reagent. Spots were visualized with an ultraviolet lamp at 280 nm. Fractions that contained target compounds were analyzed using HPLC for quantification. Meanwhile, factions were combined into two groups, one with nobiletin and another one with the metabolites. After being concentrated under reduced pressure, the dried residues were further separated using reversed phase preparative HPLC.

### Preparative HPLC

The residue of demethylated nobiletins was dissolved in methanol and loaded to a preparative HPLC (Waters 152) which was equipped with a 2489 detector and an YMC ODS-A column (250 mm×20 mm, 5 m particle size). An isocratic elution of 30% water and 70% 30 methanol was used in 25 min with a flow rate of 20 mL/min. The pure fractions combined and concentrated or lyophilized to dryness. The dried compounds were analyzed by NMR and LC-MS.

### LC-ESI-MS

LC/MS experiments were performed on a system consisting of ion trap mass spectrometer with an ESI ionization interface and an Agilent 1100 HPLC system. ESI experiments were carried out in the positive mode; Mass range measured, m/zl50-m/z1000; ion trap temperature 250° C.; EM 3.5 kV; drying N² 10 mL/min; nebulizing N² 30 psi.

Analytical HPLC conditions on HPLC-MS was carried out using a Phenomenex Synergi 4 u Hydro-RP column (250×4.6 mm) and an eluent, (A) H₂O, (B) acetonitrile. The gradient elution started with B being 40% which increased linearly and ended with B reaching 78% within 15 min. The injection volume was 1 μL with the flow rate being 0.3 mL/min and UV detection wavelengths set at 320 nm.

### NMR Characterization

NMR spectra were recorded on a Bruker AVANCE NEO 500 with tetramethylsilane as the internal standard. ¹H NMR was recorded 500 MHz while ¹³C NMR was recorded at 126 MHz.

### Substrate-Selectivity of CYP450s

Apart from nobiletin, multiple compounds were fermented in stage II culture media. These compounds included three additional PMFs —sinensetin, tangeretin, heptamethoxyflavone (hepta), and 10 demethylated-PMFs including 3′-demethyl-nobiletin, 4′-demethyl-nobiletin, 3′4′-di-demethyl-nobiletin, 3′5′-di-demethyl-nobiletin, 4′5′-di-demethyl-nobiletin, 5-demethyl-nobiletin, 4′-demethyl-tangeretin, 5-demethyl-tangeretin, 4′-demethyl-hepta, 5-demethyl-heptamethoxyflavone. Each of these compounds were dissolved in DMSO, and the final concentration in the media was 24 μg/mL.

Chemical structures of the substrates are in FIG. 1 combined with Table 1.

TABLE 1
Chemical structure of tested PMFs

| Compound | R1 | R2 | R3 | R4 | R5 |
|---|---|---|---|---|---|
| Sinensetin | H | OMe | H | OMe | OMe |
| 3′-demethyl-nobiletin | H | OMe | OMe | H | OMe |
| 4′-demethyl-nobiletin | H | OMe | OMe | OMe | H |
| 5-demethyl-nobiletin | H | H | OMe | OMe | OMe |
| 3′4′-di-demethyl-nobiletin | H | OMe | OMe | H | H |
| 3′5-di-demethyl-nobiletin | H | H | OMe | H | OMe |
| 4′5-di-demethyl-nobiletin | H | H | OMe | OMe | H |
| Nobiletin | H | OMe | OMe | OMe | OMe |
| 4′-demethyl-heptamethoxyflavone | OMe | OMe | OMe | H | OMe |
| 5-demethyl-heptamethoxyflavone | OMe | OMe | OMe | OMe | H |
| Heptamethoxyflavone | OMe | OMe | OMe | OMe | OMe |
| 4′-demethyl-tangeretin | H | OMe | OMe | H | OMe |
| 5-demethyl-tangeretin | H | H | OMe | H | OMe |
| Tangeretin | H | OMe | OMe | H | OMe |

*me = methyl

After 4 days of cultivation, fermented cultures with and without enzyme hydrolysis were analyzed with HPLC and HPLC-MS. Demethylation rate and glycosidation rate were respectively calculated using the following equations.

$$Demethylation Rate = \frac{Initial substrate concentration - Final substrate concentration}{Initial substrate concentration} \times 100 \%$$

$$and$$

$$Glycosidation Rate = \frac{Aglycon concentration after enzymolysis - Aglycon concentration before enzymolysis}{Aglycon concentration after enzymolysis} \times 100 \%$$

### Statistical Analysis

Experimental data were presented as mean±standard error. The significant difference among groups was tested using student test or one-way ANOVA followed by Tukey test using SPASS and reported as different letters if p-value<0.05.

### Results

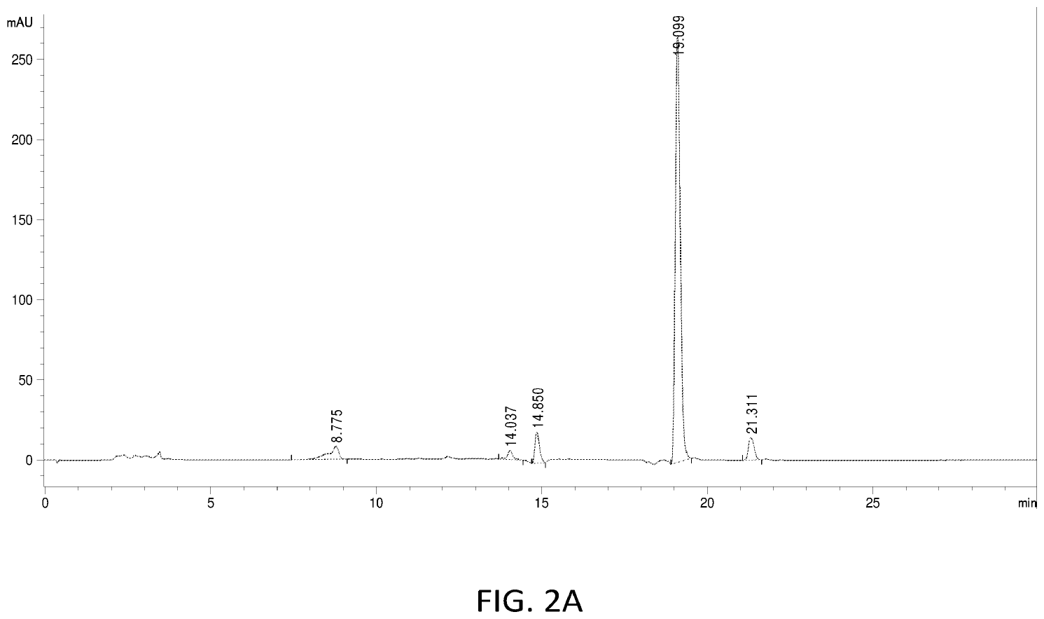

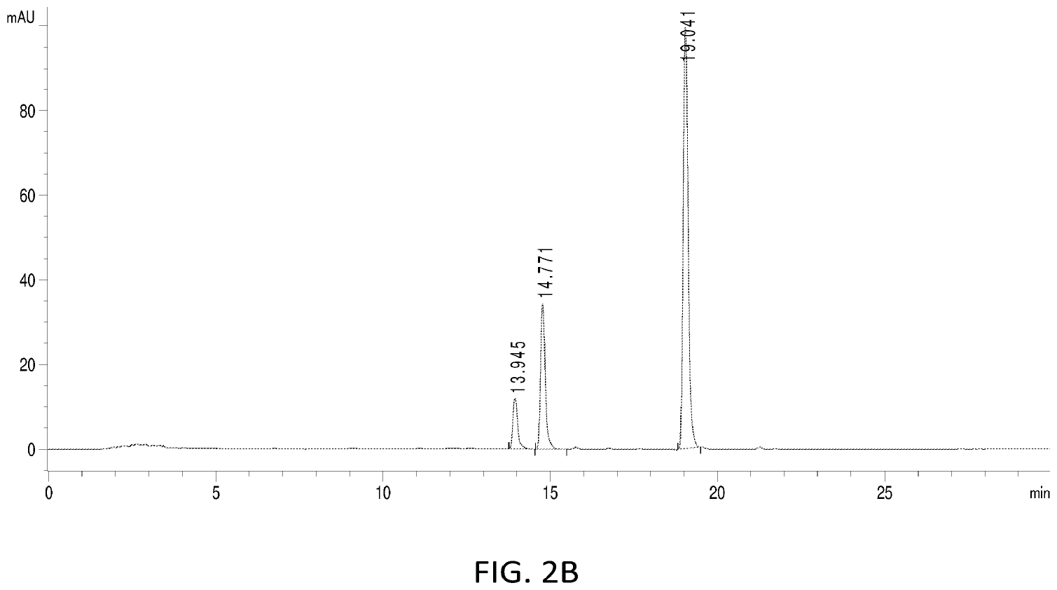

Isolation and Characterization of Yeast strain producing demethylated nobiletin For the screening of microorganisms that had the ability to demethylate nobiletin on the A ring, all isolated and purified colonies were separately picked and inoculated in the liquid media that contained substrate nobiletin. After 2 to 5 days of incubation, cultures were sampled and analyzed by HPLC. Results as shown in FIGS. 2A and 2B demonstrated that the HPLC profile of one culture media changed: three peaks which stood for three new compounds (M1, M2 and M3) and respectively had retention time at 8.7 min, 14.0 min and 14.8 min appeared before nobiletin (RT 19.2 min). Meanwhile, the quantitative analysis using standard curve demonstrates that the concentration of nobiletin in the media decreased from 158 μg/mL to 60 μg/mL. These results indicate that the three compounds are the metabolites of nobiletin under the effect of the isolate.

Identification of the Isolate that Demethylated Nobiletin

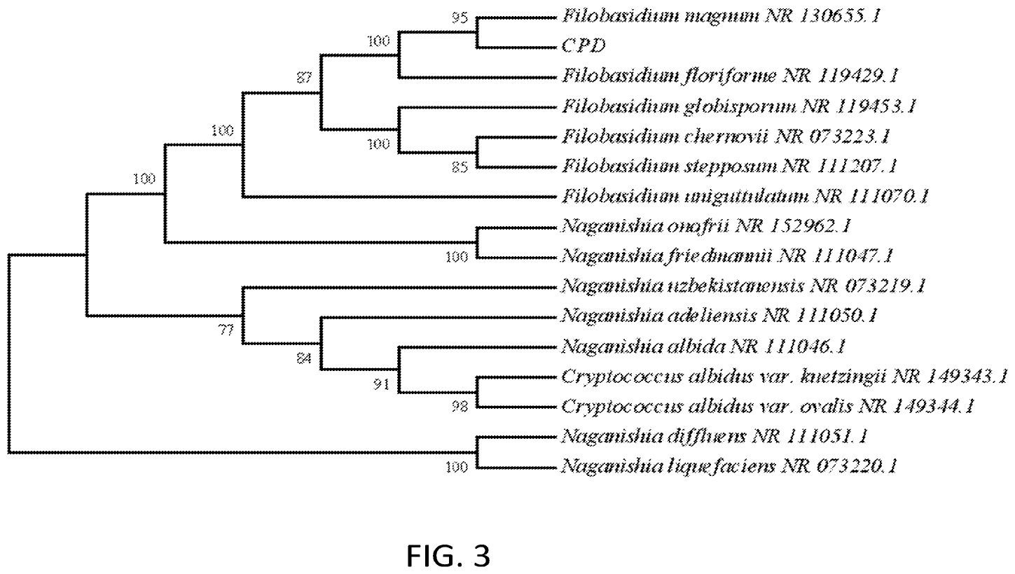

The new strain of microorganism that was able to metabolize nobiletin was isolated. For the identification of this isolate, the isolate's ITS sequences were obtained through RCR and then clustered for similarity with reference ITS sequences from GenBank (BLAST search). As shown in the phylogenetic analysis in FIG. 3, the isolate (labeled as CPD) clustered with identified microorganisms Filobasidium magnum (F. M) with the bootstrap value 95. This FM has been deposited as NRRL Deposit No. Y-68198.

### HPLC and HPLC-MS Analysis of the Metabolites of Nobiletin

For the characterization of nobiletin metabolites, HPLC-MS was performed to determine the molecular weight. Results showed that both M2 (7-OH-nobiletin), and M3 (6-OH-nobiletin) have a molecular ion [M+H]⁺ at 389 Da which was 14 Da less than that of nobiletin (403 Da).

This indicates that mono-demethylation occurred at two different positions of the benzene skeleton, releasing two distinct mono-demethylated nobiletins. M1 was characterized with molecular ion [M+H]⁺ of 551, and fragment ion of 389. 551 is a sum of 388+162+H where 162 is a typical fragment of hexose after losing one molecular of H₂O. This indicates that M1 is a mono-demethylated nobiletin glycoside formed through the esterification reaction of one molecule aglycone and one molecule of hexose.

It has been previously reported that the produced demethylated-PMFs in vivo or in vitro would suffer glycosidation and be transformed into their glycosides. Therefore, P-glucosidase was used to release the aglycone. As shown in FIG. 2B, the peak at 8.5 min disappeared after enzymic hydrolysis while the absorbance of M2 and M3 separately increased from 2.3 μg/mL to 3.5 μg/mL and from 6.9 μg/mL to 18.8 μg/mL. This indicates that M1 was the glucoside of M2 and M3. Notably, the concentration ratio of the two compounds M2: M3 was approximately 1:3 (6.9 μg/mL:18.8 μg/mL). This phenomenon indicates that for the regioselectivity of this demethylation enzyme from the isolated yeast: M2 was a preferable metabolite. To uncover the demethylation positions, available OH-nobiletin standards: 4′-OH-, 3′-OH-, 5-OH-, and 3′,4′-diOH-nobiletin were used as HPLC standards. The retention time and molecular weight was presented in Table 2. The four compounds showed retention time separately at 15.8 min, 15.8 min, 22.4 min and 12.9 min. None of them was consistent with the retention time of M2 or M3. These results indicate that the demethylation occurred at two of the three positions C6, C7 or C8 on the A ring.

TABLE 2
Retention time and molecular weight comparison
of the two nobiletin metabolites with standards

| Compounds | Retention time(min) | Molecular Weight(Da) |
|---|---|---|
| M1 | 8.7 | 550 |
| 3′4′-OH-Nobiletin | 12.9 | 374 |
| M2 | 13.6 | 388 |
| M3 | 14.8 | 388 |
| 3′-OH-Nobiletin | 15.8 | 388 |
| 4′-OH-Nobiletin | 15.8 | 388 |
| Nobiletin | 19.2 | 402 |
| Tangeretin | 21.5 | 432 |
| 5-OH-Nobiletin | 22.4 | 388 |

### Optimization of Extraction Process

### Optimization of Hexane to Ethyl Acetate Ratio as Extractant

Generally, culture product obtained by microbial conversion has a strong unpleasant odor, dark color and low purity (Hama et al. US patent, 2012). The issue is worsened when the extractant used was pure ethyl acetate even though it is one of the most common and efficient extractants. This is due to the high hydrophilic nature of ethyl acetate, which leads to the incorporation of several unwanted compounds such as phospholipid, and polysaccharides in the extracts (Hamada et al., US patent, 2014). Notably, the solvent hexane is much more hydrophobic but less efficient in extracting PMFs. It is also commonly used for extraction with the purpose of decreasing the content of unwanted compounds in the extracts. Therefore, to suppress the incorporation of impurities with no loss of target compound concentration, a comparative study on the purity of extracts and the extraction efficiency was carried out by applying multiple extractants that had a hexan to ethyl acetate ratio (H:EA) of 1:10, 3:7, 4:6, 5:5, 6:4 and 7:3. The results were shown in Table 3.

TABLE 3
Purity of target compounds (nobiletin and metabolites)
and the extract efficiency of different extractants.*

| H:EA | 0:10 | 3:7 | 4:6 | 5:5 | 6:4 | 7:3 |
|---|---|---|---|---|---|---|
| Purity | 35.3 ± 1.8% | 53.0 ± 1.2% | 55.8 ± 0.7% | 64.7 ± 0.6% | 70.4 ± 2.1% | 72.7 ± 1.1% |
| Extract | 100% | 100% | 100% | 100% | 100% | 94 ± 1.5% |
| efficiency | | | | | | |

*Extractant to sample ration 1:1; 3 times extraction

As shown in Table 3, 35.3±1.8% purity was obtained when using pure ethyl acetate as the extractant. As percent of hexane in the extractant increased, the purity of extracts was improved and reached 70.4±2.1% as the ratio of hexane to ethyl acetate reached 6:4. Interestingly, the extract efficiency of those extracts (hexane to ethyl acetate ratio from 3:7 to 6:4) were the same with that of pure ethyl acetate. The mixture with hexane to ethyl acetate being 7:3 gave higher purity (72.7±1.1%), but the exact efficiency suffered an abrupt decrease to 94±1.5%. As such, the mixture with ratio of hexane to ethyl acetate being 6:4 was selected as the final extractant for the extraction.

### Optimization of Solvent to Sample Ratio

Generally, the extraction yield could be enhanced by a large solvent volume which dissolve constituents more effectively (Li, Chen & Yao, 2005). However, this will cause a huge waste of organic solvent and increase the labor in the concentration process. However, the yield of target compounds would be tremendously reduced if small amounts of solvent were used (Valachovic et al., 2001). With the purpose of maximizing the extraction efficiency while lowering the consumption of solvents, experiments were carried out on the solvent to sample (500 mL) ratio and total solvent consumed.

TABLE 4
Total solvent consumption based on different
volume rations of solvent to sample*

| Solvent:sample(v:v) | 1:1 | 1:2 | 1:5 | 1:7.5 | 1:10 |
|---|---|---|---|---|---|
| Extraction times | 3 | 3 | 4 | 6 | >8 |
| Solvent consumption(mL) | 1500 | 750 | 400 | 400 | >400 |

*sample volume 500 mL

Table 4 shows that when the solvent to sample ratio was set at 1:1, only three extractions were needed. However, 1500 mL of solvent was consumed. When the ratio was 1:2, the solvent consumption decreased to 750 mL. Interestingly, if the ratio was decreased to 1:5, 1:8 or 1:10, 400 ml or 350 mL of solvent was consumed with four, five or seven extractions. Considering the extraction time and solvent consumption, 1:5 (solvent to sample, v/v) was selected as the optimum ratio. This method calls for 4 extractions and 400 mL of total solvent for each 500 ml sample media.

### Optimal pH of Fermented Culture for Extraction

pH plays a critical role on the extraction of flavonoids. On one hand, acidic conditions encourage the interaction of flavonoids with polysaccharides and nucleophilic groups on proteins present in the extract (Liang, 2001) and therefore reduce the extraction efficiency. On the other hand, alkaline conditions usually lead to the degradation or oxidation of flavonoids (Chethan & Malleshi, 2007; Kim, 1999).

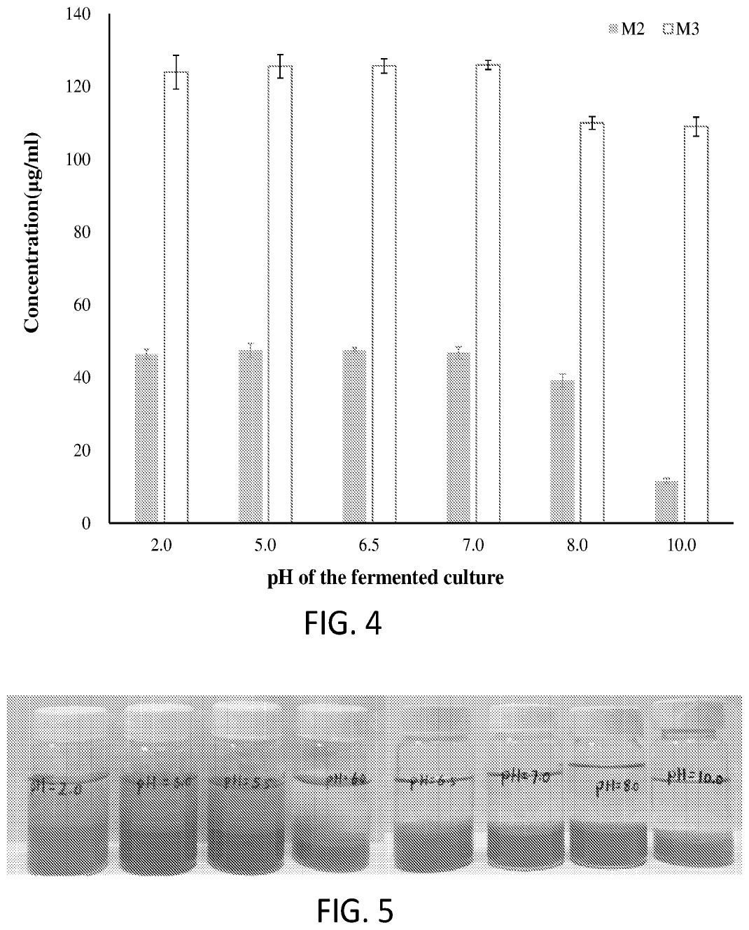

As shown in FIG. 4, within the pH range 2.0 to 7.0, no significant difference was shown on the total amount of extracted M2 and M3 with values separately being 46.5±1.3 μg/mL, 47.5±1.9 g/mL, 47.6±0.7 μg/mL, 46.9±1.5 μg/mL, and 123.9±4.6 μg/mL, 125.6±3.2 μg/mL, 125.8±1.9 g/mL, 125.9±1.2 μg/mL. Therefore, pH (from 2.0 to 7.0) had no apparent influence on the extraction of M2 and M3. However, as pH was increased to 8.0, the content of M2 decreased to 39.2±1.8 μg/mL and that of M3 fell to 110±1.7 μg/mL. With pH being 10.0, the content of M2 decreased abruptly to 11.7±0.6 μg/mL, while that of M3 (109±2.6 μg/mL) showed no significant difference with that at pH 8.0.

The results revealed that pH within the range 2.0 to 7.0 exerted no influence on the extraction of M2 and M3. The results were inconsistent with other reported results that indicated that flavonoids tend to bond with proteins and polysaccharides at low pH and thereby affecting the extraction (Chethan & Malleshi, 2007; Kim, 1999). This might be attributed to the specific interaction mechanisms of different flavonoids, proteins, and other large molecules. For example, different flavonoids might connect with large molecules through different interactions, like ionic bonds, hydrogen bonds or hydrophobic interactions. And the interaction could be changed by the pH or salt concentration (Su et al., 2016; 2018).

Flavonoids, especially those with hydrogen groups, are easily oxidized at alkaline conditions (Chethan & Malleshi, 2007; Su et al., 2018). That is why the content of M2 and M3 suffered decrease at pH 8.0. At pH 10.0, the content of M2 decreased further. M2 and M3 were not stable at alkaline condition, which might be due to the hydrogen group on the skeleton. Furthermore, M3 was more susceptible to high pH, like 10.0.

Emulsion formation usually occurs during the extraction because of the existence of proteins or lysed cells in the media (Manderfeld et al., 1997), which decreases the extraction efficiency. The phenomenon is more prominent in acid conditions. Thus, the emulsion formation was compared when the extraction was conducted with different pH of the media. As shown in FIG. 5, very poor emulsion formation was observed when the pH media was lower than 5.5. In contrast, when the pH was 6.0 or higher, a clear layer between the water and organic solvent phase was shown. Therefore, with the purpose of eliminating the degradation of metabolites and suppress the emulsion formation, the media was adjusted to pH 6.0-7.0 before extraction.

### Optimization of Silica Gel Chromatography Separation

As shown in Table 3, the extracts from fermented batch culture contained high levels of impurities. Additionally, the dried residuals have a dark color and strong odor. To improve the purity of target compounds and separate M2 and M3 from nobiletin, silica gel chromatography was used. The column was eluted with isocratic hexane-ethyl acetate. Chromatographic parameters including flow rate, and mobile phase composition were investigated to produce optimum separation.

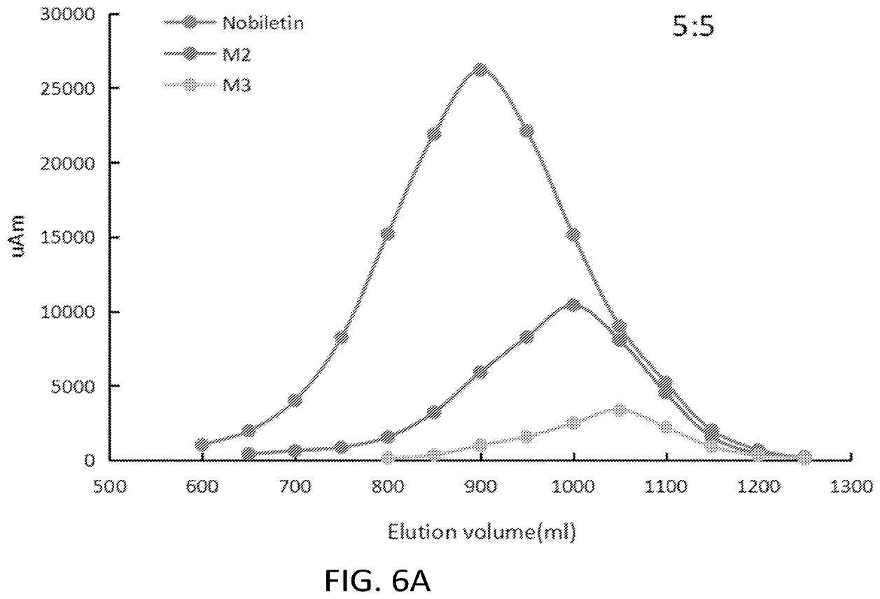

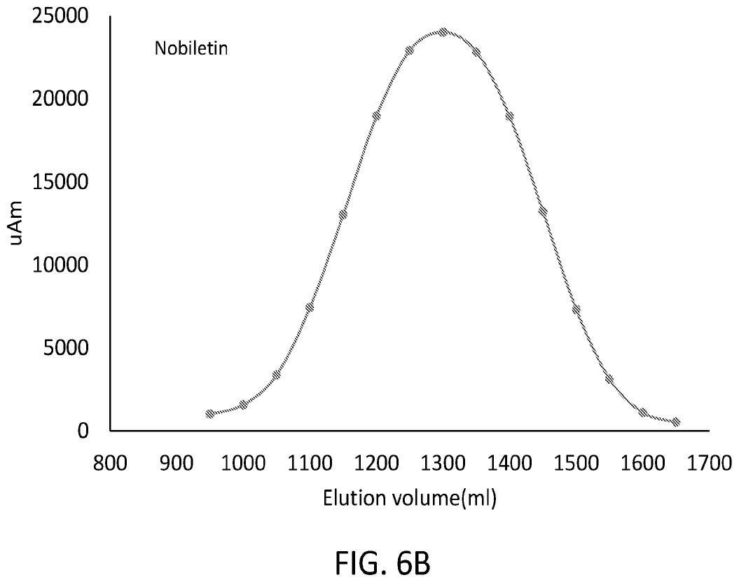

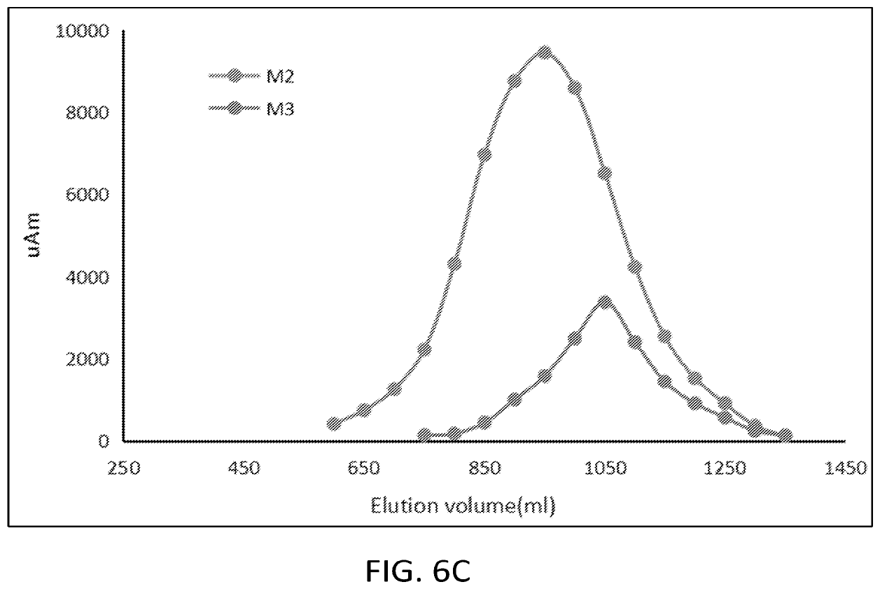

Chromatographic parameters including flow rate and mobile phase composition were investigated to produce optimum separation conditions. Results showed the ideal separation was obtained by using gradient elution. The volume ratio of hexane to ethyl acetate was 6:4 first, followed by 5:5. As shown in FIG. 6, nobiletin was firstly eluted out by hexane-ethyl acetate (6:4). The total volume used was 1200 mL. Subsequently, M2 and M3 were obtained by 850 mL of hexane-ethyl acetate (5:5) mixtures.

Mobile phase flow rate plays a critical rule on the separation efficiency (Melwita, Tsigie, Ismadji, & Ju, 2011; Gunawan, Ismadji, & Ju,2008). As flow rates was less than 2.5 mL/min, the separation efficiency and elution time gradually increased with the decreasing flow rate. This was attributed to the enhancement of diffusion capacity (Sun et al., 2014). With the flow rate larger than 3.0 mL/min, the diffuse efficiency was reduced, and impurities were brought to the collected fraction. Therefore, an ideal flow rate was selected at 2.5 mL/min. After the silica gel chromatography, the purity of M2 and M3 could separately reach 95.2% and 96.2%.

¹H and ¹³C NMR Spectra of the Metabolites

After preliminary purification using silica gel chromatography, the mixture of M2 and M3 was applied to preparative-HPLC for separation and further purification. The pure compounds M2 and M3 were analyzed by NMR for the elucidation of their chemical structures. The hydrogen and carbon resonance were presented in Table 5 (¹H NMR) and Table 6 (¹³C NMR) which evidenced the flavonoid carbon skeleton.

Five methoxy groups were characterized both on ¹H NMR (4.04 ppm, 3H; 4.00 ppm, 3H; 3.94 ppm, 3H; 3.93 ppm, 6H for 21; 4.14 ppm, 3H; 4.03 ppm, 3 H; 3.99 ppm, 3H; 3.97 ppm, 3H, 3.96 ppm, 3H for M3) and on the ¹³C NMR spectra (62.11, 61.72, 61.54, 55.99, 55.89 for M2; 62.72, 62.03, 61.44, 56.1, 55.99 for M3). The ABX pattern of ortho aromatic protons at 7.53 ppm, 6.96 ppm, 7.38 ppm (M2) or 7.55 ppm, 6.99 ppm, 7.40 ppm (M3), and they are the B-ring protons (H_{2′,5′,6′}) indicated the existence of 3′,4′-disubstituted flavones. The presence of a resonance at 6.6 ppm indicated that the C3 was not substituted (Chen, & Montanari,1998). The absence of the downfield proton resonance around 12.5 ppm, which diagnostically signal the proton at C5, evidenced that the demethylation portion was not on C5. Based on these analytical data, it can be determined that M2 and M3 had identical chemical structure of the B and C ring to their substrate nobiletin. This data confirms that the substitution position did not occur on the B ring or the C5 on the A ring.

However, as shown in Table 5, the two compounds had identical ¹H resonance of the hydroxyl group at 7.26 ppm. That is to say the ¹H spectra could not help identifying the specific structure of M2 or M3, which directs us to the ¹³C spectra as shown in Table 6. ¹³C spectra of nobiletin from chloroform was used for reference (Li, Zhao, & Zhou, 2018).

TABLE 5
¹H NMR of nobiletin metabolites (M2, M3)

| | —OH | Ar—H | | —OCH2—H |
|---|---|---|---|---|
| M2 | 6.03 (s, 1H) | 7.53 (d, J = 8.5 Hz, 1H) | C′2—H | 4.04(s, 3H) |
| | 7.26(s, 1H) | 7.38 (s, 1H) | C′5—H | 4.00(s, 3H) |
| | | 6.96 (d, J = 8.0 Hz, 1H) | C′3—H | 3.94(s, 3H) |
| | | 6.61 (s, 1H) | C3—H | 3.93(s, 3H) |
| | | | | 3.93(s, 3H) |
| M3 | 6.03 (s, 1H) | 7.55 (dd, J = 2.0, 9.0 Hz, 1H) | C′2—H | 4.14(s, 3H) |
| | 7.26(s, 1H) | 7.40 (s, 1H) | C′5—H | 4.03(s, 3H) |
| | | 6.99 (d, J = 8.5 Hz, 1H) | C′3—H | 3.99(s, 3H) |
| | | 6.63 (s, 1H), | C3—H | 3.97(s, 3H) |
| | | | | 3.96(s, 3H) |

In terms of polyoxygenated flavones, the signals at 2-, 3- and 4-positions in the C-ring were hardly influenced by the substituents on the A-ring except for those at the 5-position. As shown in Table 5, M2 and M3 shared the same basic carbon signals with nobiletin at the three positions, which further confirmed the demethylation did not happen at C5. Meanwhile, the substituents in the A-ring barely exert any effect on carbon resonance in the B-ring. Therefore, the three compounds enjoyed the same signals at C′1 to C′6 in the B-ring, as proved by Table 5. In contrast, the conversion of methoxy groups on the A-ring to hydroxy groups could lead to diagnostic shift of the A-ring carbon signals.

The demethylation at C6 would cause a significant up field shift of the ortho carbon C5 and C7 as well as the para position C9. However, its effect on the meta carbon position C8 was negligible or slightly downfield δ=+0.1 ppm. The conversion at C7 would also cause the upshift of the signal at ortho positions of C6 and C8, but barely have effect on the resonance of C5, C9 or C10.

As to the demethylation of C8, the carbon signal on its ortho position C7 and C9 as well as the para position of C5 would be greatly shifted upfield. However, it would leave the signal of C6 slightly shifted downfield δ=+0.4 ppm. The signal of the demethylated carbon position (C6 or C7 or C8) would suffer an upfield shift (Horie et al., 1998).

In the spectra comparison of M2 with nobiletin, there was an upfield shift of −0.88 ppm, −5.51 ppm, −4.14 ppm, −6.14 ppm and −0.07 ppm for C5, C6,C7, C8 and C9. Specifically, there were great upfield shifts for C6, C7 and C8 but slight change on the resonance of C5 and C9. Therefore, our data indicates that M2 was 7-OH-nobiletin. As to M3, there were upfield shifts of −8.14 ppm, −6.39 ppm, −4.1 ppm, +0.01 ppm and −2.14 ppm at C5, C6, C7, C8 and C9. Since C5, C6, C7 and C9 showed significant upfield shift while C8 had slight downfield shift, our data indicates that M3 was 6-OH-nobiletin.

TABLE 6
The real and prospect ¹³C NMR results of metabolites (M2 and M3)

| | C2 | C3 | C4 | C9 | C10 | C5 | C6 | C7 | C8 | C′1 | C'2 | C′3 | C′4 | C′5 | C′6 |
|---|---|---|---|---|---|---|---|---|---|---|---|---|---|---|---|
| M2 | 160.89 | 106.62 | 177.46 | 147.64 | 111.73 | 147.53 | 138.61 | 147.32 | 131.86 | 123.86 | 108.45 | 149.17 | 151.81 | 111.13 | 119.53 |
| M3 | 161.35 | 106.63 | 177.23 | 145.57 | 114.2 | 140.27 | 140.02 | 145.07 | 138.01 | 124.08 | 108.57 | 149.3 | 151.94 | 111.24 | 119.65 |
| Nob | 161.13 | 106.81 | 177.34 | 147.71 | 114.78 | 148.41 | 144.12 | 151.46 | 138 | 123.99 | 108.63 | 149.32 | 151.99 | 111.27 | 119.67 |

TABLE 7
¹³C NMR of nobiletin and its metabolites (M2 and M3)

OMe

| | C8 | C7 | C5 | C′4 | C′3 | |
|---|---|---|---|---|---|---|
| M1 | 62.11 | 61.72 | 61.54 | 55.99 | 55.89 | |
| | C8 | C5 | C6 | C′4 | C′3 | |
| M2 | 62.72 | 62.03 | 61.44 | 56.1 | 55.99 | |
| | C8 | C5 | C6 | C7 | C′4 | C′3 |
| Nob | 62.26 | 61.96 | 61.81 | 61.66 | 56.09 | 55.98 |

The two compounds were previously reported to be the nobiletin metabolites produced by the liver in mammals such as human beings, rats, guinea pigs and hamsters. They were also reported as metabolites of Blautia sp. MRG-PMF1 which was found in human gut (Koga, 2007&2011; Li & Sang, 2007; Burapan et al., 2017). However, this is the first report of the bio-synthesis of 6-hydroxy-5,7,8,3′,4′-pentamethoxyflavone (6-OH-nobiletin) and 7-hydroxy-5,6,8,3′,4′-pentamethoxyflavone (7-OH-nobiletin) by the yeast strain Filobasidium magnum (NRRL Deposit No. Y-68198) which catalyzed the biotransformation process with nobiletin being the substrate.

### Substrate Specialty

TABLE 8
Characterizations of metabolites of different substrates by using HPLC and ESI-MS.

| | Peaks of | t^{R}/ | | | |
|---|---|---|---|---|---|
| OH-PMFs | metabolites | min | [M + H]+ | Formula | Assignment |
| Sinensetin | 1 | 8.2 | 521.1 | C25H28O12 | mono-demethyl-sinensetin-O-glucoside |
| | 2 | 13.2 | 359.1 | C19H18O7 | mono-demethyl-sinensetin |
| | 3 | 13.6 | 359.1 | C19H18O7 | mono-demethyl-sinensetin |
| Nobiletin | 1 | 8.2 | 551.1 | C25H30O13 | mono-demethyl-nobiletin-O-glucoside |
| | 2 | 11.7 | 389.1 | C20H20O8 | mono-demethyl-nobiletin |
| | 3 | 12.8 | 389.1 | C20H20O8 | mono-demethyl-nobiletin |
| Heptamethoxyflavone | 1 | 8.5 | 581.1 | C26H32O14 | mono-demethyl-heptamethoxyflavone- |
| | | | | | O-glucoside |
| | 2 | 14.6 | 419.1 | C21H22O9 | mono-demethyl-heptamethoxyflavone |
| | 3 | 15.3 | 419.1 | C21H22O9 | mono-demethyl-heptamethoxyflavone |
| Tangeretin | 1 | 8.4 | 521.1 | C24H28O12 | mono-demethyl-tangeretin-O-glucoside |
| | 2 | 15.9 | 359.1 | C19H18O7 | mono-demethyl-tangeretin |
| | 3 | 16.9 | 359.1 | C19H18O7 | mono-demethyl-tangeretin |
| 3′-demethyl-nobiletin | 1 | 8.2 | 551.1 | C26H30O13 | 3′-demethyl-nobiletin-O-glucoside |
| 4′-demethyl-nobiletin | 1 | 8.3 | 551.1 | C26H30O13 | 4′-demethyl-nobiletin-O-glucoside |
| 3′4′-di-demethyl- | 1 | 8.03 | 537.1 | C25H28O13 | 3′4′-di-demethyl-nobiletin-O-glucoside |
| nobiletin | | | | | |
| 3′5′-di-demethyl- | 1 | 8.03 | 537.1 | C25H28O13 | 3′5′-di-demethyl-nobiletin-O-glucoside |
| nobiletin | | | | | |
| 4′5′-di-demethyl- | 1 | 8.03 | 537.1 | C25H28O13 | 4′5′-di-demethyl-nobiletin-O-glucoside |
| nobiletin | | | | | |
| 5-demethyl-nobiletin | 1 | 10.9 | 537.1 | C26H30O13 | 5,7-di-demethyl-nobiletin-O-glucoside |
| | 2 | 17.2 | 375.1 | C19H18O8 | di-demethyl-nobiletin |
| 4′-demethyl- | 1 | 8.2 | 521.1 | C25H28O12 | 4′-OH-tangeretin-O-glucoside |
| tangeretin | 2 | 11.7 | 345.1 | C18H16O7 | di-demethyl--tangeretin |
| | 3 | 12.8 | 345.1 | C18H16O7 | di-demethyl--tangeretin |
| 5-demethyl-tangeretin | 1 | 11.0 | 507.1 | C25H28O13 | di-demethyl--tangeretin-O-glucoside |
| | 2 | 19.2 | 345.1 | C18H16O7 | di-demethyl--tangeretin |
| 4′5′-di-demethyl- | 1 | 10.23 | 507.1 | C24H26O12 | 4′5′-di-demethyl-tageretin-O-glucoside |
| tangeretin | | | | | |
| 4′-demethyl- | 1 | 8.7 | 551.1 | C26H30O13 | 4′-demethyl-nobiletin-O-glucoside |
| heptamethoxyflavone | | | | | |

P450s has a fascinating ability to adapt itself to substrates of various sizes and shapes while retaining the overall P450-fold, P450 electron transfer, and P450 O₂ activation chemistries. Because of this characterization, P450s commonly has a broad range of substrates (De Montellano,2005). For example, P. chrysosporium P450s was able to metabolize various subclasses of flavonoids such as isoflavonoids, flavones, catechins, and anthocyanins (Kasai et al., 2009). Disclosed herein are 3 more kinds of PMFs including sinensetin, tangeretin, heptamethoxyflavones, and 10 types of demethyl-PMFs, which were fermentated to test the substrate specialty of Filobasidium magnum P450s.

As shown in Table 8, sinensetin, nobiletin, tangeretin and heptamethoxyflavone were respectively metabolized into 3 metabolites. LC-MS result showed that both the 2nd and 3rd metabolites featured molecular weights which were 14 Da less than their corresponding substrates, which means that one methoxy group on the different positions of the skeleton was substituted with the hydroxyl group. For example, sinensetin, which showed molecular ion [M+H]+ at 373 Da, had two metabolites (the 2nd and 3rd one) that with a molecular ion of 359 Da. Specifically, they were two different mono-demethyl-sinensetins. The first metabolite of each of the four substrates has a molecular weight ion which is 162 Da higher than that of the 2nd and 3rd metabolites. The ion with 162 Da is a typical fragment of hexose after losing one molecular H₂O. The first metabolites of each of the four PMFs might be glycosides of their 2nd and 3rd metabolites. This was proven by using P-glucosidase to release the aglycone. HPLC results (not shown) showed that metabolites M1 disappeared while the production of M2 and M3 increased. In conclusion, the four PMFs—sinensetin, nobiletin, tangeretin and heptamethoxyflavone were respectively demethylized into 2 different mono-demethyl-aglycons and these aglycons were then transformed to glycosides.

To uncover the demethylation positions, chemically synthesized OH-tangeretins and OH-heptamethoxyflavones which included 4-OH-, 5-OH-tangeretin, 3′-OH-, 4′-OH- and 5-OH-heptamethoxyflavone were used as HPLC standards. However, none of these compounds had the same retention time with the corresponding metabolite. Therefore, the data shows that demethylation occurred on the 6th or 7th or 8th position of A ring of tangeretin and heptamethoxyflavone. Referring to the demethylation position of nobiletin, the hydroxyl groups might be also on C6 or C7 of tangeretin and heptamethoxyflavone. Since sinensetin is absent from the C8 methoxy group, its two metabolites might also be 6-OH-sinensetin and 7-OH-sinensetin. In summary, the data indicates that the four major PMFs (sinesetin, nobiletin, tageretin and heptamethoxyflavone) in citrus peel are all mono-demethylated by Filobasidium magnum at the C6 and C7 of the A ring.

To test the substrate preference of Filobasidum magnum P450s among sinensetin, nobiletin, heptamethoxyflavone and tangeretin, the demethylation rates after 11 days of incubation were compared as shown in Table 9. It can be seen that tangeretin and heptamethoxyflavone could be almost totally demethylated, while only 38.5±1.4% of sinensetin was metabolized within the same days of fermentation. The main difference of sinensetin is the lack of methoxy group on C8. That is to say, the substitutional group on C8 of the skeleton might be an important factor that affect the demethylation of this compound.

Enzymes can control the regioselectivity of reactions of organic molecules. Regioselectivity refers to the preference of a functional group to bond to one atom over another atom in the reactant (Mu et al., 2020). To uncover the preferable demethylation position, the production of M2 and M3 of each of the four PMFs were compared. As demonstrated in Table 9, the ratios of the two metabolites were distinguished for each of the four substrates: production ratio of M2 to M3 were respectively 2:1, 2:3, 1:3 and 9:10 for sinesetin, nobiletin, heptamethoxyflavone and tangeretin. However, generally M3 is a favored metabolite over M2 in terms of the demethylation of nobiletin, heptamethoxyflavone and tangerentin. In contrast, M2 is more prevalent than M3 when sinesetin was used as the substrate. Notably, the regioselectivity of the Filobasidium magnum CYP 450 is also affected by chemical structures of substrates. Sinesetin lacks a methoxy group on C8 compared with the other three PMFs, this difference might be a cause for the change in enzyme regioselectivity.

Hydroxylated-PMFs with the hydroxyl group on C5 or on the B ring were applied to media of Filobasidium magnum. After 4 days of cultivation, media were sampled for HPLC analysis. As shown in Table 7, 3′-demethyl-nobiletin, 4′-demethyl-nobiletin, 3′4′-di-demethyl-nobiletin, 3′5′-di-demethyl-nobiletin and 4′5′-di-demethyl-nobiletin, 4′-demethyl-heptamethoxyflavone, were all metabolized into one metabolite which had retention time at 8.0-8.3 min. However, LC-MS results showed that the molecular ion of these metabolites were [M+162+H]+ where M denotes the molecular weight of the corresponding substrate. After the enzymolysis of beta-glucosidase, all these metabolites disappeared, and the substrates regained the initial concentrations. Thus, the 6 kinds of OH-nobiletins were not demethylated by Filobasidium magnum P450s. Instead, they were transformed into their glucosides as shown in Table 8. 4′-OH-tangeretin, 5-OH-nobiletin, and 5-OH-tangeretin reacted differently from 3′-OH-nobiletin. 46.0% of 4′-OH-tangeretin was glycosylated to its glucosides while 51.7% of it was subjected to demethylation (Table 8). For 5-OH-nobiletin, and 5-OH-tangeretin, two metabolites M1 and M2 were produced. LC-MS results (Table 8) proved M2 has a molecular weight which was 14 Da less than that of their corresponding substrate, while the molecular weight of M1 equals the sum of molecular weight of M2 plus 162 Da. In conclusion, M2 was metabolite of the substrate after losing one methyl group while M1 was the glucosides of M2.

In terms of 5-OH-PMFs, a hydrogen bond between the hydrogen atom of 5-hydroxyl group and the oxygen atom of carbonyl group is formed, producing a stable six-membered ring (Li, Pan et al., 2009). The formation of the extra ring might limit the availability of the 5-hydroxyl group for glycosylation, and therefore 5-OH-PMFs glycosides were not detected in LC-MS, as shown in Table 8. Moreover, 5-OH-PMFs underwent demethylation only on one single position, instead of two different positions. It might be the existence of the hydroxyl group on C5 inhibited the demethylation on C6 or C7. The demethylation rate of the two 5-OH-PMFs were approximately 92% which was lower that of their corresponding PMF counterparts. In conclusion, the 5-hydroxyl group can not only affect the variety of metabolites but also the production yield.

TABLE 9
The Glycosylation or demethylation rates of different substrates

| | Ratio of | Demethylation | Demethylation |
|---|---|---|---|
| PMFs | metabolites | rate (%) | rate (%) |
| Sinesetin | | — | 38.5 ± 1.4% |
| | | — | |
| Heptamethoxyflavone | 2:3 | — | 99.7 ± 0.1% |
| | | — | |
| Nobiletin | 1:3 | — | 99.2 ± 0.2% |
| | | — | |
| Tangeretin | 9:10 | — | 93.8 ± 0.7% |
| | | — | |
| 3′-demethyl-nobiletin | — | 99.8 ± 0.2% | 0 |
| 4′-demethyl-nobiletin | — | 99.7 ± 0.2% | 0 |
| 3′4′-di-demethyl-nobiletin | — | 100% | 0 |
| 3′5′-di-demethyl-nobiletin | — | 99.9 ± 0.1% | 0 |
| 4′5′-di-demethyl-nobiletin | — | 100% | 0 |
| 5-demethyl-nobiletin | — | 0 | 92.1 ± 1.2% |
| 4′-demethyl-tangeretin | 2:3 | 46.0% | 51.7% |
| 5-demethyl-tangeretin | — | 0 | 90.7 ± 2.7% |
| 4′5′-di-demethyl- | — | 99.9 ± 0.1% | 0 |
| tangeretin | | | |
| 4′-demethyl- | — | 99.8 ± 0.2% | 0 |
| heptamethoxyflavone | | | |

F. magnum metabolizes four major PFMs —sinesetin, nobiletin, tageretin and heptamethoxyflavone into their 6-demthyl and 7 demethyl metabolites. In certain cases the compounds were in the form of glycosides. The hydroxyl group on OH-PMFs had an important effect on the demethylation process. Specifically, hydroxyl groups on B ring would totally inhibit the demethylation, while OH-PMFs with C5 hydroxyl group could be only demethylated on one position. In summary, this application makes a breakthrough for the robust bio-synthesis of 6-OH- and 7-OH-PMFs, for use in new therapeutic applications and formulations.

### REFERENCES

Buisson, D., Quintin, J., & Lewin, G. (2007). Biotransformation of polymethoxylated flavonoids: access to their 4′-O-demethylated metabolites. Journal of natural products, 70(6), 1035-1038.
Chen, J., Antonio M. Montanari., Isolation and identification of new polymethoxyflavonoids from dancy tangerine leaves. Journal of Agricultural and Food Chemistry 1998. 46(4): p. 1235-1238.
Chethan, S. and N. Malleshi, Finger millet polyphenols: Optimization of extraction and the effect of pH on their stability. Food Chemistry, 2007. 105(2): p. 862-870.
Chiou, Y.-S., Yu-Nu Zheng, Mei-Ling Tsai, Ching-Shu Lai, Chi-Tang Ho, Min-Hsiung Pan, 5-Demethylnobiletin more potently inhibits colon cancer cell growth than nobiletin in vitro and in vivo. Journal of Food Bioactives, 2018. 2: p. 91-97.
De Montellano, P. R. O. (Ed.). (2005). Cytochrome P450: structure, mechanism, and biochemistry. Springer Science & Business Media.

Gunawan, S., S. Ismadji, and Y.-H. Ju, Design and operation of a modified silica gel column chromatography. Journal of the Chinese Institute of Chemical Engineers, 2008. 39(6): p. 625-633.
Guengerich, F. P. (2018). Mechanisms of cytochrome P450-catalyzed oxidations. ACS catalysis, 8(12), 10964-10976.
Guo, J., Cao, Y., Ho, C. T., Jin, S., & Huang, Q. (2017). Aged citrus peel (chenpi) extract reduces lipogenesis in differentiating 3T3-L1 adipocytes. Journal of Functional Foods, 34, 297-303.
Hama, M., Konishi, H., Watanabe, T., Onozuka, K., Koyama, S., U.S. Pat. No. 8,252,565. Washington, DC: U.S. Patent and Trademark Office, 2012.
Hamada, S., Koyama, S., Onozuka, K., Watanabe, T., U.S. Pat. No. 8,822,186. Washington, DC: U.S. Patent and Trademark Office, 2014.
Horie, T., Ohtsuru, Y., Shibata, K., Yamashita, K., Tsukayama, M., & Kawamura, Y. (1998). 13C NMR spectral assignment of the A-ring of polyoxygenated flavones. Phytochemistry, 47(5), 865-874.
Kasai, N., Ikushiro, S. I., Hirosue, S., Arisawa, A., Ichinose, H., Wariishi, H., . . . & Sakaki, T. (2009). Enzymatic properties of cytochrome P450 catalyzing 3′-hydroxylation of naringenin from the white-rot fungus Phanerochaete chrysosporium. Biochemical and biophysical research communications, 387(1), 103-108.
Kim, S.-H., et al., Effect of pH on the green tea extraction. 1999. 31(4): p. 1024-1028.
Koga, N., Ohta, C., Kato, Y., Haraguchi, K., Endo, T., Ogawa, K., . . . & Yano, M. (2011). In vitro metabolism of nobiletin, a polymethoxy-flavonoid, by human liver microsomes and cytochrome P450. Xenobiotica, 41(11), 927-933.
Lai, C. S., Li, S., Chai, C. Y., Lo, C. Y., Dushenkov, S., Ho, C. T., . . . & Wang, Y. J. (2008). Anti-inflammatory and antitumor promotional effects of a novel urinary metabolite, 3′, 4′-didemethylnobiletin, derived from nobiletin. Carcinogenesis, 29(12), 2415-2424.
Lai, C. S., et al., Disease chemopreventive effects and molecular mechanisms of hydroxylated polymethoxyflavones. Biofactors, 2015. 41(5): p. 301-13.
Li, H., B. Chen, and S. Yao, Application of ultrasonic technique for extracting chlorogenic acid from Eucommia ulmodies Oliv. (E. ulmodies). Ultrason Sonochem, 2005. 12(4): p. 295-300.
Li, S., C.Y. Lo, and C.T. Ho, Hydroxylated polymethoxyflavones and methylated flavonoids in sweet orange (Citrus sinensis) peel. J Agric Food Chem, 2006. 54(12): p. 4176-85.
Li, S., Lo, C. Y., & Ho, C. T. (2006). Hydroxylated polymethoxyflavones and methylated flavonoids in sweet orange (Citrus sinensis) peel. Journal of agricultural and food chemistry, 54(12), 4176-4185.
Li, S., Pan, M. H., Lai, C. S., Lo, C. Y., Dushenkov, S., & Ho, C. T. (2007). Isolation and syntheses of polymethoxyflavones and hydroxylated polymethoxyflavones as inhibitors of HL-60 cell lines. Bioorganic & medicinal chemistry, 15(10), 3381-3389.
Li, S., Pan, M. H., Lo, C. Y., Tan, D., Wang, Y., Shahidi, F., & Ho, C. T. (2009). Chemistry and health effects of polymethoxyflavones and hydroxylated polymethoxyflavones. Journal of Functional Foods, 1(1), 2-12.
Li, S., Sang, S., Pan, M. H., Lai, C. S., Lo, C. Y., Yang, C. S., & Ho, C. T. (2007). Anti-inflammatory property of the urinary metabolites of nobiletin in mouse. Bioorganic & medicinal chemistry letters, 17(18), 5177-5181.
Liang, Y., Xu, Y., Effect of pH on cream particle formation and solids extraction yield of black tea. Food Chemistry, 2001. 72(2): p. 155-160.
Li, Z., Zhao, Z., & Zhou, Z. (2018). Simultaneous separation and purification of five polymethoxylated flavones from “dahongpao” tangerine (Citrus tangerina tanaka) using macroporous adsorptive resins combined with prep-HPLC. Molecules, 23(10), 2660.
Manderfeld, M. M., et al., Isolation and identification of antimicrobial furocoumarins from parsley. J Food Prot, 1997. 60(1): p. 72-7.
Melwita, E., et al., Purification of Azadirachtin Via Silica Gel Column Chromatography. Journal of Liquid Chromatography & Related Technologies, 2011. 34(19): p. 2462-2472.
Mu, R., Wang, Z., Wamsley, M. C., Duke, C. N., Lii, P. H., Epley, S. E., . . . & Roberts, P. J. (2020). Application of Enzymes in Regioselective and Stereoselective Organic Reactions. Catalysts, 10(8), 832.
Okuno, Y., & Miyazawa, M. (2004). Biotransformation of Nobiletin by Aspergillus n iger and the Antimutagenic Activity of a Metabolite, 4′-Hydroxy-5, 6, 7, 8, 3′-pentamethoxyflavone. Journal of natural products, 67(11), 1876-1878.
Okuno, Y., & Miyazawa, M. (2006). Microbial O-demethylation of sinesetin and antimutagenic activity of the metabolite. Journal of Chemical Technology & Biotechnology: International Research in Process, Environmental & Clean Technology, 81(1), 29-33.
Su, S., et al., Effect of peptide-phenolic interaction on the antioxidant capacity of walnut protein hydrolysates. International Journal of Food Science & Technology, 2018. 53(2): p. 508-515.
Su, S., et al., Walnut phenolic compounds: Binding with proteins and antioxidant activities. 2016. 32(22): p. 309-314.
Sun, Y., et al., Isolation of two new prenylated flavonoids from Sinopodophyllum emodi fruit by silica gel column and high-speed counter-current chromatography. J Chromatogr B Analyt Technol Biomed Life Sci, 2014. 969: p. 190-8.
Tung, Y. C., et al., 5-Demethylnobiletin and 5-Acetoxy-6,7,8,3′,4′-pentamethoxyflavone Suppress Lipid Accumulation by Activating the LKB1-AMPK Pathway in 3T3-L1 Preadipocytes and High Fat Diet-Fed C57BL/6 Mice. J Agric Food Chem, 2016. 64(16): p. 3196-205.
Valachovic, P., A. Pechova, and T.J. Mason, Towards the industrial production of medicinal tincture by ultrasound assisted extraction. Ultrason Sonochem, 2001. 8(2): p. 111-7.
Wikandari, R., et al., Improvement of biogas production from orange peel waste by leaching of limonene. Biomed Res Int, 2015. 2015: p. 494182.
Yerou, K.O., Ibri, K., Bouhadi, D., Hariri, A., Meddah, B., Touil, A. T., The use of orange (Citrus sinensis) peel as antimicrobial and anti-oxidant agents. Journal of Fundamental and Applied Sciences, 2017. 9(3): p. 1351-1357.
Zhang, H., Tian, G., Zhao, C., Han, Y., DiMarco-Crook, C., Lu, C., . . . & Zheng, J. (2019). Characterization of polymethoxyflavone demethylation during drying processes of citrus peels. Food & function, 10(9), 5707-5717.
Zheng, G. D., et al., Rapid resolution liquid chromatography-electrospray ionisation tandem mass spectrometry method for identification of chemical constituents in Citri Reticulatae Pericarpium. Food Chem, 2013. 136(2): p. 604-11.

### Example 2

### Improved Biotransformation of PMF by Nanodispersion

### Materials and Methods

### Cultivation of Microorganisms

Colonies of the F. magnum isolate from slants were transferred into sterile culture media and kept on a gyratory shaker (with temperature set at 26° C.) for 10 h to give stage I cultures. 1 mL of stage I culture was added to another flask with 50 mL sterile culture media. After 6-8 h incubation at 26° C., stage II cultures were obtained. Stage II cultures had a OD value at 630 nm falling in absorbance between 0.5-0.8, substrate nobiletin was added.

### Analysis of Metabolic Compounds.

### Fermentation of Nobiletin

150 μL nobiletin dissolved in DMSO (18 mg/mL) was added into stage II cultures (250 mL flask with 50 mL media). Incubation was conducted at 180 rpm orbital shaking for 11 days. 500 μL fermentation cultures was taken out for enzymolysis.

### Enzyme Hydrolysis

Media were collected after the fermentation and adjusted to pH 5.0 using hydrochloric acid. 0.2 M acetate buffer was added to obtain the final concentration of 0.02 M. The media was then incubated with P-glucosidase from Sweet Almonds (MP Biomedicals) at 37° C. Complete hydrolysis was observed after 2 hours of incubation. The mixture was extraction and HPLC analysis.

### Extraction of Demethlyted Nobiletins

Get 0.5 mL fermentation samples in the 1.5 mL centrifugation tube; Add 0.7 mL ethyl acetate, and vortex for 10 mins; Centrifuge at 5000 rpm in 3 mins, and then remove 0.55 mL supernatant in a clean tube; do the extraction for another 2 time, and then collect the supernatant with the previous one for evaporation; After evaporation, add 0.2 mL methanol to re-dissolve; After being filtered (0.22 μm Nylon filter), HPLC was applied to measure the concentration of demethylated nobiletins.

### HPLC Analysis

The extracted samples were analyzed by the Agilent 1100 HPLC system using a phenomenex Synergi 4 u Hydro-RP column (250×4.6 m) connected to a variable wavelength detector. A linear gradient elution program composed of water (solvent A) and acetonitrile (solvent B) was carried out as follows: 0 min, 25% B; 3 min, 25% B; 28 min, 100% B; 30 min, 25% B. The flow rate was kept at 1 mL/min and injection volume was 20 mL. UV detection wavelengths were set at 320 nm.

### Preparation of Formulated Nobiletin Dispersions

Nobiletin dispersions were prepared by dispersing 8 g of nobiletin powder and 120 g of Hydrophobically modified starch (HMS) which acted as the media. The power was mixed in 300 mL deionized water. The suspension was under agitation for 4 hours at room temperature. HMS with the brand name Hi-Cap 100 was kindly donated by National Starch and Chemical Company, Bridgewater, NJ, USA.

### Media Milling Process

The media-milling machine (MiniCer, NETZSCH-Feinmahltechnik GmbH, Staufen, Germany) was used in this study to reduce the crystallite sizes of nobiletin. The grounding work is performed by yttrium-stabilized zirconium oxide grinding beads with a diameter of 0.8-1.0 mm acting as the milling media. The nobiletin-HMS suspension was fed to a milling chamber and ground under a pump speed of 200 rpm, mill speed of 3000 rpm and mill power of 0.13 kw. During the process, a cooling system was employed to control the milling temperature at 4° C. Samples were collected every 1 hour for particle size analysis.

### Freeze Drying Process

The milled nobiletin-HMS dispersions were applied to freeze dryer (Freezone 4.5, Labconco, Kansas City, MO, USA) for frozen drying. After all moisture was removed a dry solid mass remained (Pralhad & Rajendrakumar, 2004). Dried powders were stored in sterile centrifuge tubes at −20° C. until further use.

### Particle Size Measurements

The particle size of the nobiletin-HMS dispersions with milling time being 0 hour, 1 hour, 2 hours, 3 hours, 4 hours and 5 hours were measured by dynamic light scattering based Particle Size Analyzer (Model 90 Plus, Brookhaven Instrument Corp., Holtsville, NY, USA). The scattering angle was fixed at 90 and temperature at 25.0° C. Samples were diluted by 1000 times before the analysis. All samples were measured in triplicate to ensure accuracy.

Cultivation of the Yeast with the Presence of Various Amounts of DMSO/Nobiletin Solution or HMS/Nobiletin Dispersion in the Media

Various volumes of DMSO/nobiletin solution (18 mg/mL) were added into stage II cultures to achieve final DMSO concentrations of 0%, 0.3%, 0.6%, 1.0%, 2.0%, 4.0% and 6.0%.

10 g of freezing dried nobiletin-HMS powders were dissolved in sterile DI water to reach the final concentration of 500 mg/mL. After 2 hours of agitation (600 r/min) in room temperature, various volumes of nobiletin-HMS solution were added into stage II cultures to achieve final formula concentration of 0, 2 mg/mL, 6 mg/mL, 10 mg/mL, 15 mg/mL and 20 mg/mL.

The corresponding nobiletin concentrations are listed in the following table.

TABLE 10
Nobiletin concentrations corresponding to different concentrations
of DMSO/nobiletin and HMS/nobiletin in the media.

| | | | | | | |
|---|---|---|---|---|---|---|
| DMSO/nobiletin (%) | 0.3 | 0.6 | 1.0 | 2.0 | 4.0 | 6.0 |
| Nobiletin concentration (μg/mL) | 64 | 108 | 180 | 360 | 720 | 1080 |
| HMS/nobiletin (mg/mL) | 2 | 6 | 10 | 15 | 20 | |
| Nobiletin concentration (μg/mL) | 125 | 375 | 725 | 937.5 | 1450 | |

### The Detection of Demethylation Rate, Yeast Survival Rate and Metabolite Yield

Cultures were sampled after 0 day, 3 days, 5 days, 7 days, 9 days, 11 days of incubation for the detection of the yeast survival rate and for the HPLC analysis of demethylated-nobiletin production.

### The Demethylation Rate

The demethylation rate in this work was expressed as the nobiletin concentration ratio change in the media before and after certain days of cultivation. The equation is shown below:

$$Demethylation rate ( \% ) = 1 - \frac{nobiletin concentration in the media of different {days}^{′} cultivation}{nobiletin concentration in day 0}$$

### Metabolite Yield

The concentration of demethylated nobiletins in media was employed to indicate the metabolite yield.

### Yeast Survival Rate

Cells in each culture samples were centrifuged at 4000 g for 5 minutes and washed with the sterile PBS buffer medium (0.01 M, pH=6.8) two times. After re-suspending yeast cells in PBS as the original volume, 100 μL of each of yeast cultures was extracted and subjected to series dilution with a dilution factor of 10⁶ (a million-fold dilution). Then an aliquot (10 μL) of the 10⁶ dilution was plated out on nutrient agar. After 48 hours of incubation at 27° C., colonies formed were counted. Yeast survival rate was expressed as viability ratio to the control group.

The equation is shown below:

$$Yeast survival rate ( \% ) = \frac{Colony numbers of the experimental group}{Colony numbers of the control group} \%$$

### Bulk Extraction Efficiency Rate

The fermented media was adjusted to pH 2.0 using HCl and then autoclaved for 20 minutes to de-glucosidate glucosidic demethylated-nobiletins. The pH was then adjusted to 7.0 and the media was extracted twice. The first extraction was a vortex extraction using same process used for HPLC analysis. The second extraction was for bulk extraction.

Specifically, for the bulk extraction, 500 mL of the fermented culture was added to 100 mL extractant (hexane:ethyl acetate=4:6, v/v). This extraction was repeated for 4 times. After the bulk extraction, the media was extracted using a vortex extraction and HPLC analysis.

The bulk extraction efficiency rate in this research was calculated according to the following equation:

$$Bulk extraction efficiency ( \% ) = \frac{metabolite concentration before bulk extraction - metabolite concentration after bulk extraction}{metabolite concentration of media samples before bulk extraction}$$

### Statistics

Data presented herein are representative results from a set of more than three independent experiments or the mean±S.E.M. of those experiments. One-way ANOVA was used for all the statistical comparison among groups. A P-value of <0.05 was considered to be statistically significant.

### Results

Yeast Viability, Demethylation Efficiency and the Yield of Demethylated Nobiletins with Different Concentrations of DMSO/Nobiletin

Due to its hydrophobic nature, nobiletin should be dissolved in DMSO before being added into media for transformation. However, the maximal solubility of nobiletin in DMSO is not higher than 18 mg/mL. Meanwhile, high concentrations of both DMSO and nobiletin are toxic to yeasts. For example, DMSO can induce concentration-dependent cytotoxicity to cells (Kwak et al., 2010), bacteria and yeasts (Sadowska-Bartosz et al., 2013). Nobiletin can also decrease variability of yeast (Wang et al., 2018). To minimize the toxicity of DMSO and improve the availability of nobiletin as substrate for transformation, the concentration-dependent influences of DMSO/nobiletin were studied in terms of yeast variability, the yield of demethylated nobiletins as well as the transformation.

### Yeast Viability

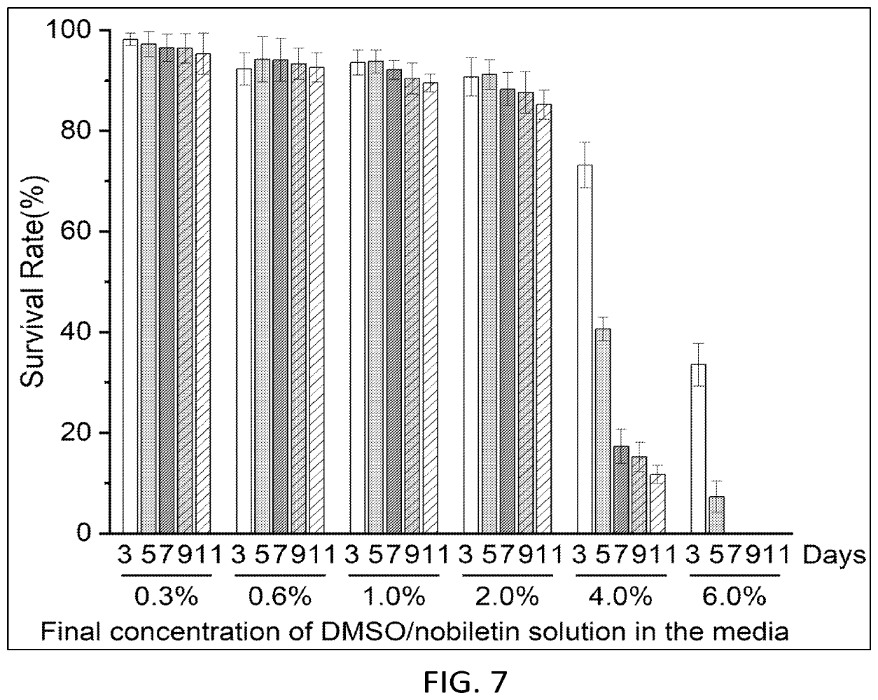

FIG. 7 demonstrates that as the concentration of DMSO/nobiletin increase from 0.3% to 2.0%, the survival rate showed constant decrease. For example, at concentration of 0.3% DMSO/nobiletin, the survival rate was 98.2±1.21% after 3 days of incubation. In contrast, at concentration of 2.0% DMSO/nobiletin, the survival rate was 90.7±3.8% after 3 days of incubation. However, after 11 days of incubation for all concentrations under 2.0%, the survival rate was not less than 80%. For example, at a concentration of 2.0% DMSO/nobiletin, the survival rate was 85.2±2.9% after 11 days. In other words, there was a concentration-dependent influence on the survival rate of the yeast. But the negative effect from DMSO/nobiletin was not destructive if the concentration was not higher than 2.0%.

However, when the DMSO-nobiletin concentration reached 4.0% or 6.0%, the survival rate was seriously inhibited. This influence became more prominent with longer incubation times. When the DMSO-nobiletin concentration was 4.0%, the survival rate was 73.2±4.5% after 3 days of incubation, 40.6±2.3% after 7 days of incubation and only 11.7±1.8% after 11 days. Additionally, the yeast could barely survive for 5 days when the concentration of DMSO-nobiletin system was 6.0%. Visually apparent cell lysis was caused by the 6% concentration (data was not shown).

Yee et al. (1972) investigated the effect of DMSO on the viability of Saccharomyces cerevisiae strain MCC. These investigators found that the exposure to increasing concentrations of DMSO results in rapidly decreasing cell viability above 20% (v/v) DMSO. Cytolysis occurred as the concentration of DMSO reached 40%. In our research, the DMSO/nobiletin concentration threshold turned out to be less than 4.0% which was much lower than 40%. This decrease is likely caused by either the differences in yeast species or the co-existence of nobiletin. The concentration of DMSO-nobiletin should be limited to 2.0% (nobiletin concentration <180 g/mL) to protect yeast viability.

### Demethylation Efficiency

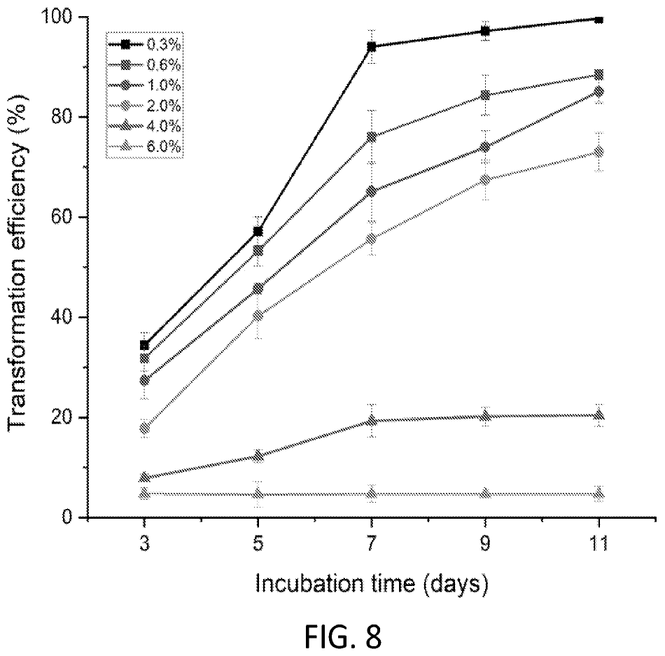

As shown in FIG. 8, there was a concentration-dependent influence on nobiletin transformation efficiency. Samples with 0.3% or 0.6% of DMSO/nobiletin enjoyed the highest transformation efficiency throughout the 11 days of incubation. Samples with 1.0% or 2.0% of DMSO/nobiletin also showed high transformation efficiency. Experimental groups with 6.0% DMSO/nobiletin had the lowest transformation efficiency. Samples with 4.0% DMSO/nobiletin also showed low transformation efficiency. Accordingly, increasing the concentration of DMSO/nobiletin showed increasingly detrimental effects on the transformation efficiency of the nobiletin.

This effect might be closely related with the survival rate as shown in FIG. 7. 0.3% and 0.6% DMSO/nobiletin exerted minimal effect on the survival rate (>95%); therefore, the active yeast could efficiently demethylate more than 97% of nobiletin after 11 days of incubation. 1.0% and 2.0% DMSO/nobiletin solutions induced a 10% to 15% decrease to the survival rate, thereby inhibiting the transformation efficiency as well. As shown in FIG. 8, the maximal transformation efficiency for samples with 1.0% or 2.0% DMSO/nobiletin were respectively 85.1±2.3% and 80.3±4.2%. Samples with 4.0% of DMSO/nobiletin, had a relatively high survival rate (>40%) within the first 5 days. It is likely that these active yeasts contributed to the approximate 20% transformation efficiency. In the last 6 days of incubation, despite ˜20% survival rate, the transformation efficiency showed no significant increase as the surviving yeasts lost the transformation ability. Samples with 6.0% DMSO/nobiletin showed 5.2±1.2% transformation efficiency after 3 days of incubation. No significant change was shown thereafter. This is likely due to the extremely low survival rate.

### The Yield of Demethylated-Nobiletins

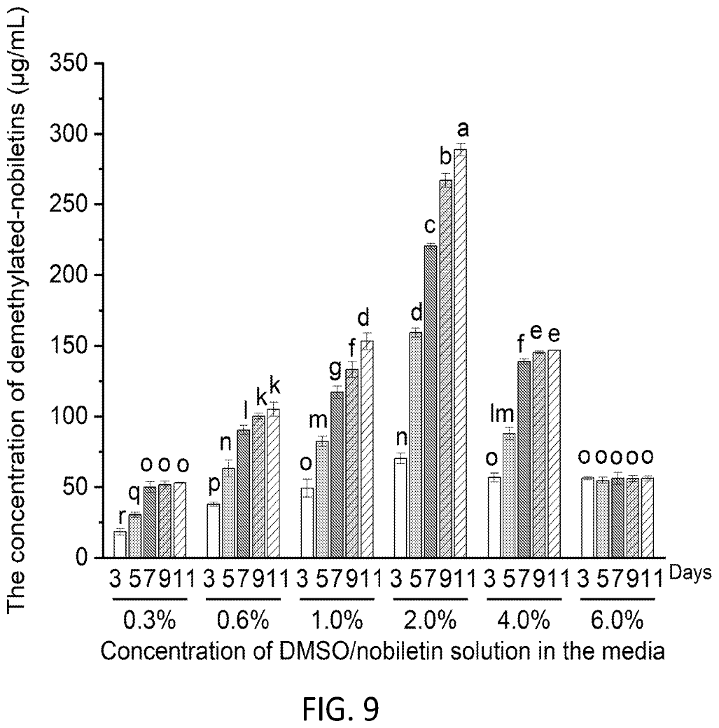

As shown in FIG. 9, samples with 2.0% of DMSO/nobiletin gave the highest yield of demethylated-nobiletins for each of the sampling time. For example, after 3 days incubation with 2% DMSO/nobiletin, a total of 70.3±3.6 μg/mL demethylated-nobiletins was achieved. This was significantly higher than the production of any other samples. Moreover, at 2% DMSO/nobiletin, the maximum yield (289±4.4 μg/mL) was achieved after 11 days of incubation.

Samples with 0.3% DMSO/nobiletin could only provide a yield around 50 μg/mL, even though they exhibited the highest survival rate and transformation efficiency. The limited accessibility of nobiletin clearly restricted the yield. In contrast, when the concentration of DMSO/nobiletin reached 6.0% (the highest accessibility of nobiletin), the yield remained low with the value being around 55.0±5.5 μg/mL. This was due to the low survival rate of yeast and the reduced transformation efficiency.

Accordingly, the yield of demethylated-nobiletins was directly related to the survival rate, transformation efficiency and substrate accessibility. In the DMSO/nobiletin system, 2.0% of DMSO/nobiletin provided the highest yield of PMF metabolites.

### HMS/Nobiletin Dispersion System

As shown above, the maximal yield of demethylated-nobiletins was around 290 μg/mL when the concentration of DMSO/nobiletin was set at 2.0%. However, because of the high concentration of DMSO, the bulk extraction efficiency was less than 60% (data was not shown). Meanwhile, DMSO is a hazardous compound to human body and the environment. Therefore, a green substitute nobiletin-HMS nano/macro-meter dispersion system was developed using the media-milling technology.

### Effect of the Media Milling Process on Particle Size of HMS/Nobiletin Dispersions

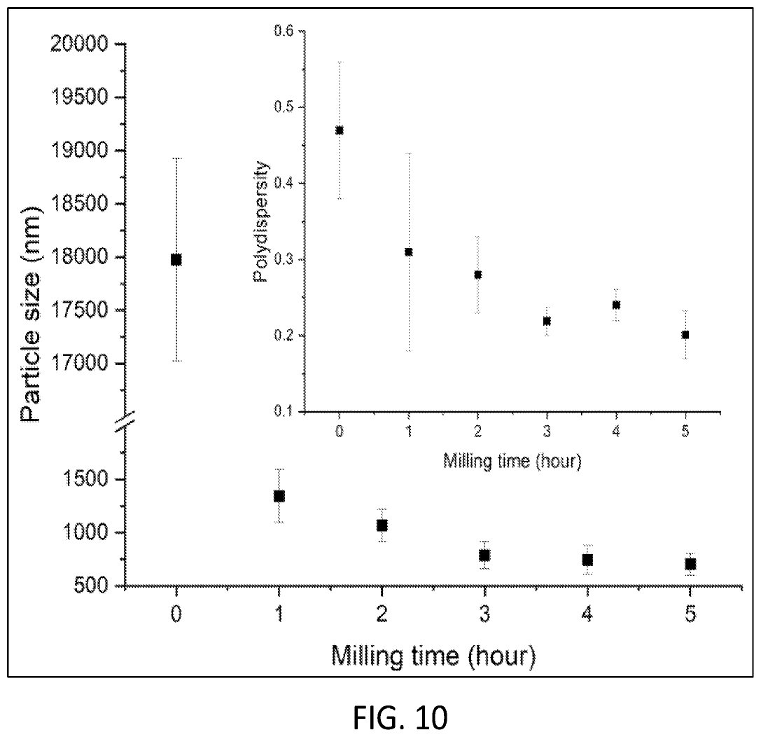

To produce HMS-nobiletin nano or macro-meter dispersions (HMS/nobiletin), a media milling technique was employed. This technique significantly decreased the particle size of dispersions. As shown in FIG. 10, the average particle size before media milling was 17975±950 nm. This was reduced to 1344±25 after 1 hour of media milling. The particle size continued to decrease and reached 788±57 nm after 3 hours of milling. However, in the following 2 hours of media milling, the particle size showed no significant decrease with the value being 745±60 nm and 703±93 nm respectively.

The reduction of the particle size was caused by two factors: the intense shear force during the media milling process and the hydrophobic features of nobiletin and HMS. The adsorption behavior between two hydrophobic molecules helped the stabilization of the ground mixture (Pongpeerapat, Wanawongthai, Tozuka, Moribe, &Yamamoto, 2008). Therefore, the hydrophobic region of HMS absorbed to the lipophilic surface of dispersed nobiletin nano or macro-meter particles in the media milling process, and subsequently reduced the contact between nobiletin themselves. In summary, the media milling technique together with the application of HMS effectively reduces the particle size of the nobiletin-HMS dispersion.

### Solubility Studies

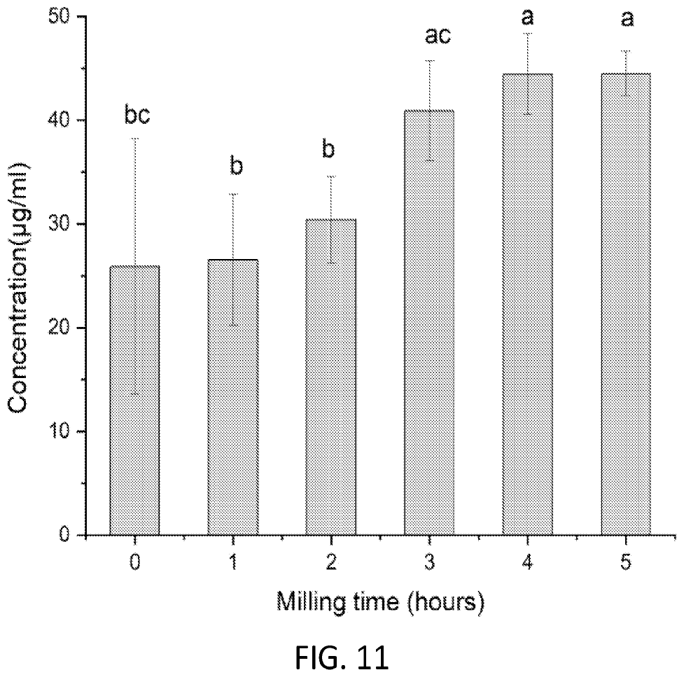

To check the water solubility of nobiletin in the nobiletin-HMS dispersion system, an excess of the media-milled nobiletin powder was dispersed into distilled water and agitated thoroughly for 24 hours at room temperature to reach full saturation. The solubility of nobiletin dispersions with different milling time is shown in FIG. 11. As the milling time increased from 0 hour to 2 hours, the average solubility of nobiletin raised from 25.9 μg/mL to 30.4 μg/mL. When the milling time reached 3 hours, an increase in the nobiletin solubility to 40.9±4.8 μg/mL was observed. The solubility increased further to 44.4±3.9 μg/mL after 4 hours of milling. Compared with the pure nobiletin's water solubility of 9.2 μg/mL at 25° C., this dispersion system with 3 hours of milling can enhance the water solubility of nobiletin by approximately five-fold.

Yeast Viability, Demethylation Efficiency and the Yield of Demethylated Nobiletins with Different Concentrations of HMS/Nobiletin

Nano- or macro-meter HMS/nobiletin formula can be successfully constructed with 4 hours of media milling process. This formula described above improved the water solubility of nobiletin by 5 times. To optimize the amount of HMS/nobiletin, transformation, yeast viability, demethylation efficiency and the yield of demethylated nobiletins were evaluated at different concentrations.

### Yeast Viability

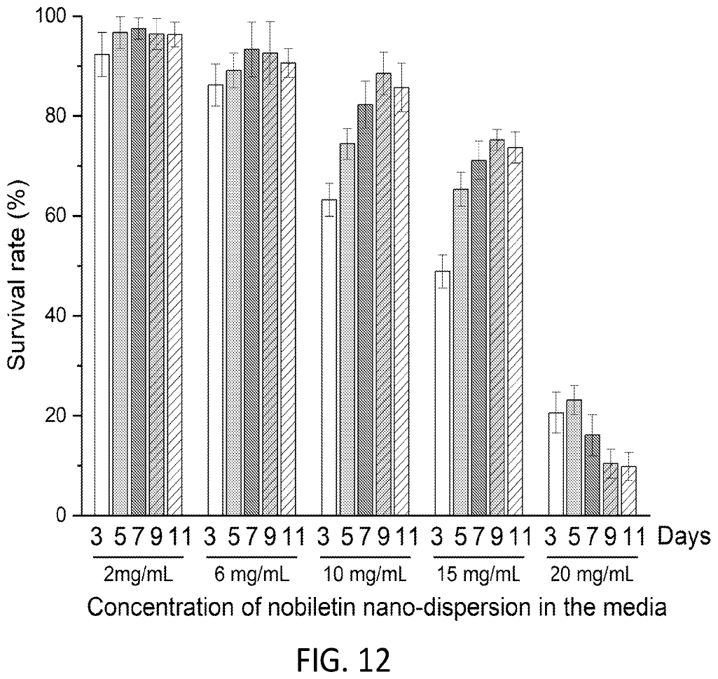

FIG. 12 demonstrates that yeast survival rate decreased as the concentration of HMS/nobiletin in the media increased. For example, the survival rates of samples with 2 mg/mL of HMS/nobiletin were about 90% after 11 days of incubation. In contrast, at an HMS/nobiletin concentration of 15 mg/mL, the survival rates were about 80%. Furthermore, the survival rates fell to the range of 10% to 22% when the concentration reached 20 mg/mL.

Additionally, survival rates were also dependent on the incubation time. After 11 days of incubation, samples with 2 mg/mL of HMS/nobiletin had a survival ratio at around 98%. However, survival rates of samples that had 6-15 mg/mL of HMS/nobiletin increased in the first 5-7 days of incubation and then showed no significant change. Interestingly, the survival rates of samples with 20 mg/mL of HMS/nobiletin continued to decrease after the 5th day of incubation.

Microorganisms need time to adapt themselves to a new environment. The impact of 2 or 6 mg/mL HMS/nobiletin was very small, and therefore allowed the yeasts to duplicate very quickly and to enjoy an approximate 95% or 90% survival rate. With the increasing concentration of HMS/nobiletin, the toxicity of high concentrations of nobiletin became more and more prominent. Additional time was provided for the yeast to adapt to the environment in these conditions. This adaptation time in samples with 10-15 mg/mL HMS/nobiletin (nobiletin 625-937.5 μg/mL) caused an initially low survival rate that increased to a high level after several days of incubation as shown in FIG. 12.

Notably, when compared with the DMSO/nobiletin system, HMS/nobiletin dispersions allowed the same or higher survival rate at higher concentrations of nobiletin. For example, samples with 6 mg/mL HMS/nobiletin (nobiletin 375 μg/mL) exhibited a survival rate range of 86.2±4.2% to 93.3±5.5%. However, the survival rates for samples that had 2.0% DMSO/nobiletin (nobiletin 360 μg/mL) had a survival rate range from 85.2±2.9% to 90.7±3.8%.

Moreover, after 7 days of incubation, samples with 4.0% DMSO/nobiletin (nobiletin 720 g/mL) only had survival rates less than 40%, but samples with 15 mg/mL HMS/nobiletin (nobiletin 937.5 μg/mL) enjoyed survival rates higher than 70%.

Accordingly, when compared with the DMSO/nobiletin system, the HMS/nobiletin dispersion was less toxic to the yeast, which made it possible to improve the availability of nobiletin for transformation and resulting yield.

### Demethylation Efficiency

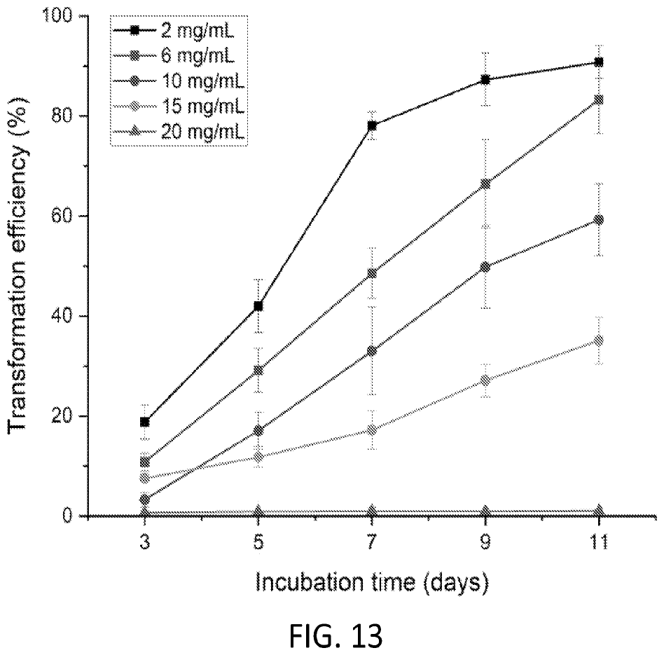

The nobiletin transformation efficiency was studied as a function of incubation time and HMS/nobiletin concentration. As showed in FIG. 13, most samples showed increasing transformation efficiency as incubation time increase. Only the 20 mg/mL HMS/nobiletin sample showed little to no change. However, the increasing rate and the maximum transformation efficiency of each sample varied. For example, the transformation efficiency at 6 mg/mL HMS/nobiletin increased from 35.0±2.8 μg/mL in the 3rd day to 97.3±1.1 μg/mL in the 11th day. In contrast, the transformation efficiency at 15 mg/mL HMS/nobiletin increased from 1.8±0.6 μg/mL to 26.60±4.16 μg/mL. In conclusion, there was a negative dose-response effect of HMS/nobiletin on the transformation efficiency. Accordingly, increasing the concentration of HMS/nobiletin showed increasingly detrimental effects on the transformation efficiency of the nobiletin.

Compared with the DMSO/nobiletin system, HMS/nobiletin dispersions showed lower transformation efficiency. The transformation efficiency of samples that had 0.3% to 2.0% DMSO/nobiletin were higher than 80% after 11 days of incubation, while the transformation efficiency of samples with HMS/nobiletin dispersions was generally lower than 80%. This might be due to the difference in homogeneity between the systems.

### The Yield of Demethylated-Nobiletins

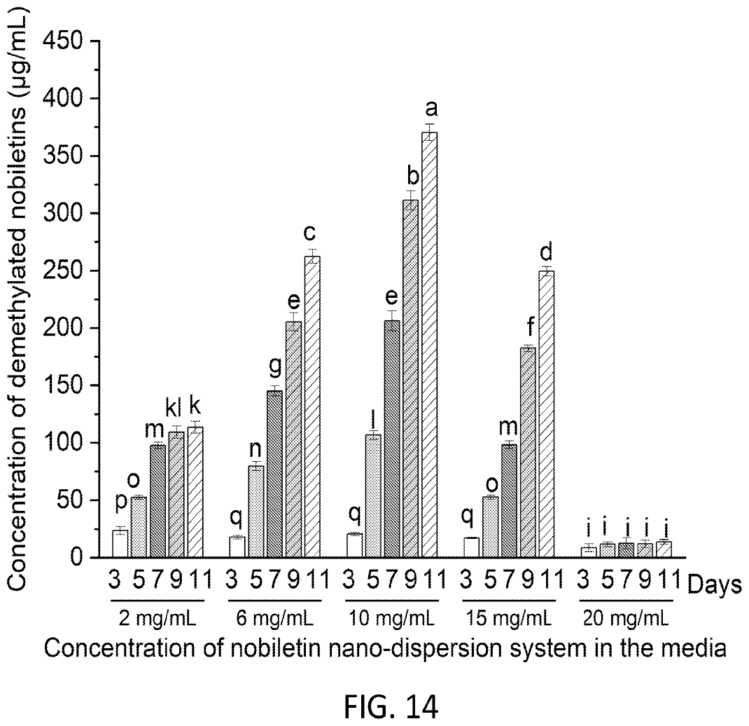

As shown in FIG. 14, in HMS/nobiletin concentrations ranging from 6 mg/mL to 15 mg/mL, the yield of demethylated-nobiletins increased with the incubation time. But in each sample, a concentration of 10 mg/mL HMS/nobiletin had the highest production. After 11 days of incubation, the yield of demethylated-nobiletins was approximately 370.4±7.2 μg/mL.

Notably, the yield was around 1.5 times of the highest yield that was achieved when DMSO/nobiletin concentration was 2.0%, despite of the relatively lower transformation efficiency (FIG. 13). The increased yield is due to the higher nobiletin availability (625 μg/mL) provided by the 10 mg/mL HMS/nobiletin.

### Bulk Extraction Efficiency Rate

DMSO is a popular amphiphilic small molecule which is composed of one hydrophilic sulfoxide group and two hydrophobic methyl groups. The amphiphilic nature would inhibit the movement of target compounds from the aqueous phase to the organic solvent phase during extraction. Hydrophobically modified starch (HMS) belongs to big molecule with amphiphilic feature. However, after the autoclave process, HMS would be denatured and could be removed by centrifugation, which prevents its interference in the extraction efficiency. Therefore, the bulk extraction efficiency rates by using DMSO/nobiletin solution or HMS/nobiletin dispersion in the fermentation system was compared.

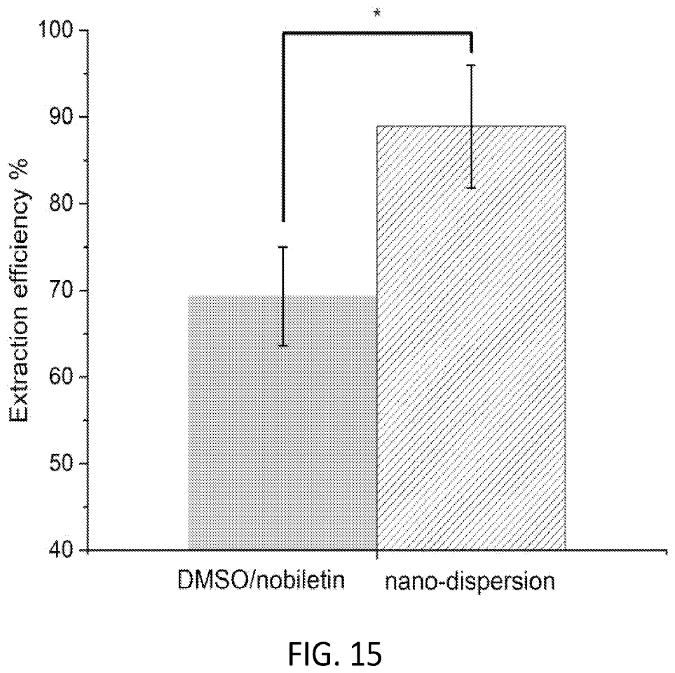

As shown in FIG. 15, the bulk extraction efficiency by using HMS/nobiletin dispersion was around 90%. In contrast, the extraction efficiency was at 70% when the DMSO/nobiletin solution was employed. Therefore, the nano-dispersion system is a better choice.

Nano- or macro-meter HMS/nobiletin formula which could improve the water solubility of nobiletin was successfully constructed with the media milling technique.

Both HMS/nobiletin nano-dispersion and the DMSO/nobiletin solution exerted concentration-dependent negative effects on the yeast viability and transformation efficiency. However, the yield of dimethyl-nobiletins was affected by the demethylation efficiency and the substrate availability.

In comparison, the HMS/nobiletin nano-dispersions system was less toxic to the yeast, and therefore made it possible to improve the availability of nobiletin for transformation. Consequently, HMS/nobiletin dispersions achieved higher yield of dimethyl-nobiletins than the DMSO/nobiletin system, despite of the fact that it showed a lower transformation efficiency. Additionally, the bulk extraction efficiency was much higher when the HMS/nobiletin was used.

### REFERENCE

Bier, M. C., A. B. Medeiros, and C. R. Soccol. (2017). Biotransformation of limonene by an endophytic fungus using synthetic and orange residue-based media. Fungal Biol, 121(2): p. 137-144.
Pongpeerapat, A., Wanawongthai, C., Tozuka, Y., Moribe, K., & Yamamoto, K. (2008). Formation mechanism of colloidal nanoparticles obtained from probucol/PVP/SDS ternary ground mixture. International Journal of Pharmaceutics, 352(1-2), 309-316.
Pralhad, T., & Rajendrakumar, K. (2004). Study of freeze-dried quercetin-cyclodextrin binary systems by DSC, FT-IR, X-ray diffraction and SEM analysis. Journal of pharmaceutical and biomedical analysis, 34(2), 333-339.
Sadowska-Bartosz, I., Pgczka, A., Molon, M., & Bartosz, G. (2013). Dimethyl sulfoxide induces oxidative stress in the yeast Saccharomyces cerevisiae. FEMS yeast research, 13(8), 820-830.
Silva, C. D., Herdeiro, R. S., Mathias, C. J., Panek, A. D., Silveira, C. S., Rodrigues, V. P., . . . & Nogueira, F. L. P. (2005). Evaluation of antioxidant activity of Brazilian plants. Pharmacological research, 52(3), 229-233.
Yeh, A. I., Huang, Y. C., Chen, S. H. (2010). Effect of particle size on the rate of enzymatic hydrolysis of cellulose. Carbohydrate Polymers, 79(1): p. 192-199.
Wang, M., Meng, D., Zhang, P., Wang, X., Du, G., Brennan, C., . . . & Zhao, H. (2018). Antioxidant protection of nobiletin, 5-demethylnobiletin, tangeretin, and 5-demethyltangeretin from citrus peel in Saccharomyces cerevisiae. Journal of agricultural and food chemistry, 66(12), 3155-3160.

### Example 3

### Treatment of Lipogenesis with M76N

Abundant research has shown that 3″-4′,′, or 5′-monodemethyl-nobiletin metabolites exhibit significant pharmacological activities. Despite this research, no report ever reveals the bio-beneficial properties of any 6- or 7-monodemethyl nobiletin. Herein we provide data exhibiting the anti-adipogenesis properties of these metabolites of nobiletin.

### Materials and Methods

Dulbecco's modified Eagle's medium (DMEM high-glucose), and penicillin/streptomycin antibiotic mixture were obtained from Life Technologies, Inc. (Grand Island, NY, USA). Fetal bovine serum was obtained from Moregate BioTech (Bulimba, Australia). Insulin, dexamethasone, 3-isobutyl-1-methylxanthine, and Oil Red O were obtained from Sigma (St. Louis, MO, USA).

Primary antibodies which include AMPK-amAb, and Phospho-AMPKa(p-AMPKa) (Thr172) mAb, Acetyl-CoA Carboxylase (ACC) mAb, fatty acid synthase mAb RAs-related Nuclear protein (Ran) pAb, PPARc mAb, and C/EBPa pAb were all obtained from Cell Signaling Technology (Danvers, MA, USA). They were diluted by 1000 times for immunoblotting analyses.

### Cell Lines and Culture Conditions

3T3-L1 cells were purchased from American Type Culture Collection (ATCC, Rockville, MD) and maintained in MEME media with 10% FBS 1% penicillin/streptomycin (GIBCO, Paisley, UK) in a humidified atmosphere of 5% CO₂ in air at 37° C. Cells were passaged by using 0.05% (vol) Trypsin and replated in 55-cm² dishes at a density of 1*10⁴ cells/cm² until confluence.

Two days after confluence, designated as day 0, adipocyte differentiation was initiated by culturing the cells for three days in differentiation medium (DM) which contained 10 μg/mL insulin, 0.5 mM IBMX, and 1 μM dexamethasone in complete culture medium. At day 3, the medium was replaced with DMEM, containing 10 μg/mL insulin. The medium was changed every two days until day 8. Cells were harvested on day 3 or day 8.

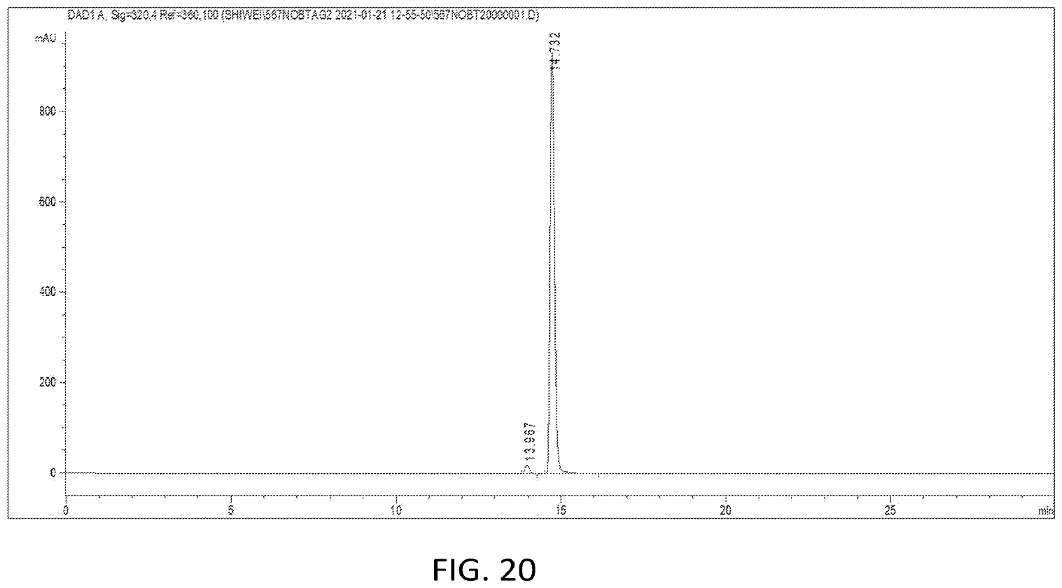

For treatment, nobiletin and the mixture of 6- and 7-OH-nobiletin (M67N) in a 1:49 ratio were first dissolved in dimethyl sulphoxide (DMSO) to make stock solution (5 mM to 100 mM). (FIG. 20) The stock solution was then diluted by 1000 times with culture medium to designated concentrations. Cells cultured in full DMEM was set as negative control while cells treated with DM with 0.1% DMSO as positive control.

### MTT Assay

3T3-L1 cells were seeded on 96-well plate at density of 1×10⁴ cells/well and incubated overnight for cell attachment. For measuring pre-confluent cell proliferation, cells were cultivated for 36 hours and full MEME with or without nobiletin or M67N was used. For measuring post-confluent cell proliferation during first three days of differentiation, cells were switched to DM with different concentrations of nobiletin or M67N for 72 hours. At the end of each treatment, cells were washed with PBS three times. MTT (100 L) was added at concentration of 0.5 mg/mL to the cells. After a 3-hour incubation, the medium was decanted from each well and 150 μL DMSO was added to dissolve the formed formazan crystals on a shaker. When the crystal was completely dissolved, the absorbance at 570 nm was measured. Cell viability was interpreted as the percentage (%) of the readout of treated cells divided by that of control. Six repeats were carried out for each concentration.

### Flow Cytometry Analysis

3T3-L1 cells were seeded in six-well plates at a density of 3-5×10⁵/well. After 2 days of confluency, cells were treated with DM in the absence or presence of several doses of nobiletin or M67N for 24h. Both detached and adherent cells were harvested by trypsinization and washed with phosphate buffered saline (PBS). Cells were fixed with 70% ice cold ethanol and placed at 4° C. overnight. After removal of ethanol, cells were stained with propidium iodide containing RNAse ((Carlsbad, CA)) for 30 min in the dark. Fluorescence cells analysis was carried out by using an Accuri C6 flow cytometer. Data were processed using Flowjo_10 software. Each experiment was performed 3 times to reduce variance.

### Oil Red O Staining

On day 8, cell layers were washed with 1×PBS and fixed with 10% paraformaldehyde (Sigma, St. Louis, MO, USA). After 15 minutes' fixation, the medium in each well was decanted and placed in a fume hood for complete dryness. Afterwards, cells were subjected to Oil Red O staining for 30 min, which was followed by immediate rinse in water. The intracellular lipid droplets were checked and pictures were captured on Olympus FSX100 Microscope. For the quantification of intracellular lipids, dyes were dissolved in 4% NP-40/isopropanol and the absorbance was measured at 520 nm by a microplate reader.

### Statistics

Data are presented as the means±SD. One-way ANOVA was used for all the statistical comparison among groups.

### Results

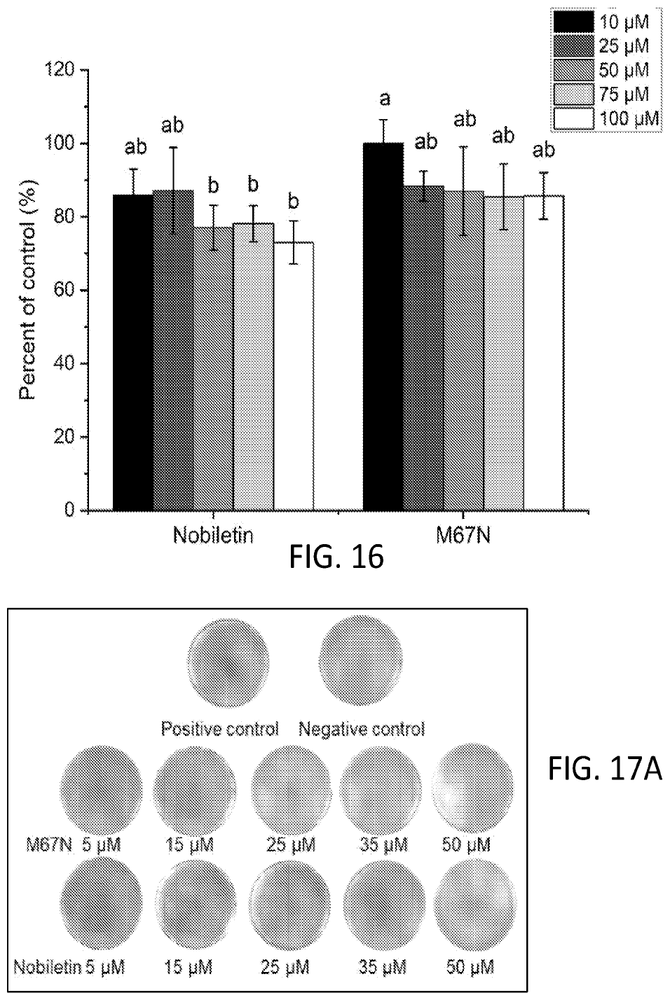

To access the safe concentration range of nobiletin, and its metabolites, the viability of pre-confluent 3T3-L1 preadipocytes were evaluated in the presence or absence of different doses of nobiletin and M67N. As shown in FIG. 16, nobiletin did not show any inhibitory effect on the proliferation of preadipocytes up to 75 μM. In contrast, when the concentration of nobiletin reached 100 μM, cell viability was reduced by 50%. M67N did not significantly alter cell proliferation even at 100 μM. Accordingly, the M67N metabolites are safer than nobiletin to 3T3-L1 preadipocytes.

M67N is More Potent than Nobiletin in Reducing Lipid Accumulation in Differentiating 3T3-L1 Adipocytes

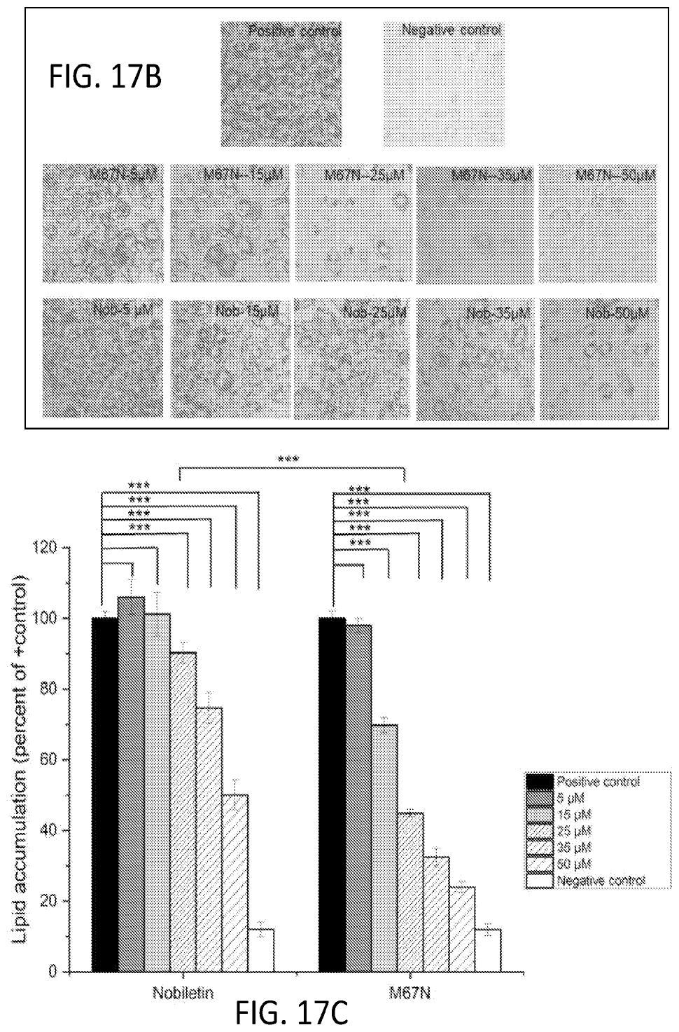

To compare the anti-adipogenic efficacy of nobiletin, and M67N, 3T3-L1 preadipocytes were induced by DM to differentiate in the presence of various concentrations of nobiletin or M67N, and cells were stained with Oil Red O solution. As shown in FIG. 17A, the positive control was dyed in red while the negative control showed no red color. Meanwhile, FIG. 17B shows that millions of intracellular lipid droplets formed in the positive control while no droplets were seen in the negative control group. This phenomenon proved that the preadipocytes were successfully differentiated into mature adipocytes characterized by intracellular macrovasiculor steatosis. The lipid accumulation was quantified by Oil Red O staining. As shown in FIG. 17C, both nobiletin and M67N decreased the intracellular lipid content in a dose-dependent way. Interestingly, M67N showed stronger potency in each concentration level. For example, when the concentration was 15 μM, nobiletin did not significantly lower lipid accumulation with the value being 101.254±6.096%; however, M67N reduced intracellular lipid content to 69.845±2.1%. At dose of 50 μM, M67N reduced lipid formation by more than 75% while nobiletin only inhibited 50% of lipid accumulation.

### N67M Suppressed the Differentiation of 3T3-L1 Cells in the Early and Late Phases of Adipocyte Maturation

In the process of adipocyte maturation, 3T3-L1 preadipocytes undergo multiple morphologic and genetic changes. The adipogenesis pathway of the cells is composed of two phases, an early phase from day 0 to day 3 and a late phase from day 4 to day 8 (Tomiyama, Nishio & Watanabe, 1999; Inokawa et al., 2016)

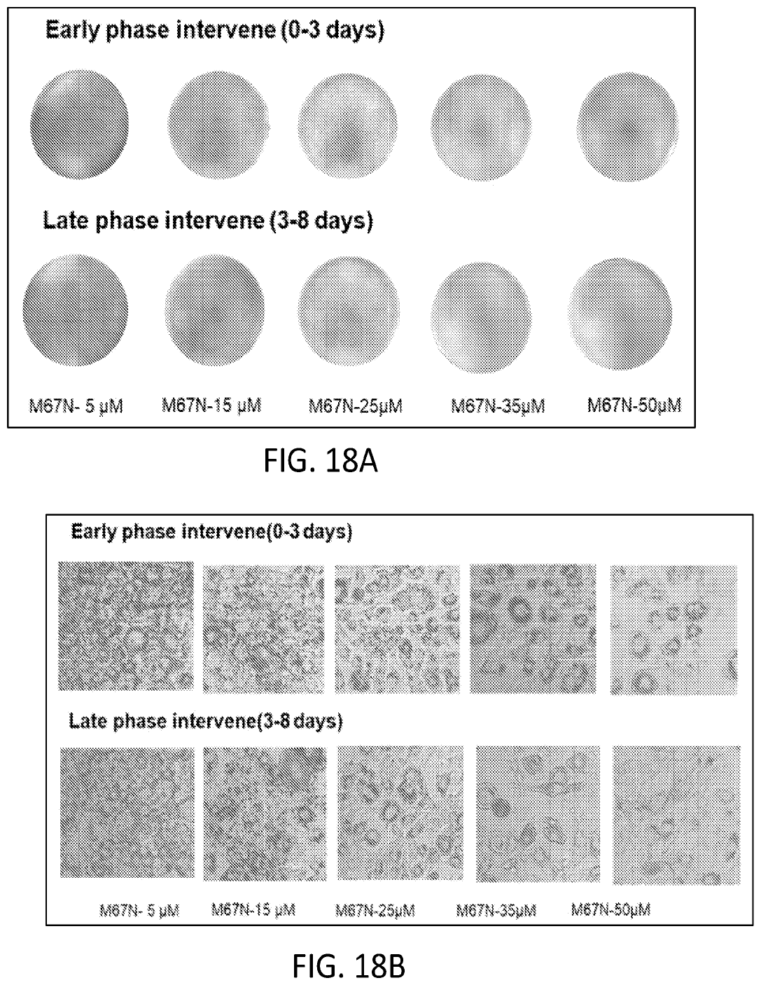

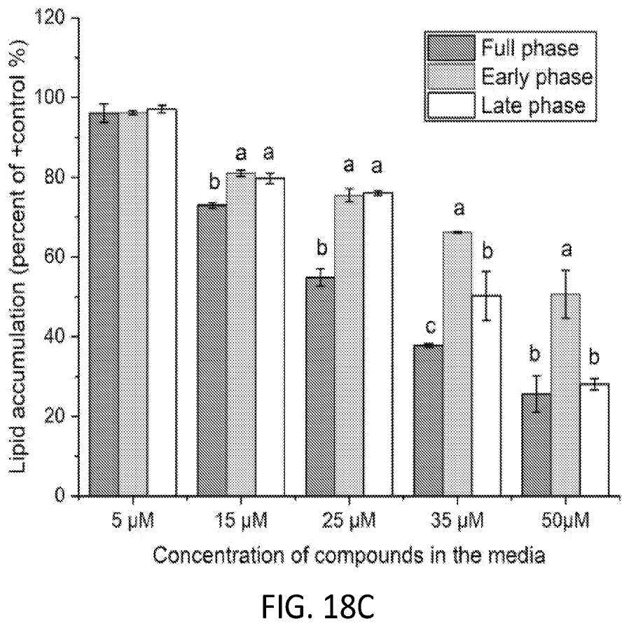

To test the effects of M67N on each phase, we treated the cells with M67N at different phases: early phase treatment (day 0 to day 3), late phase treatment (day 4 to day 8) and full phase treatment (day 0 to day 8) (FIG. 18A). As shown in FIG. 18B, both the early phase treatment and the late phase treatment suppressed the lipid accumulation in a dose-dependent matter. However, the effectiveness of each of the early phase and late phase treatment was weaker than the full phase treatment. These findings indicate that M67N inhibited andipogenesis by intervening intracellular events which run in parallel with clonal expansion. M67N also exhibited effects on the terminal differentiation process. FIG. 18 shows that when the concentration of M67N was at 15 μM and 25 μM, the anti-andipogenesis capacity of early-phase and late-phase treatments were comparable. However, as the dose increased, late phase treatment presented stronger suppressive activity than the early phase treatment. For example, at 50 μM, late-phase treatment inhibited lipid accumulation by 72%. These results are comparable with full phase treatment. In contrast early phase treatment decreased lipid content by 50%.

### Effects of M67N on Post-Confluent Mitotic Clonal Expansion and Cell Cycle Progression of 3T3-L1 Cells During the Early Stage of Differentiation

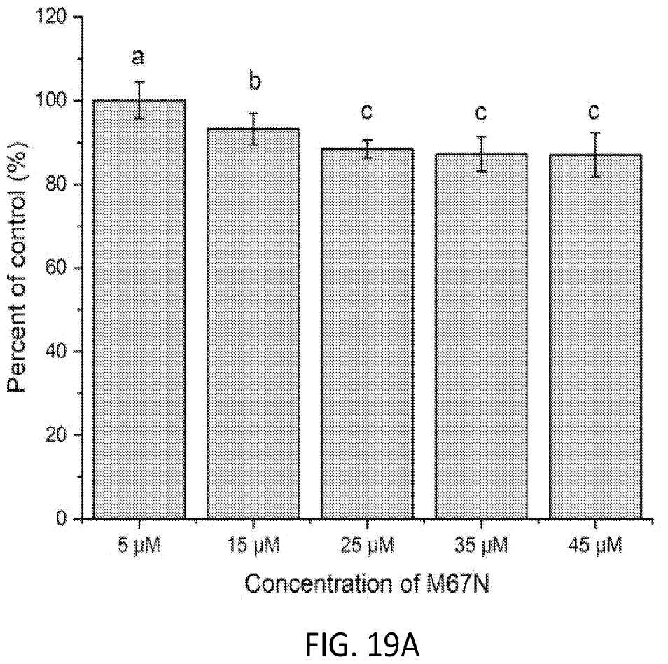

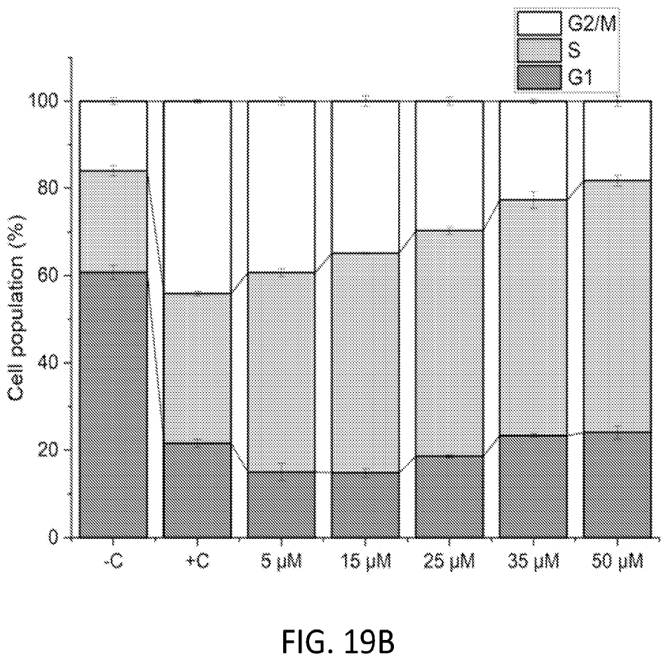

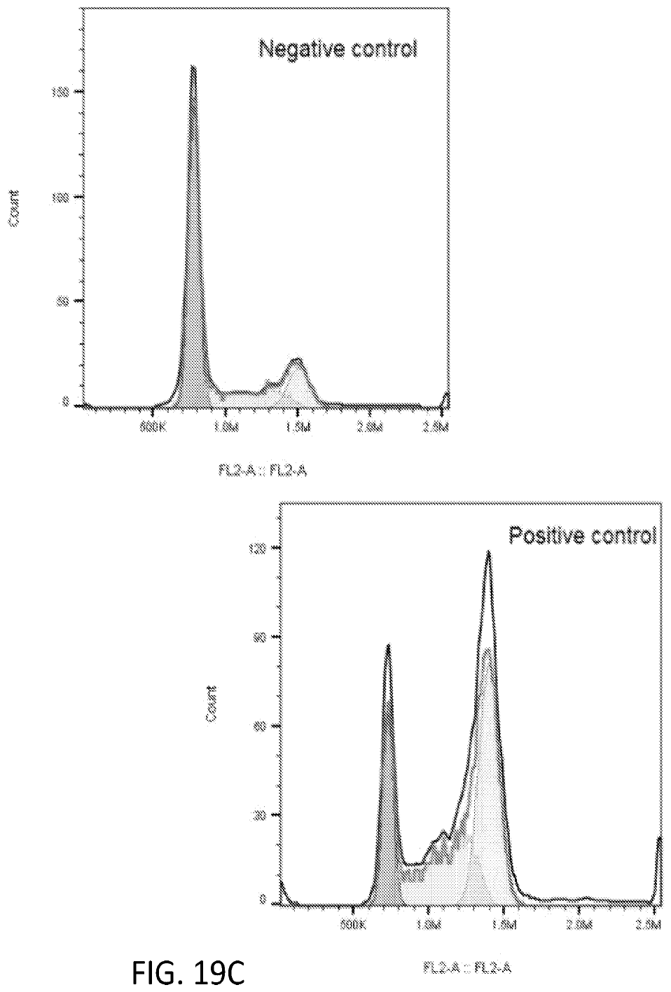

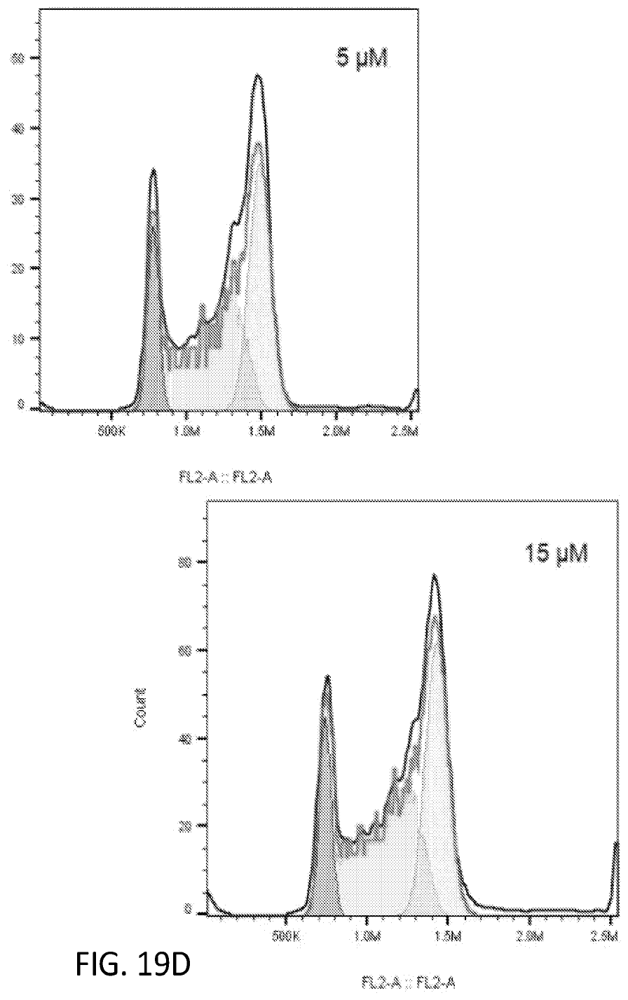

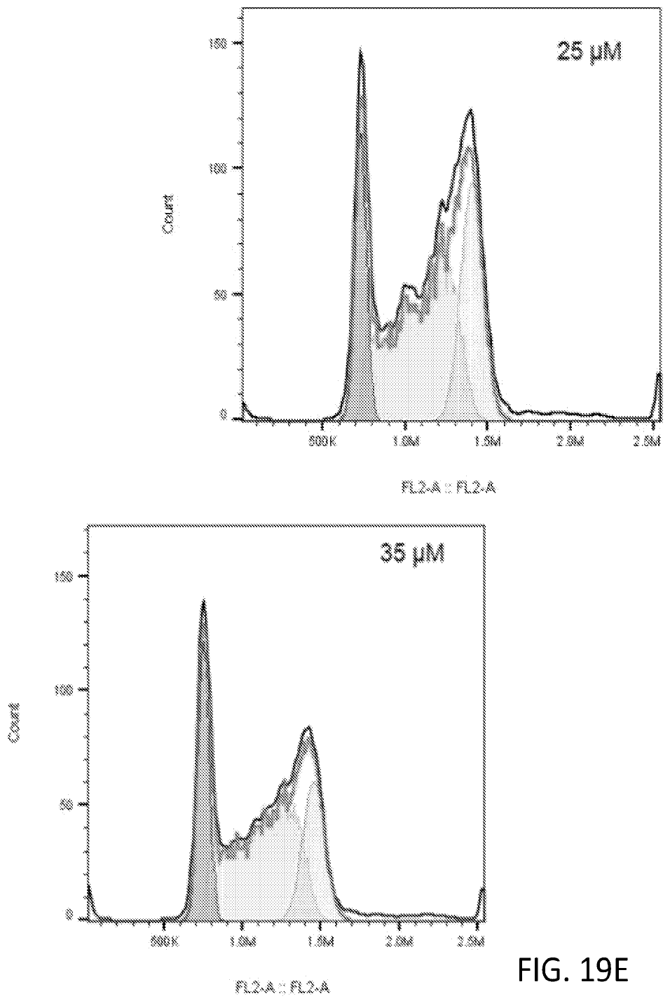

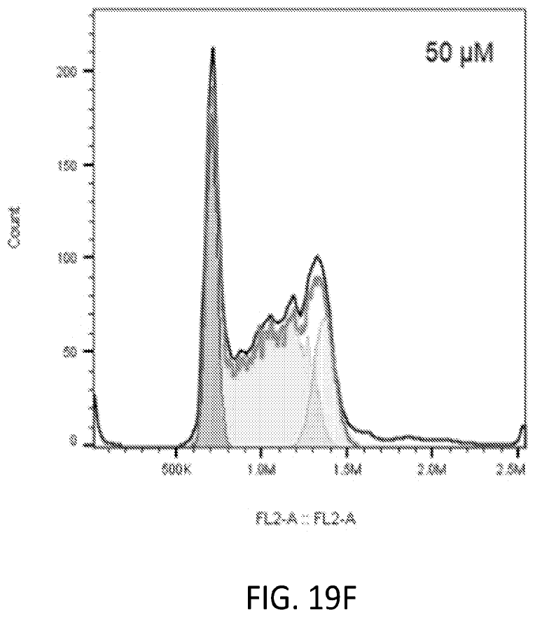

After exposure to differentiation inducers, post-confluent 3T3-L1 cells would exit growth-arrest and undergo mitotic clonal expansion (MCE) during the first 72 hours prior to terminal differentiation (Tang Q-Q, Otto TC, Lane DM., 2003). MCE plays a critical role in the adipocyte differentiation process, which means blocking DNA replication could prevent differentiation (Patel Y M, Lane, 2000). Since early phase treatment markedly suppressed adipogenesis, M67N could affect the preadipocyte proliferation step. A MTT assay which reflects the total number of living cells was performed on post-confluent cells. As demonstrated in FIG. 19A, M67N at 5 μM did not affect cell viability. With increasing doses from 15 M and 25 PM, cell viability decreased to 93.24±3.72 and 88.36±4.08 respectively. At increasing concentrations, viability remained at approximately 87% viability.

Next, flow cytometry with PI staining was performed to analyze cell cycle profiles of treated and untreated cells. FIG. 19B shows that NM67N significantly delayed the progression of the cell cycle and increased G0/G1 and S population in a dose-dependent manner. Some reported compounds, such as apigetrin, were able to exhibit its strongest effects during the early stages of differentiation. For example, apigetrin could arrest 3T3-L1 preadipocytes at G1 phase and significantly decrease cell viability by 50% (Hadrich & Sayadi, 2018). However, the population of cells at G1 phase described herein was low, when compared with the negative control group, even after the treatment of 50 μM of M67N. This result was consistent with the weak influence of M67N on the proliferation of post-confluent 3T3-L1 cells. Meanwhile, it explained the results that the early phase treatment suppresses the lipid accumulation but with weaker efficacy than late phase treatment.

### Example 4

Treatment of Lipogenesis with 6-OH-Nobiletin and 7-OH-Nobiletin Materials and Methods

Except as expressly contradicted herein, the materials and methods provided in Example 3 were used herein to test the treatment of lipogenesis with 6-OH-nobiletin and 7-OH-nobiletin.

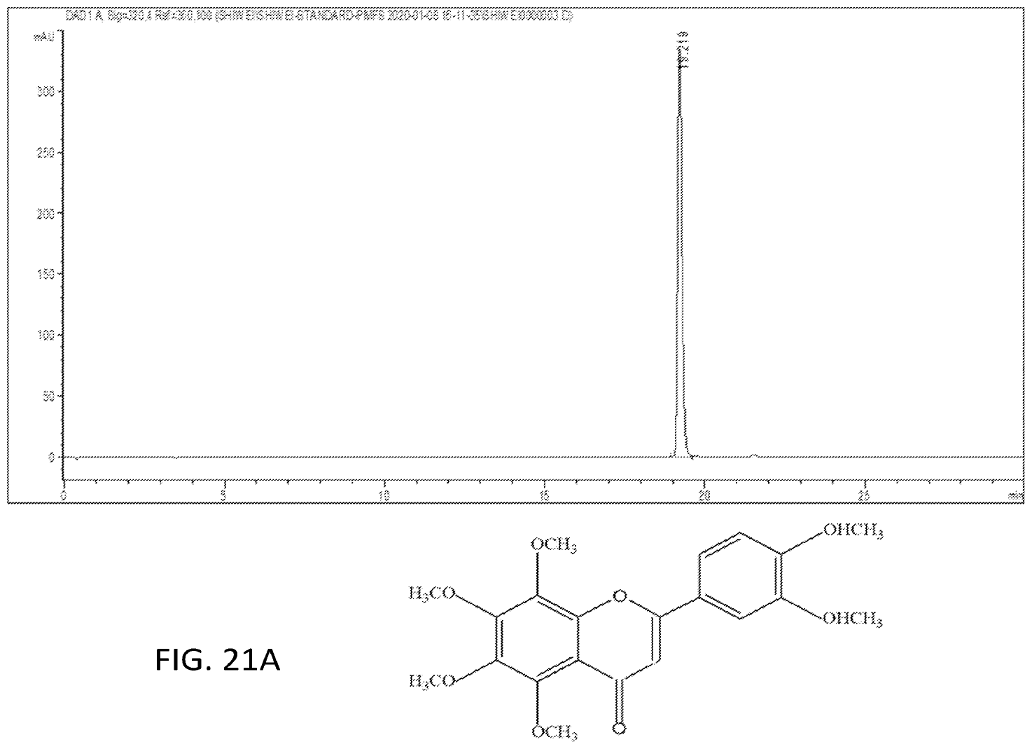

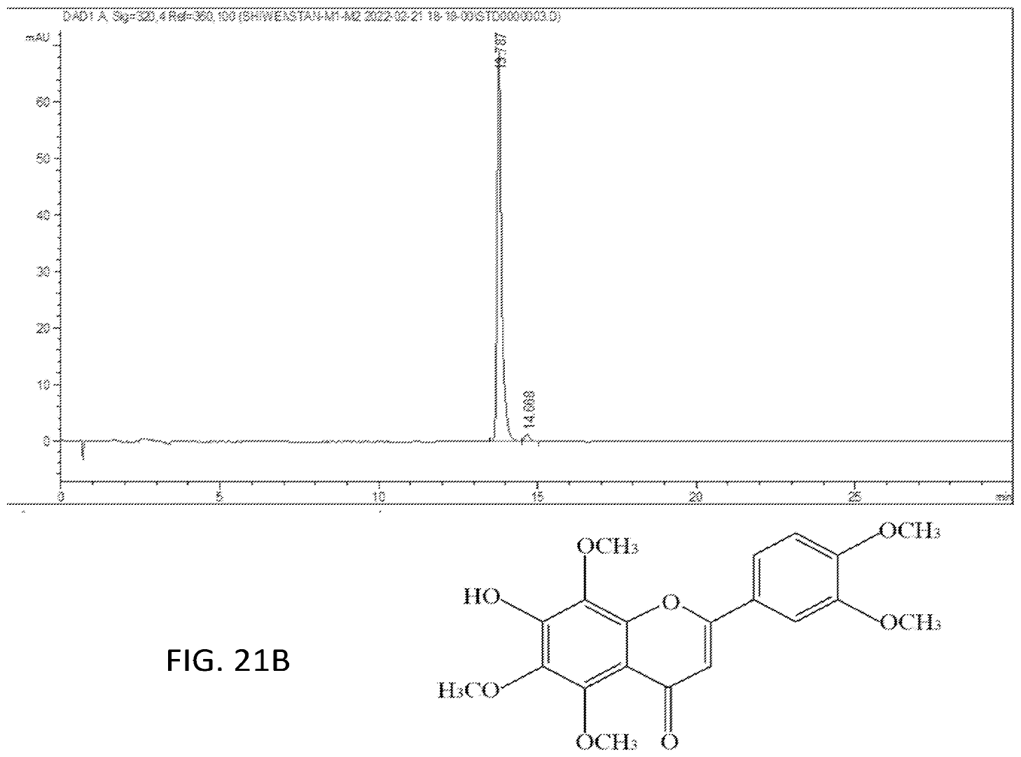

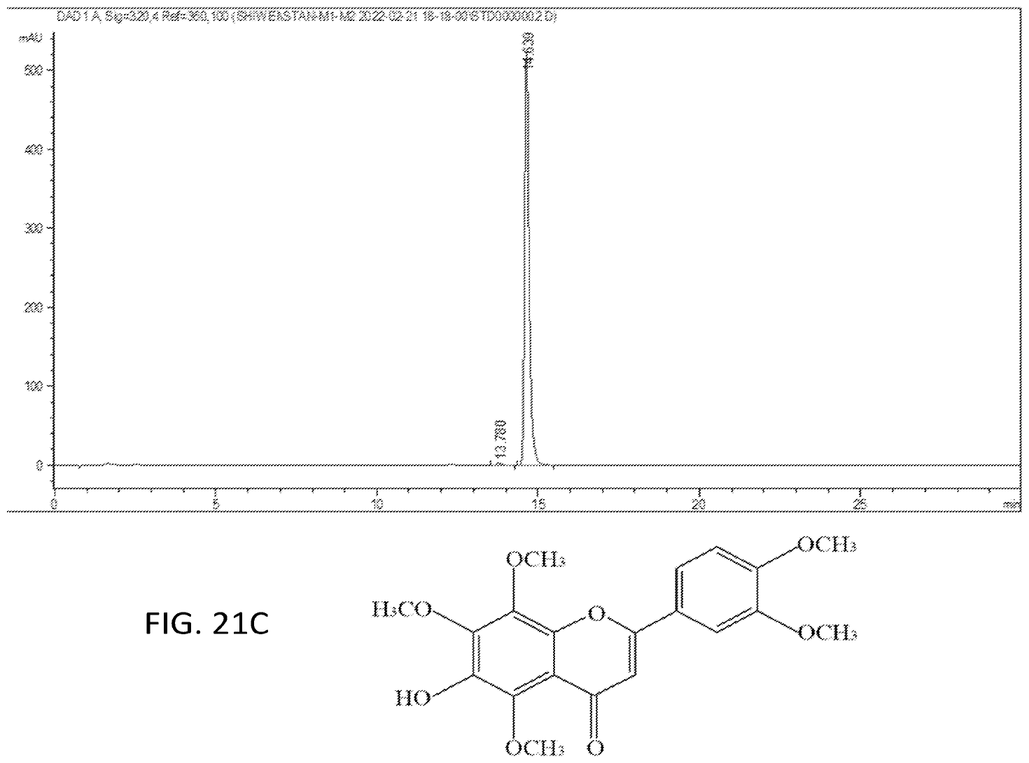

Nobiletin was purchased from Chengdu Runde pharmaceutical CO., LTD, Chengdu, China. 6-OH-nobiletin and 7-OH-nobiletin were produced through the procedures as described in the Examples above. FIG. 21A-21C presents the HPLC profile and chemical structure of nobiletin, 6-OH-nobiletin, and 7-OH-nobiletin.

### MTT Assay

3T3-L1 cells were seeded on 96-well plate at density of 1×10⁴ cells/well and were incubated overnight for cell attachment. For measuring pre-confluent cell proliferation, full MEME with or without nobiletin or 6-OH-nobiletin, or 7-OH-nobiletin was used and cells were cultivated for 36 hours. For measuring post-confluent cell proliferation during the first three days of differentiation, cells were treated after 2 days of confluence. Specifically, cells were switched to differentiation medium (DM) with different concentrations of targeted compounds for 24 hours. At the end of each treatment, cells were washed with PBS three times followed by the addition of 100 μL MTT at concentration of 0.5 mg/mL. After 3 hours' incubation, the medium in each well were decanted while 150 μL DMSO was added to dissolve the formed formazan crystals at a shaker. When the crystals were completely dissolved, the absorbance of at 590 nm was measured. Cell viability was interpreted as the percentage (%) of the readout of treated cells divided by that of control. Five repeats were carried out for each concentration.

### Protein Extraction and Immunoblotting Analysis

At day 3 or day 8, cells are harvested with scraper and homogenized with lysis buffer containing 0.5 mM PMSF, 1 mM mini-complete protease inhibitor cocktail (Sigma, St. Louis, MO, USA) and phosphatase inhibitor cocktail (Sigma, St. Louis, MO, USA). Lysates are syringed with 26 gauge ½ in. needles on ice and then applied for centrifugation at 14,000g for 15 min at 4° C. Protein concentration of cell lysates are determined by using Protein and Protein Broad Range (BR) Assay Kits (Thermofisher, Waltham, MA USA) according to the venture's protocol. Lysates are then stored at −80° C. or immediately are subjected to sodium dodecyl sulfate polyacrylamide gel electrophoresis (SDS-PAGE). For the SDS-PAGE, cell lysates are mixed with loading buffer and heated at 95° C. for 5 min prior to electrophoresis. 10% tris-glycine mini gels (Life Technologies, Carlsbad, CA, USA) are used for analysis of AMPK, p-AMPK, RAN (RAs-related nuclear protein), PPARα, C/EBPγ and FAS while 7.5% tris-acetate mini gel is used for ACC (acetyl-CoA carboxylase). 10% tris-glycine mini gel (Life Technologies, Carlsbad, CA, USA) is for analysis of FAS (fatty acid synthase). For immunoblotting, proteins are transferred to polyvinylidene difluoride membranes (Millipore, Belgium) at 4° C. for 2-3 hours. Following probing with antibodies, chemiluminescent detection is completed with ECL western blotting reagents (Amersham, Buckinghamshire, UK). Quantification is performed by measuring band intensity using ImageJ software and calculating the ratio of protein of interesting to RAN the internal loading control.

### Results

### Effect of Nobiletin, 6-OH-Nobiletin, and 7-OH-Nobiletin on the Proliferation of Pre-Confluent 3T3-L1 Adipocytes

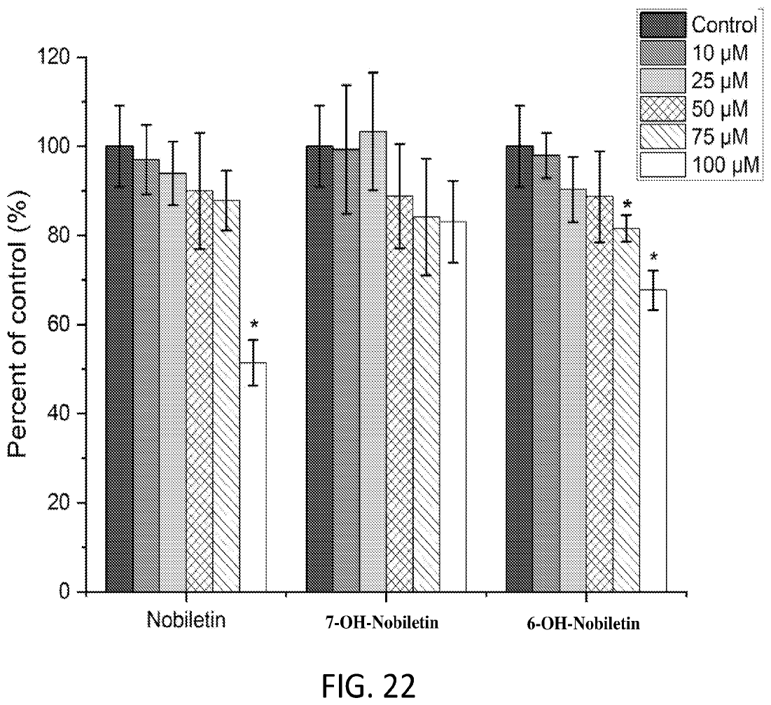

To access the safe concentration range of nobiletin, 6-OH-nobiletin, and 7-OH-nobiletin, the viability of pre-confluent 3T3-L1 preadipocytes were evaluated in the presence or absence of series doses of the three compounds. As shown in FIG. 22, nobiletin did not demonstrated any inhibition effect on the proliferation of preadipocytes up to 75 μM. When the concentration of nobiletin reached 100 μM, cell viability was reduced by 50%. 7-OH-nobiletin did not present significant effect on cell proliferation even at 100 μM. In contrast, 6-OH-nobiletin, at 75 μM and 100 μM, reduced cell viability to around 82% and 65%, respectively.

6-OH-Nobiletin is More Potent than Nobiletin and 7DMN in Reducing Lipid Accumulation in Differentiating 3T3-L1 Adipocytes

To compare the anti-adipogenic efficacy of nobiletin, 7-OH-nobiletin and 6-OH-nobiletin, 3T3-L1 preadipocytes were induced by culturing in DM to differentiate in the presence of various concentrations of the three compounds. Oil red O staining was carried out at day 8.

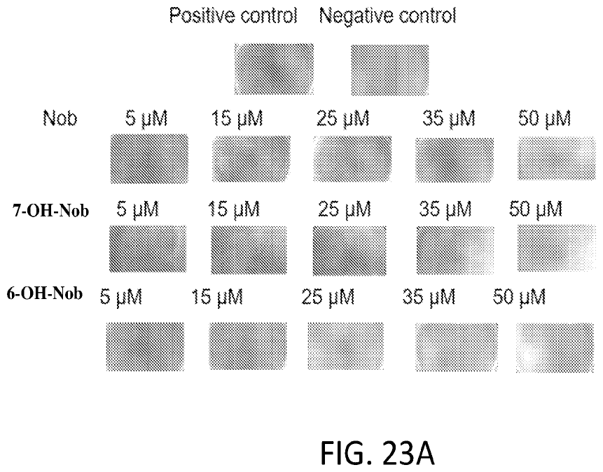

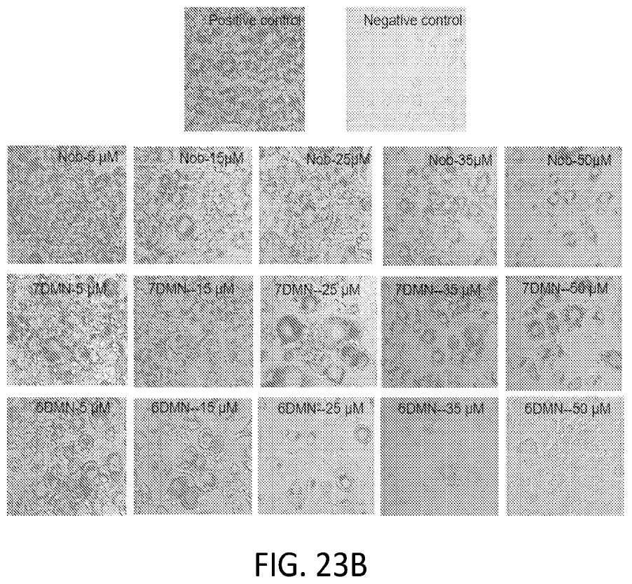

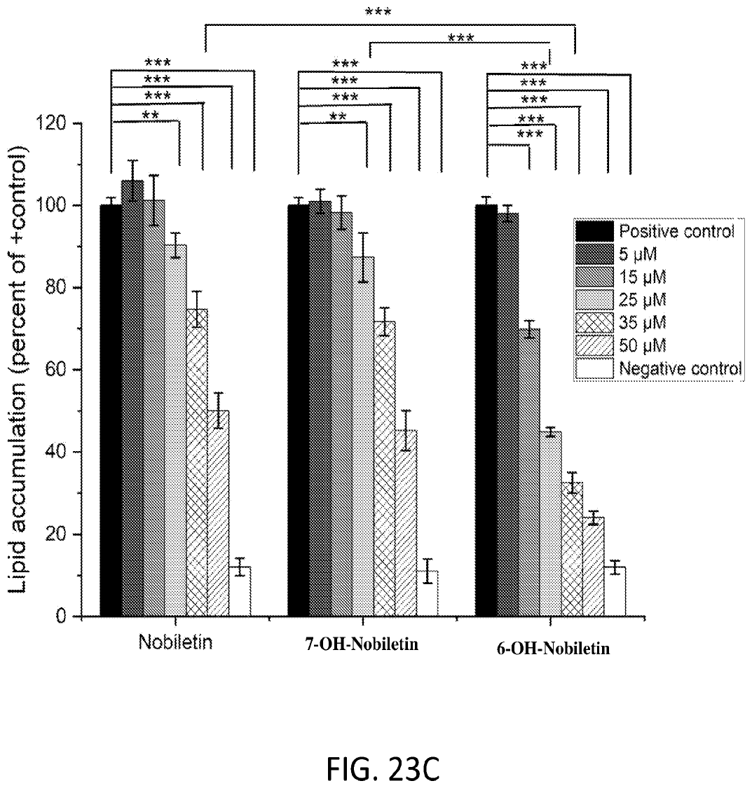

As shown in FIG. 23A, the positive control was dyed in red while the negative control did not show red color. Meanwhile, FIG. 23B demonstrated that millions of intracellular lipid droplets formed in the positive control while no droplets were seen in the negative control group. The phenomenon indicated that the preadipocytes were successfully differentiated into mature adipocytes characterized by intracellular macrovasicular steatosis. The lipid accumulation was quantified by Oil Red O staining.

As FIG. 23C shows, nobiletin, 7-OH-nobiletin and 6-OH-nobiletin had obvious suppressive effect on intracellular lipid accumulation and the effect was on a dose-dependent way. Interestingly, compared with nobiletin, 6-OH-nobiletin showed stronger potency in each concentration level. For example, 6-OH-nobiletin (15 M) reduced intracellular lipid content to 69.8±2.1%. At dose of 50 M, 6-OH-nobiletin reduced lipid formation to 24.3±4.2%, while nobiletin decreased the lipid accumulation to 45.2±4.9%.

### 6-OH-Nobiletin Suppressed the Adipogenesis of 3T3-L1 Cells in the Early and Late Differentiation Stages of Adipocyte Maturation

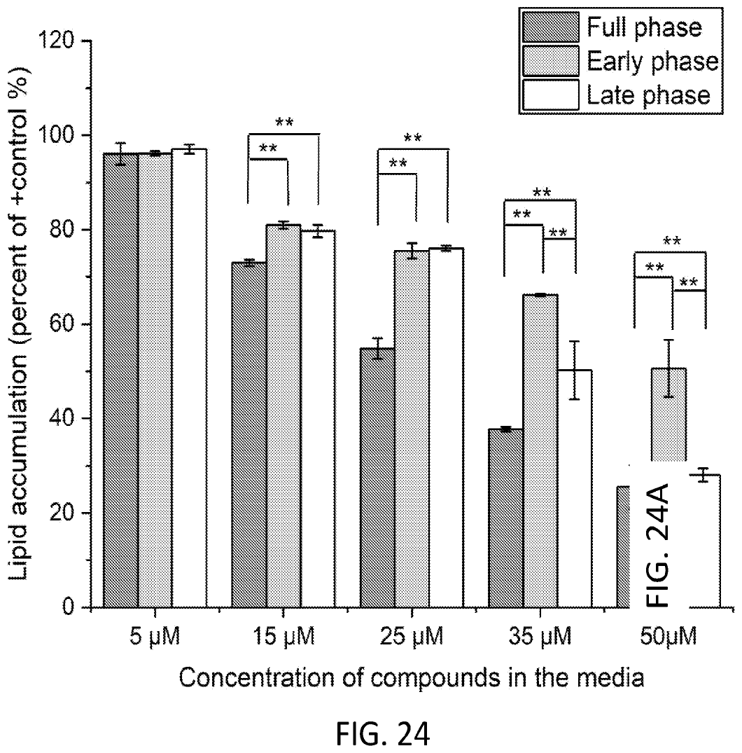

During adipocyte maturation, 3T3-L1 preadipocytes undergo multiple morphologic and genetic changes. The cell adipogenesis pathway is composed of two phases of differentiation, an early phase differentiation from day 0 to day 3 and a late phase differentiation from day 4 to day 8 (Tomiyama et al., 1999; Inokawa et al., 2016) To determine the effects of 6-OH-nobiletin on each phase, we treated the cells with 6-OH-nobiletin during early phase differentiation (day 0 to day 3), and late phase differentiation (day 4 to day 8). As shown in FIG. 24, both the early phase treatment and the late phase treatment suppressed the lipid accumulation in a dose-dependent matter. Interestingly, FIG. 24 shows that when the concentration of 6-OH-nobiletin was at 15 μM and 25 PM, the anti-adipogenesis capacity of early-phase and late-phase treatments were comparable; however, with further increase of the dose (35 μM and 50 μM), late phase treatment presented stronger suppressive activity than the early phase treatment. For example, at 50 μM, late-phase treatment inhibited lipid accumulation by 72% while early phase treatment decreased lipid content by 50%.

### Effects of 6-OH-Nobiletin on Post-Confluent Mitotic Clonal Expansion and Cell Cycle Progression of 3T3-L1 Cells During the Early Stage of Differentiation

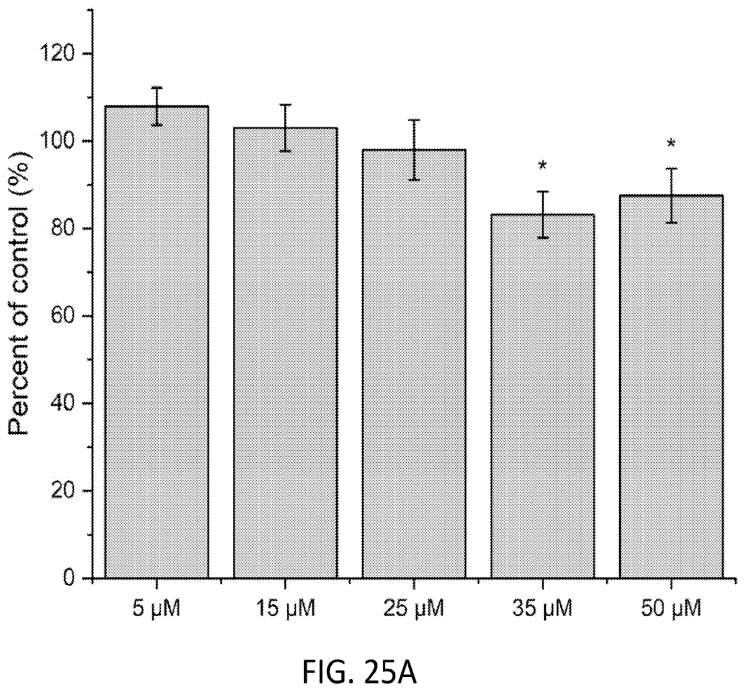

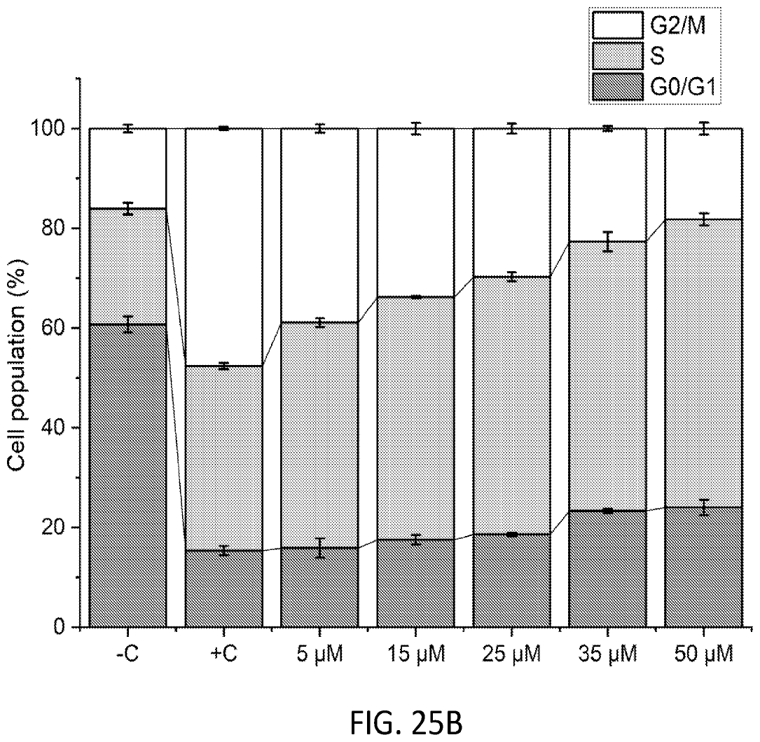

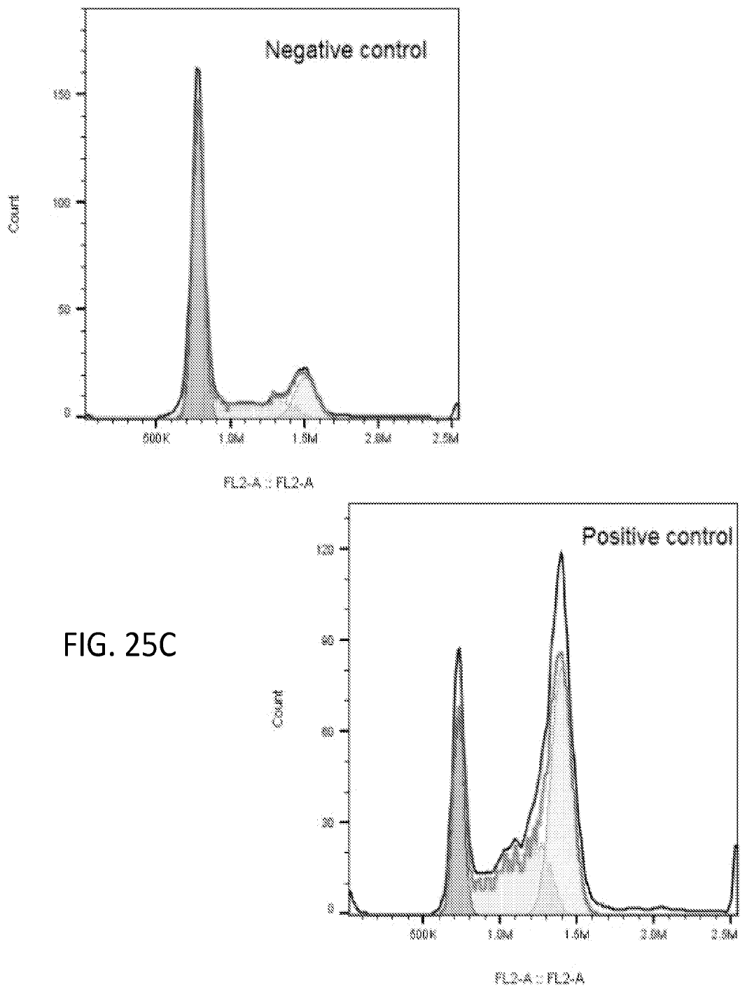

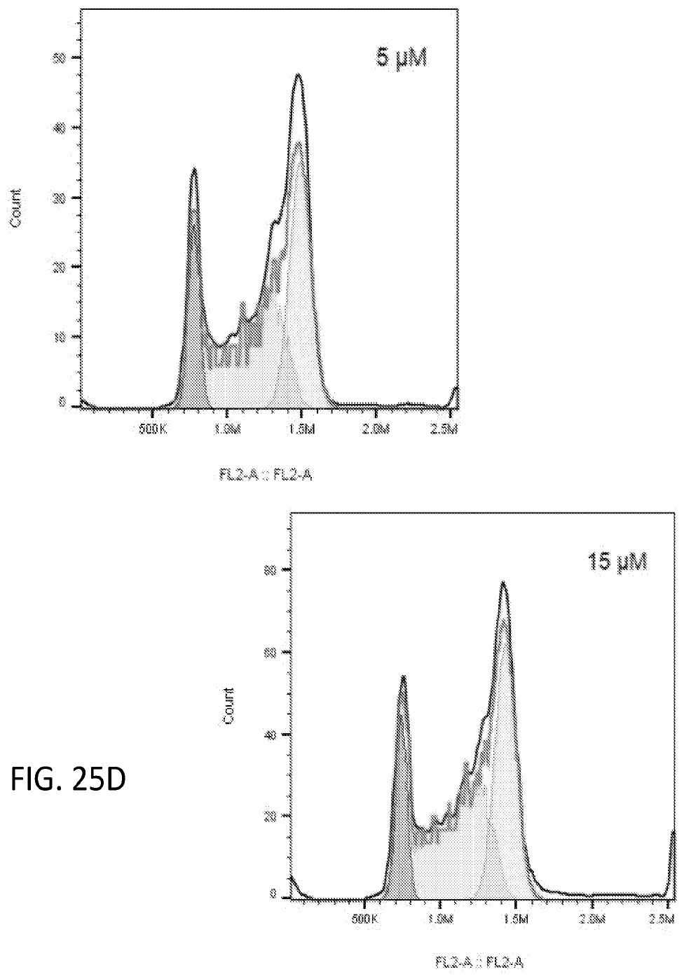

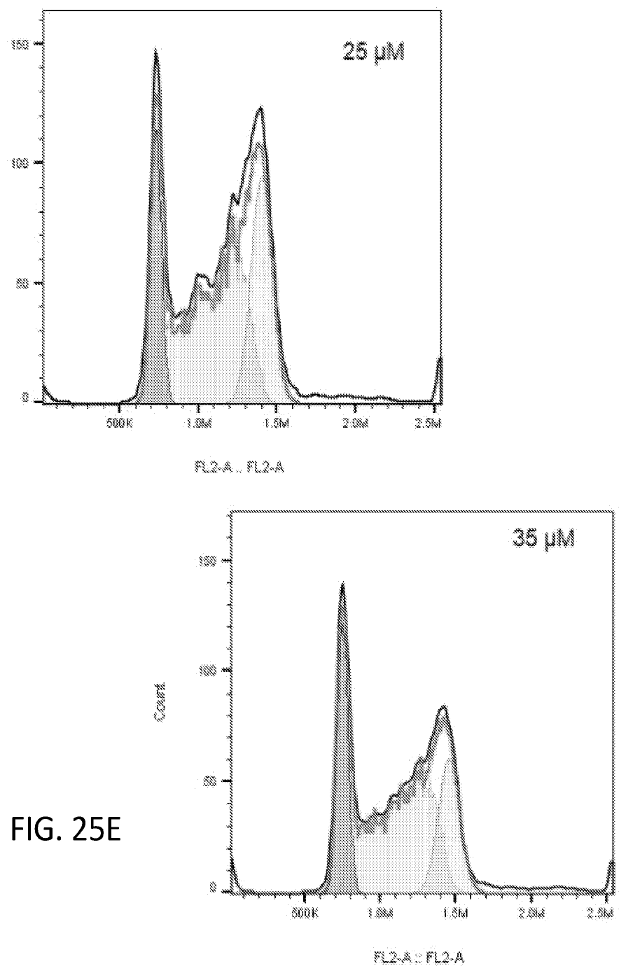

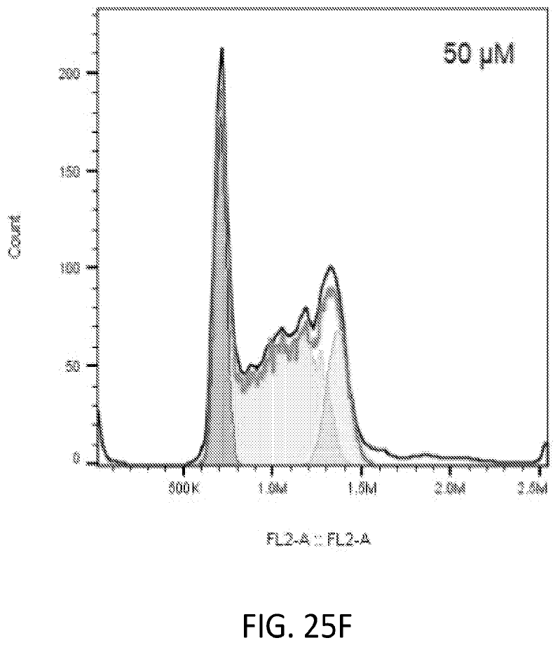

After being exposed to differentiation inducers, post-confluent 3T3-L1 cells would jump out of growth-arrest and undergo mitotic clonal expansion (MCE) during the first 72 hours before the terminal differentiation (Tang et al., 2003). MCE goes in parallel with early differentiation and therefore plays a critical role in the adipocyte differentiation process (Patel, & Lane, 2000). Since early phase treatment showed marked suppress on adipogenesis, it is expected that OH-PMF treatment would affect the preadipocyte proliferation step. MTT assay which reflects the total number of living cells were performed on post-confluent cells. As demonstrated in FIG. 25A, there was a slight decrease on the cell viability with 6-OH-nobiletin increasing from 5 μM to 25 μM. But there was no significant difference when taking standard error into consideration. At 35 μM and 50 μM, cell viability was significantly decreased to 83.1±6.2%, 85.6±5.3%, respectively.

Next, flow cytometry with PI staining was applied to analyze cell cycle profiles of treated and untreated cells. FIG. 25B proved that 6-OH-nobiletin significantly delayed the progression of the cell cycle and increased the S population in a dose-dependent manner. As shown in FIGS. 25B and 25C, the negative control group had approximately 61%, 22% and 17% of 3T3-L1 cells in the G0/G1, S and G2/M phase, respectively. The positive control group showed an approximate decrease in G0/G1 population of 18% and an increase in the G2/M population of 44%, which revealed that the inducers in the DM successfully initiated preadipocyte differentiation. However, the application of 6-OH-nobiletin inhibited the MCE process. As shown in FIGS. 25B and 25C, 6-OH-nobiletin significantly increased the S population and decreased the G2/M population, and this effect was in a dose-dependent manner. For example, at dose 5 μM, 40% of the preadipocytes were in G2/M phase and 42% of cells were in S phase. When the concentration of 6-OH-nobiletin increased to 50 μM, the population in G2/M phase decreased to 20% and S phase increased to 60%. In summary, 6-OH-nobiletin effectively blocked MCE in the S phase.

### Effects of 6-OH-Nobiletin on the Expression of Transcription Factors and Lipogenesis-Specific Genes

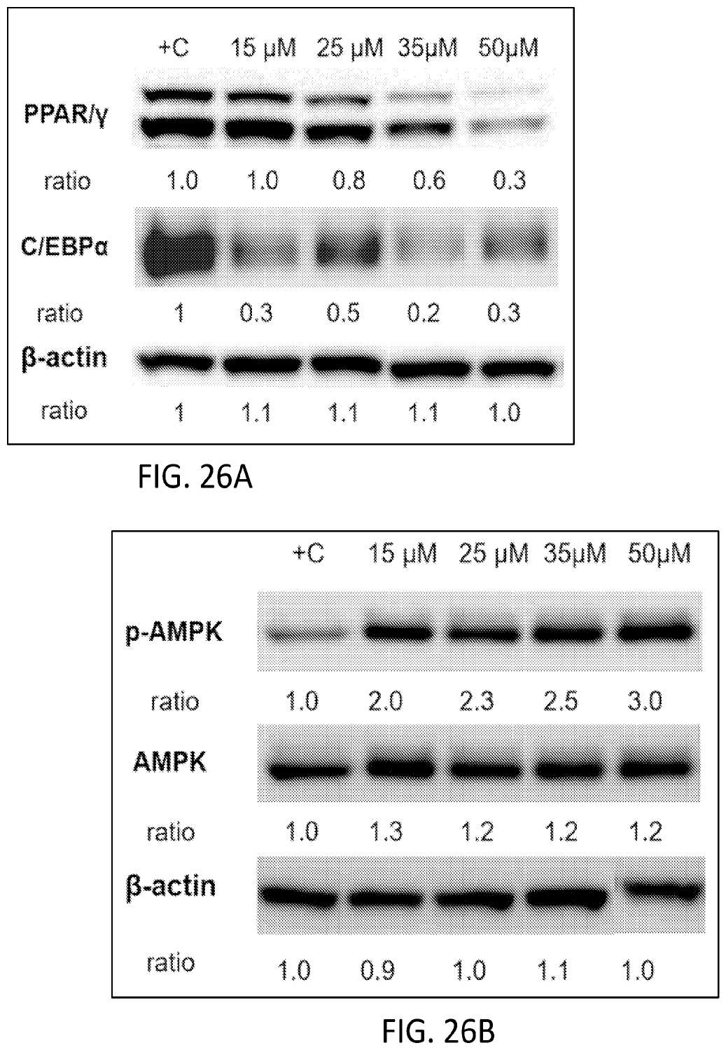

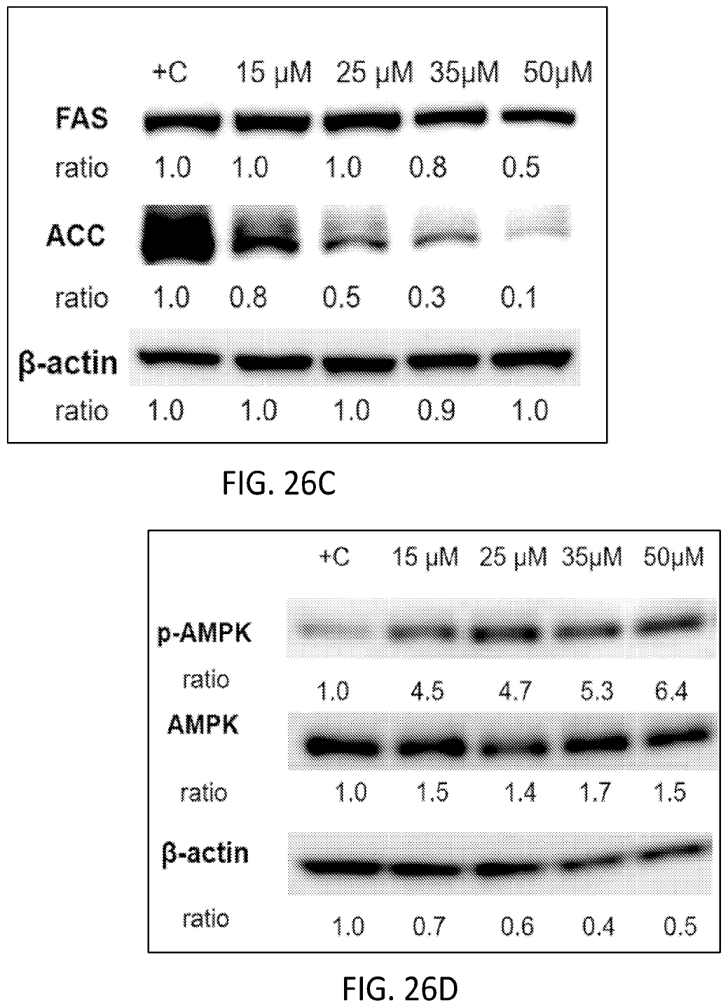

To investigate the molecular mechanism underlying the anti-adipogenic effect of 6-OH-nobiletin we analysed expression levels of key transcription regulators that are essential to adipocyte differentiation and adipogenic genes involved in glucose uptake and lipid metabolism. The key transcription factors were detected on day 3 after the stimulation of 3T3-L1 differentiation. As shown in FIG. 26A, 6-OH-nobiletin dose-dependently suppressed the expression level of PPAR/7. The production of C/EBPa was also greatly reduced.

With the induction of C/EBPa and PPAR/γ (multiple adipocyte-specific genes that control insulin action) lipid synthesis and transport are abundantly expressed in the terminal differentiation stage and directly establish the mature adipocyte phenotype (Farmer, 2006). The two critical adipogenic genes FAS and ACC were analyzed for the late phase treatment group. As demonstrated in FIG. 26B, 6-OH-nobiletin significantly suppressed their expression and the efficacy was positively related to the doses.

AMP-activated protein kinase (AMPK) is a critical energy sensor that is involved in cellular energy homeostasis (Daval, Foufelle, & Ferre, 2006). FIGS. 25C and 25D demonstrated that during the early and terminal differentiation processes, 6-OH-nobiletin greatly enhanced the expression and phosphorylation of AMPK.

### DISCUSSION

Nobiletin is highly recognized as a promising, food-derived agent to help prevent obesity. However, its visual bio-functionality is directly related to its metabolites which are produced or existed in organs or tissues. 6-OH-nobiletin and 7-OH-nobiletin are two main nobelitin metabolites formed in liver. This study was designed to provide an answer to a major gap in our understanding of anti-adipogenesis efficacy of 6-OH-nobiletin and 7-OH-nobiletin and would promote the development of nobiletin as oral or intravenous medication. The data indicates that 6-OH-nobiletin is much stronger than 7-OH-nobiletin and nobiletin in suppressing lipid accumulation in 3T3-L1 cells.

In addition, our results demonstrated that 6-OH-nobiletin did not significantly affect the pre-confluent cell proliferation. Therefore, its lipogenesis inhibition effect was not due to cytotoxicity. Instead, 6-OH-nobiletin interfered with both the early and late differentiation stages. The early differentiation goes in parallel with two rounds of mitotic clonal expansion (MCE) in the first 36 to 72 hours. Our results proved that 6-OH-nobiletin inhibited FM-induced clonal expansion of 3T3-L1 cells at 35 μM and 50 μM (FIG. 26A) and blocked cell cycle progression in the S phase. During the MCE process, various adipogenetic transcription factors and regulators are produced. To understand the molecular mechanism underlying the MCE blockage effect of 6-OH-nobiletin in the early differentiation processes, the expression level of two key transcription factors PPAR/γ and C/EBP R were studied. Our results demonstrated that 6-OH-nobiletin significantly suppressed the expression of the two regulators.

PPAR/γ is essential for the differentiation process of pre-adipocytes to adipocytes. C/EBPα is another key regulator that is expressed at the adipogenic initiation stage and works synergistically with PPAR/γ to induce its own expression and induce the activation of a number of downstream target genes (Moseti, Regassa, & Kim, 2016). Therefore, the data indicates that the lipid formation inhibition effect of 6-OH-nobiletin is related to the decreased expression of the early transcription factors.

FAS and ACC are two critical lipogenesis specific enzymes involved in the fatty acid biosynthesis. The suppressive effect of 6-OH-nobiletin on FAS and ACC explains the prominent anti-adipogenesis effect shown in the late-phase treatment group. These results indicate that hydroxylated polymethoxyflavones could effectively inhibit the expression of transcription factors PPAR/γ as well as adipogetic genes FAS and ACC.

The data also indicates that the AMP-activated protein kinase (AMPK) pathway was highly activated by the treatment of 6-OH-nobiletin. The activation of AMPK dose boosts energy expenditure and inhibits the expression of adipogenetic genes like PPAR/γ (Lee et al., 2009), FAS and ACC (Motoshima et al., 2006). Therefore, the data indicates that 6-OH-nobiletin suppresses adipogenesis and lipogenesis by activating AMPK which in turn influence PPAR/γ, C/EBPα, FAS and ACC.

In the past few years, the activation of brown adipocytes and trans-differentiation of white adipose tissue to brown adipocyte has gained great popularity. Browning of white adipocytes (beiging) has been proposed to be a therapeutic strategy against obesity. Lone et al., (2018) reported that nobiletin induces brown adipocyte-like phenotype and ameliorates stress in 3T3-L1 adipocytes. Meanwhile, several other bioactive compounds or fruit extracts such as curcumin (Lone et al., 2016), quercetin (Aziz et al., 2017) and strawberry methanolic extract (Forbes-Hernindez et al., 2020) have been reported to suppressing obesity by promoting thermogenesis and white adipocyte tissue browning. In view of these articles, the data presented herein indicates that OH-PMFs have an effect on brown adipocyte-like phenotype.

Mitochondrion is one of the essential organelles in a cell. It plays a pivotal role in cellular processes such as energy supply (ATP), free radical generation, metabolites production for biosynthesis (Starko, 2008). During the ATP production process (oxidative phosphorylation), reactive oxygen species (ROS) or reactive nitrogen species (RNS) are inevitably produced (Kalyanaraman et al., 2019). In homeostatic imbalance conditions, excessive ROS and RNS would impair DNA, proteins, lipids, leading to micothondria dysfunction (Tan et al., 2021, Kirtonia et al., 2020). Of note, it is well recognized that obesity is closely linked to mitochondrial dysfunction (Yin et al., 2014; de et al., 2018). For instance, mitochondria would undergo marked changes in mitochondrial mass, oxygen consumption, oxidative stress, glycolysis capacity during adipogenesis and throughout the progression of metabolic disease (Sarparanta et al., 2017; Basse er al., 2017). Therefore, the modulation of mitochondrial oxygen consumption and oxidative stress have therapeutic potential for the treatment of obesity (Heidari et al., 2018; Pan et al., 2018).

In the differentiation process of 3T3-L1 cells. Mitochondria will undergo dynamic regulations of network and oxidative function. Since 6-OH-nobiletin, 7-OH-nobiletin, and nobiletin showed prominent anti-adipogenesis activity, the data indicates that they have bio-functionality from the aspect of mitochondria. The data further indicates that OH-PMFs have an effect on oxidative stress, glycolytic capacity, mitophagy, mitochondrial oxygen consumption, fussion and fission and so on.

Overall, our study has great importance in uncovering the anti-obesity mechanism of 6-OH-nobiletin as well as opens a new window for investigating the mode of action of nobelitin in preventing obesity.

The present study demonstrated that the 6-hydroxylated nobiletin (6DMN), also known as 6-OH-nobiletin, was more potent than nobiletin in inhibiting adipocyte differentiation and it caused cell delay. 6-OH-nobiletin did not only interfere the early differentiation process but also block the terminal differentiation pathway. Molecular analysis revealed that 6-OH-nobiletin significantly decreased the expression level of transcription factors as well as lipogenesis specific genes involved in the adipocyte differentiation. Meanwhile, the energy sensor AMPK was highly activated. These findings prove that 6-OH-nobiletin is an effective therapeutic for the management of obesity.

### REFERENCES

Blüher, M. (2019). Obesity: global epidemiology and pathogenesis. Nature Reviews Endocrinology, 15(5), 288-298.
Aziz, S. A., Wakeling, L. A., Miwa, S., Alberdi, G., Hesketh, J. E. and Ford, D., 2017. Metabolic programming of a beige adipocyte phenotype by genistein. Molecular Nutrition & Food Research, 61(2), p.1600574.
Basse, A. L., Isidor, M. S., Winther, S., Skjoldborg, N. B., Murholm, M., Andersen, E. S., Pedersen, S. B., Wolfrum, C., Quistorff, B. and Hansen, J. B., 2017. Regulation of glycolysis in brown adipocytes by HIF-1α. Scientific reports, 7(1), pp.1-15.
Choi, Y., Kim, Y., Ham, H., Park, Y., Jeong, H. S. and Lee, J. 2011. Nobiletin suppresses adipogenesis by regulating the expression of adipogenic transcription factors and the activation of AMP-activated protein kinase (AMPK). J. Agric. Food Chem. 59, 12843-12849.
Chooi, Y. C., Ding, C., & Magkos, F. (2019). The epidemiology of obesity. Metabolism, 92, 6-10.
Daval, M., Foufelle, F., & Ferré, P. (2006). Functions of AMP-activated protein kinase in adipose tissue. The Journal of physiology, 574(1), 55-62. de Mello, A. H., Costa, A. B., Engel, J. D. G. and Rezin, G. T., 2018. Mitochondrial dysfunction in obesity. Life sciences, 192, pp.26-32.
Farmer, S. R., 2006. Transcriptional control of adipocyte formation. Cell metabolism, 4(4), pp. 263-273.
Fève, B. (2005). Adipogenesis: cellular and molecular aspects. Best Practice & Research Clinical Endocrinology & Metabolism, 19(4), 483-499.
Forbes-Hernindez, T. Y., Cianciosi, D., Ansary, J., Mezzetti, B., Bompadre, S., Quiles, J. L., Giampieri, F. and Battino, M., 2020. Strawberry (Fragaria× ananassa cv. Romina) methanolic extract promotes browning in 3T3-L1 cells. Food & Function, 11(1), pp.297-304.
Guo, J., Cao, Y., Ho, C. T., Jin, S., & Huang, Q. (2017). Aged citrus peel (chenpi) extract reduces lipogenesis in differentiating 3T3-L1 adipocytes. Journal of Functional Foods, 34, 297-303.
Heidari, R., Ghanbarinejad, V., Mohammadi, H., Ahmadi, A., Ommati, M. M., Abdoli, N., Aghaei, F., Esfandiari, A., Azarpira, N. and Niknahad, H., 2018. Mitochondria protection as a mechanism underlying the hepatoprotective effects of glycine in cholestatic mice. Biomedicine & Pharmacotherapy, 97, pp.1086-1095.
Inokawa, A., Inuzuka, T., Takahara, T., Shibata, H., & Maki, M. (2016). Tubby-like protein superfamily member PLSCR3 functions as a negative regulator of adipogenesis in mouse 3T3-L1 preadipocytes by suppressing induction of late differentiation stage transcription factors. Bioscience reports, 36(1), e00287.
Kalyanaraman, B., Cheng, G., Zielonka, J. and Bennett, B., 2019. Low-temperature EPR spectroscopy as a probe-free technique for monitoring oxidants formed in tumor cells and tissues: implications in drug resistance and OXPHOS-targeted therapies. Cell biochemistry and biophysics, 77(1), pp.89-98.
Kim, Y. J., Choi, M. S., Woo, J. T., Jeong, M. J., Kim, S. R. and Jung, U. J. 2017. Long-term dietary supplementation with low-dose nobiletin ameliorates hepatic steatosis, insulin resistance, and inflammation without altering fat mass in diet-induced obesity. Molecular nutrition & food research, 61,1600889.
Kirtonia, A., Sethi, G. and Garg, M., 2020. The multifaceted role of reactive oxygen species in tumorigenesis. Cellular and Molecular Life Sciences, 77(22), pp.4459-4483.
Koga, N., Matsuo, M., Ohta, C., Haraguchi, K., Matsuoka, M., Kato, Y., Ishii, T., Yano, M., Ohta, H. 2007. Comparative study on nobiletin metabolism with liver microsomes from rats, guinea pigs and hamsters and rat cytochrome P450. Biol. Pharm. Bull. 30, 2317-2323.
Koga, N., Ohta, C., Kato, Y., Haraguchi, K., Endo, T., Ogawa, K., . . . Yano, M. 2011. In vitro metabolism of nobiletin, a polymethoxy-flavonoid, by human liver microsomes and cytochrome P450. Xenobiotica. 41, 927-933.
Lee, Y. K., Lee, W. S., Hwang, J. T., Kwon, D. Y., Surh, Y. J., & Park, O. J. (2009). Curcumin exerts antidifferentiation effect through AMPKα-PPAR-γ in 3T3-L1 adipocytes and antiproliferatory effect through AMPKα-COX-2 in cancer cells. Journal of agricultural and food chemistry, 57(1), 305-310.
Lone, J., Choi, J. H., Kim, S. W. and Yun, J. W., 2016. Curcumin induces brown fat-like phenotype in 3T3-L1 and primary white adipocytes. The Journal of Nutritional Biochemistry, 27, pp.193-202.
Lone, J., Parray, H. A. and Yun, J. W., 2018. Nobiletin induces brown adipocyte-like phenotype and ameliorates stress in 3T3-L1 adipocytes. Biochimie, 146, pp.97-104.
Moseti, D., Regassa, A., & Kim, W. K. (2016). Molecular regulation of adipogenesis and potential anti-adipogenic bioactive molecules. International journal of molecular sciences, 17(1), 124.
Motoshima, H., Goldstein, B. J., Igata, M., & Araki, E. (2006). AMPK and cell proliferation-AMPK as a therapeutic target for atherosclerosis and cancer. The Journal of physiology, 574(1), 63-71.
Oshitari, T., Okuyama, Y., Miyata, Y., Kosano, H., Takahashi, H., & Natsugari, H. (2011). Nobiletin metabolites: Synthesis and inhibitory activity against matrix metalloproteinase-9 production. Bioorganic & medicinal chemistry letters, 21(15), 4540-4544.
Pan, Y., Zhao, W., Zhao, D., Wang, C., Yu, N., An, T., Mo, F., Liu, J., Miao, J., Lv, B. and Gu, Y., 2018. Salvianolic acid B improves mitochondrial function in 3T3-L1 adipocytes through a pathway involving PPARγ coactivator-1α (PGC-1α). Frontiers in Pharmacology, 9, p.671.
Patel, Y. M., & Lane, M. D. (2000). Mitotic clonal expansion during preadipocyte differentiation: calpain-mediated turnover of p27. Journal of Biological Chemistry, 275(23), 17653-17660.
Sarparanta, J., Garcia-Macia, M. and Singh, R., 2017. Autophagy and mitochondria in obesity and type 2 diabetes. Current diabetes reviews, 13(4), pp.352-369.
Starkov, A. A., 2008. The role of mitochondria in reactive oxygen species metabolism and signaling. Annals of the New York Academy of Sciences, 1147(1), pp.37-52.
Su, J. D., Yen, J. H., Li, S., Weng, C. Y., Lin, M. H., Ho, C. T. and Wu, M. J., 2012. 3′, 4′-Didemethylnobiletin induces phase II detoxification gene expression and modulates PI3K/Akt signaling in PC12 cells. Free Radical Biology and Medicine, 52(1), pp.126-141.
Surichan, S., Arroo, R. R., Ruparelia, K., Tsatsakis, A. M. and Androutsopoulos, V. P., 2018. Nobiletin bioactivation in MDA-MB-468 breast cancer cells by cytochrome P450 CYP1 enzymes. Food and chemical toxicology, 113, pp.228-235.
Tan, Y. Q., Zhang, X., Zhang, S., Zhu, T., Garg, M., Lobie, P. E. and Pandey, V., 2021. Mitochondria: The metabolic switch of cellular oncogenic transformation. Biochimica et Biophysica Acta (BBA)-Reviews on Cancer, 1876(1), p.188534.
Tang, Q. Q., Otto, T. C., & Lane, M. D. (2003). Mitotic clonal expansion: a synchronous process required for adipogenesis. Proceedings of the National Academy of Sciences, 100(1), 44-49.
Tomiyama, K., Nishio, E., & Watanabe, Y. (1999). Both wortmannin and simvastatin inhibit the adipogenesis in 3T3-L1 cells during the late phase of differentiation. The Japanese Journal of Pharmacology, 80(4), 375-378.
Wu, X., Song, M., Wang, M., Zheng, J., Gao, Z., Xu, F., Zhang, G. and Xiao, H. 2015. Chemopreventive effects of nobiletin and its colonic metabolites on colon carcinogenesis. Mol. Nutr. Food Res. 59, 2383-2394.
Yin, X., Lanza, I. R., Swain, J. M., Sarr, M. G., Nair, K. S. and Jensen, M. D., 2014. Adipocyte mitochondrial function is reduced in human obesity independent of fat cell size. The Journal of Clinical Endocrinology & Metabolism, 99(2), pp. E209-E216.
Zhang, M., Xin, Y., Feng, K., Yin, B., Kan, Q., Xiao, J., Cao, Y., Ho, C. T. and Huang, Q. 2020a. Comparative analyses of bioavailability, biotransformation, and excretion of nobiletin in lean and obese rats. J. Agric. Food Chem. 68, 10709-10718.
Linhart, H. G., Ishimura-Oka, K., DeMayo, F., Kibe, T., Repka, D., Poindexter, B., . . . & Darlington, G. J. (2001). C/EBPα is required for differentiation of white, but not brown, adipose tissue. Proceedings of the National Academy of Sciences, 98(22), 12532-12537.
Lai, C. S., Ho, M. H., Tsai, M. L., Li, S., Badmaev, V., Ho, C. T., & Pan, M. H. (2013). Suppression of adipogenesis and obesity in high-fat induced mouse model by hydroxylated polymethoxyflavones. Journal of agricultural and food chemistry, 61(43), 10320-10328.
Wolfrum, C., Quistorff, B. and Hansen, J. B., 2017. Regulation of glycolysis in brown adipocytes by HIF-1α. Scientific reports, 7(1), pp.1-15.
Styskal, J., Van Remmen, H., Richardson, A., & Salmon, A. B. (2012). Oxidative stress and diabetes: what can we learn about insulin resistance from antioxidant mutant mouse models?. Free Radical Biology and Medicine, 52(1), 46-58.
Pan, M. H., Li, M. Y., Tsai, M. L., Pan, C. Y., Badmaev, V., Ho, C. T., & Lai, C. S. (2019). A mixture of citrus polymethoxyflavones, green tea polyphenols and lychee extracts attenuates adipogenesis in 3T3-L1 adipocytes and obesity-induced adipose inflammation in mice. Food & function, 10(12), 7667-7677.
Hadrich, F., & Sayadi, S. (2018). Apigetrin inhibits adipogenesis in 3T3-L1 cells by downregulating PPARγ and CEBP-α. Lipids in health and disease, 17(1), 1-8.
Hardie, D. G. (2008). AMPK: a key regulator of energy balance in the single cell and the whole organism. International journal of obesity, 32(4), S7-S12.Farmer, S. R. Transcriptional control of adipocyte formation. Cell Metab. 2006, 4, 263-273.
Gregoire, F. M., Smas, C. M., & Sul, H. S. (1998). Understanding adipocyte differentiation. Physiological reviews, 78(3), 783-809.
Eguchi, A., Murakami, A., Li, S., Ho, C. T., & Ohigashi, H. (2007). Suppressive effects of demethylated metabolites of nobiletin on phorbol ester-induced expression of scavenger receptor genes in THP-1 human monocytic cells. Biofactors, 31(2), 107-116.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

1. A method of producing hydroxylated polymethoxyflavone metabolites (OH-PMFs), the method comprising, culturing Filobasidium magnum in the presence of a polymethoxyflavone (PMF) under conditions which permit the hydroxylation of said PMF metabolites.

2. The method of claim 1, wherein the Filobasidium magnum is NRRL Deposit No. Y-68198.

3. The method of claim 1, wherein the metabolite is an OH-PMF listed in Table 8.

4. The method of claim 1, wherein said OH-PMF is a least one of 6-OH-PMF or 7-OH-PMF.

5. The method of claim 1, further comprising and extracting the OH-PMF metabolite.

6. The method of claim 1, wherein the PMF is at least one of nobiletin, sinensetin, tangeretin and heptamethoxyflavone.

7. The method of claim 1, wherein said PMF is nobiletin and metabolite is 6-OH-nobiletin or 7-OH-nobiletin.

8. The method of claim 1, wherein
a)the conditions comprise a pH of about 4.5, about 6.0, or about 7.5;
b) the media is incubated at about 20° C., at about 26° C. or at about 37° C.;
c) the media is incubated for between about 2 to about 5 days after contact with the PMF; and, or
d) the media comprises Chenpi powder 20 g/L, glucose 10 g/L, sucrose 10 g/L, beef extract 0.2 g/L, peptone 10 g/L, yeast extract 10 g/L, MgSO₄ 0.05 g/L, KH2PO4 2 g/L, NaCl 0.5 g/L.

9.-11. (canceled)

12. The method of claim 1, wherein the PMF is dissolved in DMSO prior to contact with the yeast.

13. The method of claim 1, wherein the PMF is in an aqueous solution with a stabilizer.

14. The method of claim 13, wherein the PMF is in a nanodispersion.

15. The method of claim 14, wherein the nanodispersion is prepared using a media-milling technique.

16. The method of claim 13, wherein the nanodispersion was milled for about 3 hours.

17. The method of claim 14, wherein the nanodispersion is milled to around 800 nm.

18. The method of claim 14, wherein the stabilizer is hydrophobically modified starch.

19. A method for modulating adipolipogenesis, comprising administering to a mammal in need thereof, an effective amount of 6-OH-nobiletin or 7-OH-nobiletin or a combination thereof.

20. The method of claim 19, wherein a combination of 6-OH-nobiletin and 7-OH-nobiletin is administered in a ratio of between 1:40 and 1:50.

21. (canceled)

22. The method of claim 19, wherein the 6-OH-nobiletin or 7-OH-nobiletin or a combination thereof is present in a pharmaceutically acceptable carrier.

23. The method of claim 19, wherein obesity symptoms are assessed following treatment.
